SEQUENCE LISTING (148 sequences)

SEQ ID NO: 1 - DNA, 23 bp, Artificial Sequence
  Synthetic
cctggcacagagaagctgaatga

SEQ ID NO: 2 - DNA, 25 bp, Artificial Sequence
  Synthetic
cagggcgtgagatgataataagcaa

SEQ ID NO: 3 - DNA, 25 bp, Artificial Sequence
  Synthetic
cctggcacagagaagctgaatgaaa

SEQ ID NO: 4 - DNA, 22 bp, Artificial Sequence
  Synthetic
ctcgcagggcgtgagatgataa

SEQ ID NO: 5 - DNA, 24 bp, Artificial Sequence
  Synthetic
tgtgcgtccagccacatcacattg

SEQ ID NO: 6 - DNA, 24 bp, Artificial Sequence
  Synthetic
tactggggtcttgggctcgtgctg

SEQ ID NO: 7 - DNA, 27 bp, Artificial Sequence
  Synthetic
cagccacatcacattggagtcacaatc

SEQ ID NO: 8 - DNA, 24 bp, Artificial Sequence
  Synthetic
agctgctactggggtcttgggctc

SEQ ID NO: 9 - DNA, 18 bp, Artificial Sequence
  Synthetic
gagccaccacgcgcccat

SEQ ID NO: 10 - DNA, 25 bp, Artificial Sequence
  Synthetic
cctgaagagcaggggactaactcca

SEQ ID NO: 11 - DNA, 19 bp, Artificial Sequence
  Synthetic
catgagccccgggagcagc

SEQ ID NO: 12 - DNA, 25 bp, Artificial Sequence
  Synthetic
cctgaagagcaggggactaactcca

SEQ ID NO: 13 - DNA, 18 bp, Artificial Sequence
  Synthetic
ccaacaaaggagccacca

SEQ ID NO: 14 - DNA, 27 bp, Artificial Sequence
  Synthetic
ggactaactccagttactacatcctga

SEQ ID NO: 15 - DNA, 23 bp, Artificial Sequence
  Synthetic
cccggtacatctgaaagccggga

SEQ ID NO: 16 - DNA, 20 bp, Artificial Sequence
  Synthetic
actggcggatttccacgggc

SEQ ID NO: 17 - DNA, 20 bp, Artificial Sequence
  Synthetic
tctgtcgctctgtcgcctgc

SEQ ID NO: 18 - DNA, 22 bp, Artificial Sequence
  Synthetic
tggtgatacctgtctgagcggg

SEQ ID NO: 19 - DNA, 45 bp, Artificial Sequence
  Synthetic
ggataatacgactcactatagggcctcatcctgaccagcacttca

SEQ ID NO: 20 - DNA, 20 bp, Artificial Sequence
  Synthetic
ttcgggaggaggcaaacgag

SEQ ID NO: 21 - DNA, 42 bp, Artificial Sequence
  Synthetic
ggataatacgactcactatagggcctcgctcccttcatctgg

SEQ ID NO: 22 - DNA, 20 bp, Artificial Sequence
  Synthetic
tcgtccaccgtgggctggta

SEQ ID NO: 23 - DNA, 47 bp, Artificial Sequence
  Synthetic
ggataatacgactcactatagggacaacgagatcgtgatgatgaatc

SEQ ID NO: 24 - DNA, 21 bp, Artificial Sequence
  Synthetic
cagaacgctgtcgggttcgta

SEQ ID NO: 25 - DNA, 44 bp, Artificial Sequence
  Synthetic
ggataatacgactcactatagggtacgaacccgacagcgttctg

SEQ ID NO: 26 - DNA, 25 bp, Artificial Sequence
  Synthetic
tgcaattcatagaagtagaccgtgg

SEQ ID NO: 27 - DNA, 46 bp, Artificial Sequence
  Synthetic
ggataatacgactcactatagggcctatgaacgctctgagagcttg

SEQ ID NO: 28 - DNA, 23 bp, Artificial Sequence
  Synthetic
tccagcaggtggtagaactcctc

SEQ ID NO: 29 - DNA, 45 bp, Artificial Sequence
  Synthetic
ggataatacgactcactatagggtcaaccccgaggagttctacca

SEQ ID NO: 30 - DNA, 21 bp, Artificial Sequence
  Synthetic
atgcaccacatctggaaaggg

SEQ ID NO: 31 - DNA, 42 bp, Artificial Sequence
  Synthetic
ggataatacgactcactatagggtgcatctggaggaacagga

SEQ ID NO: 32 - DNA, 25 bp, Artificial Sequence
  Synthetic
cgttgcttatgagcttgatggtatc

SEQ ID NO: 33 - DNA, 47 bp, Artificial Sequence
  Synthetic
ggataatacgactcactatagggataccatcaagctcataagcaacg

SEQ ID NO: 34 - DNA, 21 bp, Artificial Sequence
  Synthetic
ttgcggaggatcaagttctgc

SEQ ID NO: 35 - DNA, 48 bp, Artificial Sequence
  Synthetic
ggataatacgactcactatagggtaactggagttagtcccctgctctt

SEQ ID NO: 36 - DNA, 26 bp, Artificial Sequence
  Synthetic
ccaggtttcctctccataacttacaa

SEQ ID NO: 37 - DNA, 46 bp, Artificial Sequence
  Synthetic
ggataatacgactcactatagggtgctccctttgtccagcagtgta

SEQ ID NO: 38 - DNA, 24 bp, Artificial Sequence
  Synthetic
ccagcagtaagagaaggtgcagac

SEQ ID NO: 39 - DNA, 44 bp, Artificial Sequence
  Synthetic
ggataatacgactcactatagggttgagccttccaagaagaggc

SEQ ID NO: 40 - DNA, 20 bp, Artificial Sequence
  Synthetic
tgtggccatggagtggtgag

SEQ ID NO: 41 - DNA, 45 bp, Artificial Sequence
  Synthetic
ggataatacgactcactatagggtccaaatgcacctgctcacttt

SEQ ID NO: 42 - DNA, 26 bp, Artificial Sequence
  Synthetic
cagtggagctaggagtgttagttcca

SEQ ID NO: 43 - DNA, 43 bp, Artificial Sequence
  Synthetic
ggataatacgactcactatagggaaaagctgcatcagttgcct

SEQ ID NO: 44 - DNA, 22 bp, Artificial Sequence
  Synthetic
ggactgccgtccttcctcatct

SEQ ID NO: 45 - DNA, 44 bp, Artificial Sequence
  Synthetic
ggataatacgactcactatagggacgatccccagtatcctttga

SEQ ID NO: 46 - DNA, 23 bp, Artificial Sequence
  Synthetic
cgctgtctggagtgttggaggaa

SEQ ID NO: 47 - DNA, 47 bp, Artificial Sequence
  Synthetic
ggataatacgactcactatagggcgactagcagagtttatttcctcc

SEQ ID NO: 48 - DNA, 23 bp, Artificial Sequence
  Synthetic
ctccgagatttatccaggcagtc

SEQ ID NO: 49 - DNA, 46 bp, Artificial Sequence
  Synthetic
ggataatacgactcactatagggctcagaagtggcttttgtgatgc

SEQ ID NO: 50 - DNA, 25 bp, Artificial Sequence
  Synthetic
aaggtggtagaactcttcagggtca

SEQ ID NO: 51 - DNA, 42 bp, Artificial Sequence
  Synthetic
ggataatacgactcactatagggaatgcctggagtttgaccc

SEQ ID NO: 52 - DNA, 21 bp, Artificial Sequence
  Synthetic
agctggctaacgatgtagcgg

SEQ ID NO: 53 - DNA, 48 bp, Artificial Sequence
  Synthetic
ggataatacgactcactatagggacagtcctgacactccagagacaga

SEQ ID NO: 54 - DNA, 23 bp, Artificial Sequence
  Synthetic
caccagaaatacagccccatagg

SEQ ID NO: 55 - DNA, 28 bp, Artificial Sequence
  Synthetic
caaaccatggggagcggctctacaagtc

SEQ ID NO: 56 - DNA, 46 bp, Artificial Sequence
  Synthetic
tggcttactttgtccacactccgggtgtatagcagcatggagcagc

SEQ ID NO: 57 - DNA, 45 bp, Artificial Sequence
  Synthetic
ctgctccatgctgctatacacccggagtgtggacaaagtaagcca

SEQ ID NO: 58 - DNA, 29 bp, Artificial Sequence
  Synthetic
caaaccatggaccgtttgggtccctttag

SEQ ID NO: 59 - DNA, 48 bp, Artificial Sequence
  Synthetic
gaagctgaatgaaaaagaaaaggaggcctatgaacgctctgagagctt

SEQ ID NO: 60 - DNA, 48 bp, Artificial Sequence
  Synthetic
aagctctcagagcgttcataggcctccttttctttttcattcagcttc

SEQ ID NO: 61 - DNA, 29 bp, Artificial Sequence
  Synthetic
gaaaccatgtcaggggatgtggcagtaga

SEQ ID NO: 62 - DNA, 22 bp, Artificial Sequence
  Synthetic
gaacagcacggacgcactttat

SEQ ID NO: 63 - DNA, 23 bp, Artificial Sequence
  Synthetic
gaattttcacgcagcacggacgc

SEQ ID NO: 64 - DNA, 27 bp, Artificial Sequence
  Synthetic
gaacacggacgcactttatttatatgt

SEQ ID NO: 65 - DNA, 30 bp, Artificial Sequence
  Synthetic
gaacggaccgttcacgcagcacggacgcac

SEQ ID NO: 66 - DNA, 32 bp, Artificial Sequence
  Synthetic
caacggaccgcagcacggacgcactttattta

SEQ ID NO: 67 - DNA, 33 bp, Artificial Sequence
  Synthetic
caacggaccgcacgcagcacggacgcactttat

SEQ ID NO: 68 - DNA, 25 bp, Artificial Sequence
  Synthetic
gaaaccatgcggcctcgctcccgga

SEQ ID NO: 69 - DNA, 27 bp, Artificial Sequence
  Synthetic
gaagtctgagtgcaagaaatggcaaac

SEQ ID NO: 70 - DNA, 24 bp, Artificial Sequence
  Synthetic
gaacaagaaatggcaaacaactgc

SEQ ID NO: 71 - DNA, 24 bp, Artificial Sequence
  Synthetic
caaaccatggccgcgcaggtcgcc

SEQ ID NO: 72 - DNA, 42 bp, Artificial Sequence
  Synthetic
gctcgcccggacccctcaggatgtagtaactggagttagtcc

SEQ ID NO: 73 - DNA, 42 bp, Artificial Sequence
  Synthetic
ggactaactccagttactacatcctgaggggtccgggcgagc

SEQ ID NO: 74 - DNA, 21 bp, Artificial Sequence
  Synthetic
atccaggtgctgctgagtcca

SEQ ID NO: 75 - DNA, 23 bp, Artificial Sequence
  Synthetic
atccaggtgctgctgagtccact

SEQ ID NO: 76 - DNA, 23 bp, Artificial Sequence
  Synthetic
tgtcatctgtgtcatccaccctg

SEQ ID NO: 77 - DNA, 21 bp, Artificial Sequence
  Synthetic
atccaggtgctgctgagtcca

SEQ ID NO: 78 - DNA, 25 bp, Artificial Sequence
  Synthetic
tgtagacaagaggcaaaatagcgtg

SEQ ID NO: 79 - DNA, 21 bp, Artificial Sequence
  Synthetic
cgccacgtacatgaagtgcag

SEQ ID NO: 80 - DNA, 25 bp, Artificial Sequence
  Synthetic
caagaggcaaaatagcgtgtctttc

SEQ ID NO: 81 - DNA, 24 bp, Artificial Sequence
  Synthetic
ccacgaagaacagaagcacaaagg

SEQ ID NO: 82 - DNA, 24 bp, Artificial Sequence
  Synthetic
gctgccatattaccgaagatggac

SEQ ID NO: 83 - DNA, 23 bp, Artificial Sequence
  Synthetic
attcccacatagaggatgtccca

SEQ ID NO: 84 - DNA, 25 bp, Artificial Sequence
  Synthetic
tgcggttgtgtgtcaagttactacc

SEQ ID NO: 85 - DNA, 23 bp, Artificial Sequence
  Synthetic
ccttccagacattctgcctcctg

SEQ ID NO: 86 - DNA, 25 bp, Artificial Sequence
  Synthetic
gctaactgaaccagcatggtaaggt

SEQ ID NO: 87 - DNA, 23 bp, Artificial Sequence
  Synthetic
gacattctgcctcctgtgtaccc

SEQ ID NO: 88 - DNA, 24 bp, Artificial Sequence
  Synthetic
tgagacctgcctgaatggcgggaa

SEQ ID NO: 89 - DNA, 26 bp, Artificial Sequence
  Synthetic
gcccacgaagaacagaagcacaaagg

SEQ ID NO: 90 - DNA, 23 bp, Artificial Sequence
  Synthetic
acccgagccggatgtgccaagat

SEQ ID NO: 91 - DNA, 23 bp, Artificial Sequence
  Synthetic
gccgccacgtacatgaagtgcag

SEQ ID NO: 92 - DNA, 24 bp, Artificial Sequence
  Synthetic
gatggtgttgctaacaatgatcgc

SEQ ID NO: 93 - DNA, 22 bp, Artificial Sequence
  Synthetic
tgcatccgtgttcttgaagcag

SEQ ID NO: 94 - DNA, 24 bp, Artificial Sequence
  Synthetic
cctgaatggcgggaagtgtgaagc

SEQ ID NO: 95 - DNA, 24 bp, Artificial Sequence
  Synthetic
ctgcaaacaccctgagtgccagtg

SEQ ID NO: 96 - DNA, 22 bp, Artificial Sequence
  Synthetic
tcacccacgagtgtgcctgcct

SEQ ID NO: 97 - DNA, 23 bp, Artificial Sequence
  Synthetic
tccaccgtctgagggaaagctcg

SEQ ID NO: 98 - DNA, 23 bp, Artificial Sequence
  Synthetic
ggtgaggttgacgccgactgcat

SEQ ID NO: 99 - DNA, 25 bp, Artificial Sequence
  Synthetic
gacgatgccaagccagatggtcata

SEQ ID NO: 100 - DNA, 26 bp, Artificial Sequence
  Synthetic
tgatgactgccctaaccacaggtgtc

SEQ ID NO: 101 - DNA, 26 bp, Artificial Sequence
  Synthetic
tggctcctgcttccaaggtacatctg

SEQ ID NO: 102 - DNA, 33 bp, Artificial Sequence
  Synthetic
gaacggtccgaccatgctgctgtcccgcaagcg

SEQ ID NO: 103 - DNA, 28 bp, Artificial Sequence
  Synthetic
gaacggaccgaaggcttgggaaaggaag

SEQ ID NO: 104 - DNA, 24 bp, Artificial Sequence
  Synthetic
tgagacctgcctgaatggcgggaa

SEQ ID NO: 105 - DNA, 26 bp, Artificial Sequence
  Synthetic
gcccacgaagaacagaagcacaaagg

SEQ ID NO: 106 - DNA, 23 bp, Artificial Sequence
  Synthetic
acccgagccggatgtgccaagat

SEQ ID NO: 107 - DNA, 23 bp, Artificial Sequence
  Synthetic
gccgccacgtacatgaagtgcag

SEQ ID NO: 108 - DNA, 35 bp, Artificial Sequence
  Synthetic
gaacggtccgaccatggcaaaacgaaagcgtaagc

SEQ ID NO: 109 - DNA, 31 bp, Artificial Sequence
  Synthetic
caacggaccggatgaccttcatttgttcctc

SEQ ID NO: 110 - DNA, 28 bp, Artificial Sequence
  Synthetic
aaagccgggaaatgcaagcgattctccg

SEQ ID NO: 111 - DNA, 24 bp, Artificial Sequence
  Synthetic
aaagccgggaaatggtgcgtgtgc

SEQ ID NO: 112 - DNA, 29 bp, Artificial Sequence
  Synthetic
gccccctccatgcagcaagcgattctccg

SEQ ID NO: 113 - DNA, 28 bp, Artificial Sequence
  Synthetic
aaaaagaaaaggaggcctatgaacgctc

SEQ ID NO: 114 - DNA, 24 bp, Artificial Sequence
  Synthetic
aaaaagaaaaggaggtaacaaaag

SEQ ID NO: 115 - DNA, 29 bp, Artificial Sequence
  Synthetic
tgtcccctgccacaggcctatgaacgctc

SEQ ID NO: 116 - DNA, 28 bp, Artificial Sequence
  Synthetic
tgtccacactccgggtgtatagcagcat

SEQ ID NO: 117 - DNA, 24 bp, Artificial Sequence
  Synthetic
tgtccacactccgggtaggtcgtt

SEQ ID NO: 118 - DNA, 29 bp, Artificial Sequence
  Synthetic
caccaccacctacaggtgtatagcagcat

SEQ ID NO: 119 - DNA, 28 bp, Artificial Sequence
  Synthetic
cccggacccctcaggatgtagtaactgg

SEQ ID NO: 120 - DNA, 24 bp, Artificial Sequence
  Synthetic
cccggacccctcaggtacacagct

SEQ ID NO: 121 - DNA, 29 bp, Artificial Sequence
  Synthetic
tttttctctttgaaggatgtagtaactgg

SEQ ID NO: 122 - DNA, 27 bp, Artificial Sequence
  Synthetic
ggtgaaaacaaagttgtcggacaagta

SEQ ID NO: 123 - DNA, 23 bp, Artificial Sequence
  Synthetic
ggtgaaaacaaaggtgaggctca

SEQ ID NO: 124 - DNA, 29 bp, Artificial Sequence
  Synthetic
ttgttttcattgcagttgtcggacaagta

SEQ ID NO: 125 - DNA, 28 bp, Artificial Sequence
  Synthetic
tctcagccgcgcgggtgagaccgtggag

SEQ ID NO: 126 - DNA, 24 bp, Artificial Sequence
  Synthetic
tctcagccgcgcgggtgaggggcg

SEQ ID NO: 127 - DNA, 29 bp, Artificial Sequence
  Synthetic
cccccgtgcccgcaggtgagaccgtggag

SEQ ID NO: 128 - DNA, 28 bp, Artificial Sequence
  Synthetic
acggaggcctgcgtgtgagaccgtggag

SEQ ID NO: 129 - DNA, 24 bp, Artificial Sequence
  Synthetic
acggaggcctgcgtgtgagtacca

SEQ ID NO: 130 - DNA, 29 bp, Artificial Sequence
  Synthetic
cccccgtgcccgcaggtgagaccgtggag

SEQ ID NO: 131 - DNA, 28 bp, Artificial Sequence
  Synthetic
tgtccgcgggcctgccggacccagcagg

SEQ ID NO: 132 - DNA, 24 bp, Artificial Sequence
  Synthetic
tgtccgcgggcctggtaagggtgc

SEQ ID NO: 133 - DNA, 29 bp, Artificial Sequence
  Synthetic
tttctctttgcacagccggacccagcagg

SEQ ID NO: 134 - DNA, 28 bp, Artificial Sequence
  Synthetic
acggaggcctgcgtatgcagaggccagg

SEQ ID NO: 135 - DNA, 24 bp, Artificial Sequence
  Synthetic
acggaggcctgcgtgtgagtacca

SEQ ID NO: 136 - DNA, 29 bp, Artificial Sequence
  Synthetic
ttgtttgtttttcagatgcagaggccagg

SEQ ID NO: 137 - DNA, 28 bp, Artificial Sequence
  Synthetic
tgcctgcctgccagcaagccggtgagct

SEQ ID NO: 138 - DNA, 24 bp, Artificial Sequence
  Synthetic
tgcctgcctgccaggtagtgctgc

SEQ ID NO: 139 - DNA, 29 bp, Artificial Sequence
  Synthetic
cgtatttattgacagcaagccggtgagct

SEQ ID NO: 140 - DNA, 28 bp, Artificial Sequence
  Synthetic
aggaggacgaacagctctaaagtctttc

SEQ ID NO: 141 - DNA, 24 bp, Artificial Sequence
  Synthetic
aggaggacgaacaggtgagctgct

SEQ ID NO: 142 - DNA, 29 bp, Artificial Sequence
  Synthetic
cttggcatcccacagctctaaagtctttc

SEQ ID NO: 143 - DNA, 28 bp, Artificial Sequence
  Synthetic
cccacaatggacagtctggccgggacct

SEQ ID NO: 144 - DNA, 24 bp, Artificial Sequence
  Synthetic
cccacaatggacaggtatgtacag

SEQ ID NO: 145 - DNA, 29 bp, Artificial Sequence
  Synthetic
gatgtcttctttcagtctggccgggacct

SEQ ID NO: 146 - DNA, 21 bp, Artificial Sequence
  Synthetic
ttggagttggccctgatgtcc

SEQ ID NO: 147 - DNA, 18 bp, Artificial Sequence
  Synthetic
atctttgggcggtcacag

SEQ ID NO: 148 - DNA, 21 bp, Artificial Sequence
  Synthetic
aacagtggtaaaaacccgtta

(19) United States
(12) Patent Application Publication
(10) Pub. No.: US 2013/0096021 A1
(43) Pub. Date: Apr. 18, 2013

(54) RECURRENT GENE FUSIONS IN BREAST CANCER

(71) Applicants: Arul M. Chinnaiyan, Plymouth, MI (US); Chandan Kumar-Sinha, Ann Arbor, MI (US); Dan Robinson, Ann Arbor, MI (US); Shanker Kalyana-Sundaram, Ann Arbor, MI (US)

(72) Inventors: Arul M. Chinnaiyan, Plymouth, MI (US); Chandan Kumar-Sinha, Ann Arbor, MI (US); Dan Robinson, Ann Arbor, MI (US); Shanker Kalyana-Sundaram, Ann Arbor, MI (US)

(21) Appl. No.: 13/629,188

(22) Filed: Sep. 27, 2012

Related U.S. Application Data
(60) Provisional application No. 61/539,737, filed on Sep. 27, 2011.

(51) Int. Cl. C12Q 1/68; C07K 16/32

(52) U.S. Cl. CPC C12Q 1/6886; C07K 16/32
USPC 506/9; 536/24.31; 536/24.33; 506/16; 435/7.23; 435/6.11; 435/6.12; 435/6.14; 530/387.9

(57) ABSTRACT

The present disclosure relates to compositions and methods for cancer diagnosis, research and therapy, including but not limited to, cancer markers. In particular, the present disclosure relates to gene fusions as diagnostic markers and clinical targets for breast cancer.

This application claims priority to U.S. Provisional Application No. 61/539,737, filed Sep. 27, 2011, which is herein incorporated by reference in its entirety.

### GOVERNMENT SUPPORT

This invention was made with government support under W81XWH-08-1-0110 and W81XWH-09-2-0014 awarded by The Army Medical Research and Materiel Command and CA111275 and CA046952 awarded by the National Institutes of Health. The government has certain rights in the invention.

### FIELD OF THE INVENTION

The present disclosure relates to compositions and methods for cancer diagnosis, research and therapy, including but not limited to, cancer markers. In particular, the present disclosure relates to gene fusions as diagnostic markers and clinical targets for breast cancer.

### BACKGROUND OF THE INVENTION

Breast cancer is the second most common form of cancer among women in the U.S., and the second leading cause of cancer deaths among women. While the 1980s saw a sharp rise in the number of new cases of breast cancer, that number now appears to have stabilized. The drop in the death rate from breast cancer is probably due to the fact that more women are having mammograms. When detected early, the chances for successful treatment of breast cancer are much improved.

Breast cancer, which is highly treatable by surgery, radiation therapy, chemotherapy, and hormonal therapy, is most often curable when detected in early stages. Mammography is the most important screening modality for the early detection of breast cancer. Breast cancer is classified into a variety of sub-types, but only a few of these affect prognosis or selection of therapy. Patient management following initial suspicion of breast cancer generally includes confirmation of the diagnosis, evaluation of stage of disease, and selection of therapy. Diagnosis may be confirmed by aspiration cytology, core needle biopsy with a stereotactic or ultrasound technique for nonpalpable lesions, or incisional or excisional biopsy. At the time the tumor tissue is surgically removed, part of it is processed for determination of ER and PR levels.

Prognosis and selection of therapy are influenced by the age of the patient, stage of the disease, pathologic characteristics of the primary tumor including the presence of tumor necrosis, estrogen-receptor (ER) and progesterone-receptor (PR) levels in the tumor tissue, HER2 overexpression status and measures of proliferative capacity, as well as by menopausal status and general health. Overweight patients may have a poorer prognosis (Bastarrachea et al., Annals of Internal Medicine, 120: 18 [1994]). Prognosis may also vary by race, with blacks, and to a lesser extent Hispanics, having a poorer prognosis than whites (Elledge et al., Journal of the National Cancer Institute 86: 705 [1994]; Edwards et al., Journal of Clinical Oncology 16: 2693 [1998]).

The three major treatments for breast cancer are surgery, radiation, and drug therapy. No treatment fits every patient, and often two or more are required. The choice is determined by many factors, including the age of the patient and her menopausal status, the type of cancer (e.g., ductal vs. lobular), its stage, whether the tumor is hormone-receptive or not, and its level of invasiveness.

Breast cancer treatments are defined as local or systemic. Surgery and radiation are considered local therapies because they directly treat the tumor, breast, lymph nodes, or other specific regions. Drug treatment is called systemic therapy, because its effects are wide spread. Drug therapies include classic chemotherapy drugs, hormone blocking treatment (e.g., aromatase inhibitors, selective estrogen receptor modulators, and estrogen receptor downregulators), and monoclonal antibody treatment (e.g., against HER2). They may be used separately or, most often, in different combinations.

There is a need for additional diagnostic and treatment options, particularly treatments customized to a patient's tumor.

### SUMMARY OF THE INVENTION

The present disclosure relates to compositions and methods for cancer diagnosis, research and therapy, including but not limited to, cancer markers. In particular, the present disclosure relates to gene fusions as diagnostic markers and clinical targets for breast cancer.

For example, in some embodiments, A kit for detecting gene fusions associated with cancer a subject, comprising at least a first gene fusion informative reagent for identification of a gene fusion comprising a 5′ member and a 3′ member, wherein the gene fusion is selected from, for example: a MAST gene fusion (e.g., ZNF700-MAST1, NFIX-MAST1, ARID1A-MAST2, TADA2A-MAST1, or GPBP1L1-MAST2), a NOTCH gene fusion (e.g., SEC16A-NOTCH1, SEC22B-NOTCH2, NOTCH1-GABRR2, NOTCH1-ch9:138722833, NOTCH1-SNHG7, NOTCH2-SEC22B, NOTCH2-ATP1A1, NOTCH2-FBXL20, NOTCH2-MACF1, NOTCH2-MAGI3, NOTCH2-TMEM150C, NOTCH3-VIM), a NOTCH deletion, a FGFR fusion (e.g., FGFR2-ATE1, FGFR2-AFF3FGFR1-ZNF791, FGFR1-WHSC1L1, FGFR2-CCDC6, FGFR2-CASP7, FGFR1-ERLIN2, FGFR1-GPR124, FGFR1-RHOT1, FGFR1-TACC1, FGFR2-NSMCE4A), an ETV6 fusion (e.g., YTHDF2-ETV6, CIT-ETV6, PEX5-ETV6, BCL2L14-ETV6, ETV6-CD70, ETV6-SYN1), GTF2I-ETV7, CTNNA1-JMJD1B or RB1CC1-JAK1. In some embodiments, the reagent is a probe that specifically hybridizes to the fusion junction of the gene fusion, a pair of primers that amplify a fusion junction of the gene fusion (e.g., a first primer that hybridizes to a 5′ member of the gene fusion and second primer that hybridizes to a 3′ member of the gene fusion), an antibody that binds to the fusion junction of a gene fusion polypeptide, a sequencing primer that binds to the gene fusion and generates an extension product that spans the fusion junction of the gene fusion, or a pair of probes wherein the first probe hybridizes to a 5′ member of the gene fusion and the second probe hybridizes to a 3′ member of the gene fusion gene. In some embodiments, the reagent is labeled. In some embodiments, the cancer is breast cancer.

In some embodiments, the present invention further provides a method for identifying cancer (e.g., breast cancer) in a patient comprising: a) contacting a biological sample from a subject with a nucleic acid or polypeptide detection assay comprising at least a first gene fusion informative reagent for identification of a gene fusion comprising a 5′ member and a 3′ member, wherein the gene fusion is selected from, for example: a MAST gene fusion (e.g., ZNF700-MAST1, NFIX-MAST1, ARID1A-MAST2, TADA2A-MAST1, or GPBP1L1-MAST2), a NOTCH gene fusion (e.g., SEC16A-NOTCH1, SEC22B-NOTCH2, NOTCH1-GABRR2, NOTCH1-ch9:138722833, NOTCH1-SNHG7, NOTCH2-SEC22B, NOTCH2-ATP1A1, NOTCH2-FBXL20, NOTCH2-MACF1, NOTCH2-MAGI3, NOTCH2-TMEM150C, NOTCH3-VIM), a NOTCH deletion, a FGFR fusion (e.g., FGFR2-ATE1, FGFR2-AFF3FGFR1-ZNF791, FGFR1-WHSC1L1, FGFR2-CCDC6, FGFR2-CASP7, FGFR1-ERLIN2, FGFR1-GPR124, FGFR1-RHOT1, FGFR1-TACC1, FGFR2-NSMCE4A), an ETV6 fusion (e.g., YTHDF2-ETV6, CIT-ETV6, PEX5-ETV6, BCL2L14-ETV6, ETV6-CD70, ETV6-SYN1), GTF2I-ETV7, CTNNA1-JMJD1B or RB1CC1-JAK1; and b) identifying cancer (e.g., breast cancer) in said subject when the gene fusion is present in the sample. In some embodiments, the sample is, for example, tissue, blood, plasma, serum, cells or tissues. In some embodiments, the method further comprises the step of determining a treatment course of action based on the presence or absence of the gene fusion in the sample. For example, in some embodiments, the treatment course of action comprises administration of an inhibitor that targets a member of the gene fusion when the gene fusion is present in the sample.

Additional embodiments of the present disclosure are provided in the description and examples below.

### DESCRIPTION OF THE FIGURES

FIG. 1 shows discovery of the MAST kinase and Notch gene fusions in breast cancer identified by paired-end transcriptome sequencing. (a) Diagram of MAST family gene fusions. ZNF700-MAST1 in BrCa00001, NFIX-MAST1 in BrCa10017, TADA2AMAST1 in BrCa10038, ARID1A-MAST2 in the breast cancer cell line MDA-MB-468, and GPBP1L1-MAST2 in BrCa10039 are shown. (b) Diagram of Notch family gene fusions. SEC16A-NOTCH1 in HCC2218, NOTCH1 Exon2-28 in HCC1599, and SEC22BNOTCH2 in HCC1187 are shown.

FIG. 2 shows experimental validations of MAST gene fusions in the index breast cancer samples. (a) Expression of ZNF700-MAST1 gene fusion in breast cancer tissue BrCa00001, NFIX-MAST1 in BrCa10017, TADA2A-MAST1 fusion in BrCa10038, and ARID1A-MAST2 fusion in MDA-MB-468 validated by RT-PCR normalized against glyceraldehyde 6-phosphate dehydrogenase (GAPDH) values in each sample. (b) Western blot showing a higher molecular weight band above MAST2, corresponding to the fusion protein ARID1A-MAST2, specifically observed in the index breast cancer cell line MDA-MB-468. (c) Schematic representation of functional domains retained in the putative chimeric proteins involving MAST1 and (d) involving MAST2.

FIG. 3 shows functional characterization of MAST fusion genes. (a) Percentage confluency over a time course was measured using the Incucyte system for polyclonal populations of HMEC-TERT cells over-expressing full length MAST2, allelic MAST1 (truncated ORF from ZNF700-MAST1 transcript in BrCa00001) and empty vector control. (b) Wound healing assay using the Incucyte system. (c) Histogram showing growth of HMEC-TERT cells stably over-expressing MAST1, MAST2 or vector control on chicken chorionic allantoic membrane (CAM) assay. (d) Graphical representation of cell proliferation assay showing cell numbers (y-axis) over the indicated time course (x-axis) with MAST2 knockdown using three independent siRNAs and one shRNA construct in MDA-MB-468 cells harboring the ARID1A-MAST2 fusion (left) and in fusion negative HMEC-TERT and BT-483 cells, as indicated (right). (e) Histogram representation of colony formation assay with MDA-MB-468 cells treated with MAST2 specific shRNA or control-scrambled sequence-shRNA. (f) Tumor growth in immunodeficient mice implanted with MDA-MB-468 cells transfected with MAST2-shRNA or scrambled control shRNA.

FIG. 4 shows identification and characterization of novel Notch gene aberrations in breast carcinomas. (a) Detection of novel Notch transcripts by quantitative RT-PCR. (b) Schematic presentation of the predicted protein structures of the three aberrant Notch genes. (c) Notch reporter activities are elevated in Notch fusion index lines. (d) Western blot analysis of NOTCH1-NICD expression. (e) Activation of Notch signaling pathway in 293T cells by transient Notch expression. (f) Notch fusion alleles induce morphological change when expressed in benign TERTHME1 (g) Activation of Notch signaling pathway in TERTHME1 cells stably expressing Notch fusions.

FIG. 5 shows that the γ-secretase inhibitor DAPT blocked Notch-dependent cell proliferation. (a) Inhibition of the Notch signaling pathway by DAPT. (b) Reduction of NICD production after DAPT treatment. (c) Inhibition of cell proliferation by DAPT. (d) Diminished expression of Notch target genes by DAPT. (e) Inhibition of tumor growth by DAPT in a mouse xenograft model.

FIG. 6 shows that recurrent loci of amplifications are hotspots of gene fusions in breast cancer. (a) Histograms of number of gene fusions in individual samples with respect to their association with loci of genomic amplifications. (b) Circos plot presentation of chromosomal locations of gene fusions in breast cancer cell line BT-474 (left) and MCF7 (right).

FIG. 7 shows schematic presentation of exon splice junctions identified in the MAST family and Notch family gene fusions.

FIG. 8 shows identification of Notch gene aberrations in breast carcinomas. (a) Exon expression imbalance of NOTCH1 gene expression in the index cell lines HCC2218 and HCC1599, compared to wild type NOTCH1 expression in the normal cell line MCF10F.

FIG. 9 shows immunoblot analysis of HEK293 cells overexpressing (a) fusion allelic MAST1 using anti-V5 antibody and (b) full length MAST2 using anti-DDK antibody. (c) qPCR validation of TERT-HME1 cells overexpressing fusion MAST1 and FL-MAST2. (d) Immunoblot analysis of TERT-HME1 cells overexpressing fusion MAST1 and (e) FL-MAST2 proteins. (f) Cell proliferation assay of TERT-HME1 cells overexpressing fusion MAST1, FL-MAST2, and vector control. (g) Wound healing assay using the Incucyte system. (h) In vivo chicken chorioallantoic membrane assay of TERT-HME1 cells overexpressing fusion MAST1 or FL-MAST2 compared to vector control.

FIG. 10 shows (a) qPCR validation of MAST2 and ARID1A-MAST2 knockdown using MAST2 siRNAs in MDA-MB-468 cells. qPCR validation of MAST2 knockdown (b) in fusion negative BT-483 cells (c) in H16N2 cells (d) in HMEC-TERT cells. Validation of MAST2 knockdown in MDA-MB-468 cells by (e) qPCR and (f) anti-MAST2 immunoblot.

FIG. 11 shows (a) Flow cytometric analysis of MDA-MB-468 cells treated with scrambled shRNA or MAST2 shRNA. (b) Percentage distribution of the MDA-MB-468 cells in different phases of the cell cycle after treatment with either the scrambled shRNA or MAST2 shRNA. (c) Chicken chorioallantoic membrane assay showing tumor weight of MDA-MB-468 cells treated with either scrambled shRNA or MAST2 shRNA.

FIG. 12 shows notch gene fusions identified by paired-end transcriptome sequencing in breast carcinoma samples. (a) Schematic presentation of Notch fusions identified in breast carcinoma. The SEC16A-NOTCH1 in HCC22218, NOTCH1 internal deletion in HCC1599, SEC22B-NOTCH2 in HCC1187, NOTCH1-GABBR2 in BT-20, NOTCH1-SNHG7 in breast tumor BrCa10033, NOTCH1-chr9:138722833 in breast tumor BrCa10002, and NOTCH2-SEC22B in HCC38 are shown. (b) Validation of the Notch fusions by SYBR Green-QPCR. Expression levels of the fusion transcript normalized using GAPDH levels are shown for each index case and a panel of other breast carcinomas.

FIG. 13 shows a diagram of molecular steps involved in Notch pathway activation.

FIG. 14 shows (a) A flowchart of the transcriptome analysis and (b) a summary of the number of gene fusions discovered in this study.

FIG. 15 shows (a) qPCR analysis of ARID1A-MAST2 fusion and ARID1A transcripts in MDAMB-468 cells after treatment with ARID1A-MAST2 fusion specific siRNAs. Cell proliferation rates of (b) MDA-MB-468, (c) benign TERT-HME1 and (d) MDA-MB-453 cells upon treatment with ARID1A-MAST2 fusion specific siRNAs. (e) Immunoblot analysis of MAST2 levels in MDA-MB-453 (fusion negative) cells treated with ARID1A-MAST2 fusion siRNAs.

FIG. 16 shows Immunoblot analysis of signaling molecules (pAkt and pERK) in (a) multiple MAST1 fusion and (b) MAST2 fusion overexpressing TERT-HME1 cells compared to empty vector control cells. (c) Immunoblot analysis of a panel of signaling molecules in MDA-MB-468 cells upon treatment with ARID1A-MAST2 fusion specific siRNAs.

FIG. 17 a-d shows FGFR gene fusions in breast cancer.

FIG. 18 shows FGFR gene fusions in breast cancer.

FIG. 19 shows ETV6 gene fusions in breast cancer.

FIG. 20 shows ETV6 gene fusions in breast cancer.

FIG. 21 shows ETV6 gene fusions in breast cancer.

FIG. 22 shows CTNNA1-JMJD1B fusions in breast cancer.

FIG. 23 shows CTNNA1-JMJD1B fusions in breast cancer.

FIG. 24 shows RB1CC1-JAK1 fusions in breast cancer.

FIG. 25 shows RB1CC1-JAK1 fusions in breast cancer.

FIG. 26 shows RB1CC1-JAK1 fusions in breast cancer.

### DEFINITIONS

Unless defined otherwise, all terms of art, notations and other scientific terms or terminology used herein have the same meaning as is commonly understood by one of ordinary skill in the art to which this disclosure belongs. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the term “gene fusion” refers to a chimeric genomic DNA, a chimeric messenger RNA, a truncated protein or a chimeric protein resulting from the fusion of at least a portion of a first gene to at least a portion of a second gene. In some embodiments, gene fusions involve internal deletions of genomic DNA within a single gene (e.g., no second gene is involved in the fusion). The gene fusion need not include entire genes or exons of genes.

As used herein, the term “gene upregulated in cancer” refers to a gene that is expressed (e.g., mRNA or protein expression) at a higher level in cancer (e.g., breast cancer) relative to the level in other tissue. In this context, “other tissue” may refer to, for example, tissues from different organs in the same subject or to normal tissues of the same or different type. In some embodiments, genes upregulated in cancer are expressed at a level between at least 10% to 300% higher than the level of expression in other tissue. For example, genes upregulated in cancer are frequently expressed at a level preferably at least 25%, at least 50%, at least 100%, at least 200%, or at least 300% higher than the level of expression in other tissue.

As used herein, the term “gene upregulated in breast tissue” refers to a gene that is expressed (e.g., mRNA or protein expression) at a higher level in breast tissue relative to the level in other tissue. In some embodiments, genes upregulated in breast tissue are expressed at a level between at least 10% to 300%. For example, genes upregulated in cancer are frequently expressed at a level preferably at least 25%, at least 50%, at least 100%, at least 200%, or at least 300% higher than the level of expression in other tissues. In some embodiments, genes upregulated in breast tissue are exclusively expressed in breast tissue.

As used herein, the term “transcriptional regulatory region” refers to the region of a gene comprising sequences that modulate (e.g., upregulate or downregulate) expression of the gene. In some embodiments, the transcriptional regulatory region of a gene comprises a non-coding upstream sequence of a gene, also called the 5′ untranslated region (5′UTR). In other embodiments, the transcriptional regulatory region contains sequences located within the coding region of a gene or within an intron (e.g., enhancers).

As used herein, the terms “detect”, “detecting” or “detection” may describe either the general act of discovering or discerning or the specific observation of a detectably labeled composition.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “probe” refers to an oligonucleotide, whether occurring naturally as in a purified restriction digest or produced synthetically, recombinantly or by PCR amplification, which is capable of hybridizing to at least a portion of another oligonucleotide of interest. A probe may be single-stranded or double-stranded. Probes are useful in the detection, identification and isolation of particular gene sequences. It is contemplated that any probe used in methods of the present disclosure will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the methods or reagents of the present disclosure be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. An isolated nucleic acid is present in a form or setting that is different from that in which it is found in nature. In contrast, non-isolated nucleic acids are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the nucleic acid, oligonucleotide or polynucleotide often will contain, at a minimum, the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

Provided herein are compositions and methods for cancer diagnosis, research and therapy, including but not limited to, cancer markers. In particular, the present disclosure relates to gene fusions as diagnostic markers and clinical targets for breast cancer.

Recurrent gene fusions and translocations have long been associated with hematologic malignancies and rare soft tissue tumors as driving genetic lesions (Delattre, O. et al. Nature 359, 162-5 (1992); Nowell et al., J Natl Cancer Inst 25, 85-109 (1960); Rowley, J. D. Annu Rev Genet 32, 495-519 (1998)). Over the last few years, it is becoming apparent that these genetic rearrangements are also found in common solid tumors including a large subset of prostate cancers (Kumar-Sinha et al., Nat Rev Cancer 8, 497-511 (2008); Tomlins, S. A. et al. Science 310, 644-8 (2005)) and smaller subsets of lung cancer, among others (Prensner, J. R. & Chinnaiyan, Curr Opin Genet Dev 19, 82-91 (2009)). A number of these gene fusions are targetable including BCR-ABL in chronic myelogenous leukemia (Druker, B. J. Translation of the Philadelphia chromosome into therapy for CML. Blood 112, 4808-17 (2008)), ALK gene fusions in non-small cell lung cancer (Perner, S. et al. Neoplasia 10, 298-302 (2008); Soda, M. et al. Nature 448, 561-6 (2007)) RET in papillary thyroid cancer (Grieco, M. et al. Cell 60, 557-63 (1990)), and RAF family fusions in prostate cancer and other solid tumors (Palanisamy, N. et al. Nat Med 16, 793-8 (2010)).

Breast cancer is a heterogeneous disease with several morphologic and molecular subtypes. Experiments conducted during the course of development of embodiments of the present invention identified gene fusions in breast cancer cell lines and tissues. Individual samples often harbored multiple rearrangements, with amplicons being a hot-spot for gene fusion events. Two novel classes of recurrent gene rearrangement in breast cancer involving microtubule associated serine threonine (MAST) kinases and Notch family genes were identified.

Discovery of the genetic aberrations contributing to the development of breast cancer has increased greatly in the past decades, beginning with the discovery of amplification of the HER2 locus in a subset of cases (Slamon, D. J. et al. Science 235, 177-82 (1987)). Breast cancer can be classified into subtypes as estrogen/progesterone receptor positive, HER2 amplification positive, or triple negative, based on expression of these three genes. Triple negative breast carcinoma in particular, lacks detailed molecular characterization (Foulkes et al., N Engl J Med 363, 1938-48 (2010); Sotiriou et al., N Engl J Med 360, 790-800 (2009)). Experiments conducted during the development of embodiments of the present invention identified functional gene fusions involving NOTCH1 and NOTCH2 in estrogen receptor (ER) negative breast carcinomas (Table 1).

The gene fusions in breast cancer involving MAST kinases and the Notch family of transcription factors represent novel classes of functionally recurrent gene fusions with therapeutic implications. MAST kinase and Notch gene rearrangements are mutually exclusive aberrations, and together, may represent up to 8-10% of breast cancers with a particular enrichment in ER negative disease. MAST1 expression has been associated with resistance to the anti-cancer drug 5-fluorouracil (5-FU) (De Angelis et al., Mol Cancer 5, 20 (2006)). In a recent study of genetic variation in mitotic kinases associated with breast cancer risk, identified common haplotypes of MAST2 to be significantly associated with breast cancer risk (P=0.04) (Wang, X. et al. Breast Cancer Res Treat 119, 453-62 (2009)). Functionally, MAST2 has been linked with the dystrophin/utrophin network of microtubule filaments via the syntrophins. MAST2 has also been shown to act as a scaffolding protein for TRAF6, regulating its activity, including inhibition of NF-κB, regulating cellular inflammatory responses (Xiong et al., J Biol Chem 279, 43675-83 (2004)). The tumor suppressor phosphatase PTEN has been shown to interact with the PDZ domain of MAST2 and related serine/threonine kinases (Valiente, M. et al. J Biol Chem 280, 28936-43 (2005)), indicating regulatory networks impacted by MAST genes.

The involvement of aberrant Notch gene function in human cancer was first reported as rare gene fusions in T-cell acute lymphoblastic leukemia (T-ALL) (Ellisen, L. W. et al. Cell 66, 649-61 (1991)). Later studies revealed activating point mutations in NOTCH1 in a majority of T-ALL cases (Grabher et al., Nat Rev Cancer 6, 347-59 (2006)), however mutations of this type have not been found in breast carcinoma.

The target genes of the Notch pathway depend critically on the context of Notch activation (Radtke, F. & Raj, K. Nat Rev Cancer 3, 756-67 (2003)). It has been shown that the phenotypic effects of Notch in mammary epithelial cells vary with dose (Mazzone, M. et al. Proc Natl Acad Sci USA 107, 5012-7 (2010)). Different arrangements of Notch responsive elements in promoters also modulate the effects of Notch activation in a dose dependent manner. The breast carcinoma cell lines investigated herein exhibit dependence on the resulting effects of NOTCH1 activation.

GSIs and other Notch inhibitors, as well as MAST-kinase specific inhibitors or the currently available serine/threonine kinase inhibitors find use in breast cancer therapy (e.g., against cancers expressing the fusions).

### I. Gene Fusions

The present disclosure identifies recurrent gene fusions indicative of cancer (e.g., breast cancer). In some embodiments, the gene fusions are the result of a chromosomal rearrangement of a first and second gene resulting in a gene fusion. Example gene fusions include, but are not limited to, a MAST gene fusion (e.g., zinc finger protein 700 (ZNF700)-microtubule associated serine/threonine kinase 1 (MAST1), nuclear factor I/X (NFIX)-MAST1, AT rich interactive domain 1A (ARID1A)-microtubule associated serine/threonine kinase 2 (MAST2), transcriptional adaptor 2A (TADA2A)-MAST1, GC-rich promoter binding protein 1-like 1 (GPBP1L1)-MAST2), a NOTCH gene fusion (e.g., SEC16 homolog A (SEC16A)-NOTCH1, SEC22 vesicle trafficking protein homolog B (SEC22B)-NOTCH2, NOTCH1-gamma-aminobutyric acid (GABA) A receptor, rho 2 (GABRR2), NOTCH1-ch9:138722833, NOTCH1-small nucleolar RNA host gene 7 (SNHG7), NOTCH2-SEC22B, NOTCH2-ATPase, Na+/K+ transporting, alpha 1 polypeptide (ATP1A1), NOTCH2-F-box and leucine-rich repeat protein 20 (FBXL20), NOTCH2-microtubule-actin crosslinking factor 1 (MACF1), NOTCH2-membrane associated guanylate kinase, WW and PDZ domain containing 3 (MAGI3), NOTCH2-transmembrane protein 150C (TMEM150C), NOTCH3-vimentin (VIM)), a NOTCH deletion, a FGFR fusion (e.g., fibroblast growth factor receptor 2 (FGFR2)-arginyltransferase 1 (ATE1), FGFR2-AF4/FMR2 family, member 3 (AFF3), FGFR1-zinc finger protein 791 (ZNF791), FGFR1-Wolf-Hirschhorn syndrome candidate 1-like 1 (WHSC1L1), FGFR2-coiled-coil domain containing 6 (CCDC6), FGFR2-caspase 7, apoptosis-related cysteine peptidase (CASP7), FGFR1-ER lipid raft associated 2 (ERLIN2), FGFR1-G protein-coupled receptor 124 (GPR124), FGFR1-ras homolog gene family, member T1 (RHOT1), FGFR1-transforming, acidic coiled-coil containing protein 1 (TACC1), FGFR2-non-SMC element 4 homolog A (NSMCE4A)), an ETV6 fusion (e.g., YTH domain family, member 2 (YTHDF2)-ets variant 6 (ETV6), citron (rho-interacting, serine/threonine kinase 21) (CIT)-ETV6, peroxisomal biogenesis factor 5 (PEX5)-ETV6, BCL2-like 14 (apoptosis facilitator) (BCL2L14)-ETV6, ETV6-CD70, ETV6-synapsin I (SYN1)), general transcription factor IIi (GTF2I)-ETV7, catenin (cadherin-associated protein), alpha 1, 102 kDa (CTNNA1)-jumonji domain containing 1B (JMJD1B) or

RB1-inducible coiled-coil 1 (RB1CC1)-Janus kinase 1 (JAK1).

In some embodiments, the 5′ fusion partner is a transcriptional region of a gene (e.g., ZNF700, NFIX, ARIDIA, TADA2A, GPB1L1, SEC16A, a NOTCH kinase and SEC22B).

In some embodiments, the 3′ fusion partner is a kinase (e.g., a MAST or NOTCH family kinase). In some embodiments, the fusion comprises funcational kinase domain(s) of the kinase. In some embodiments, the 3′ fusion partner is, for example, GABBR2, chr9: 138722833, SNHG7 or SEC22B. In some embodiments, gene fusions result in overexpression of the NOTCH or MAST kinase, for example, by the association of a non-native promoter, driving aberrant expression of NOTCH or MAST.

In some embodiments, fusions comprise internal NOTCH fusions (e.g., due to a deletion of NOTCH genomic DNA without a fusion partner).

MAST kinase family genes (MAST1-4, and MAST-like) are characterized by the presence of a serine/threonine kinase domain and a PDZ domain, involved in protein scaffolding and interaction with other proteins (Garland et al., Brain Res 1195, 12-9 (2008)). MAST1 and MAST2 are widely expressed in diverse tissues including brain, heart, liver, lung, kidney, and testis, while MAST3 and MAST4 show more restricted expression in several tissues and MAST-like is predominantly expressed in heart and testis (Garland et al., supra).

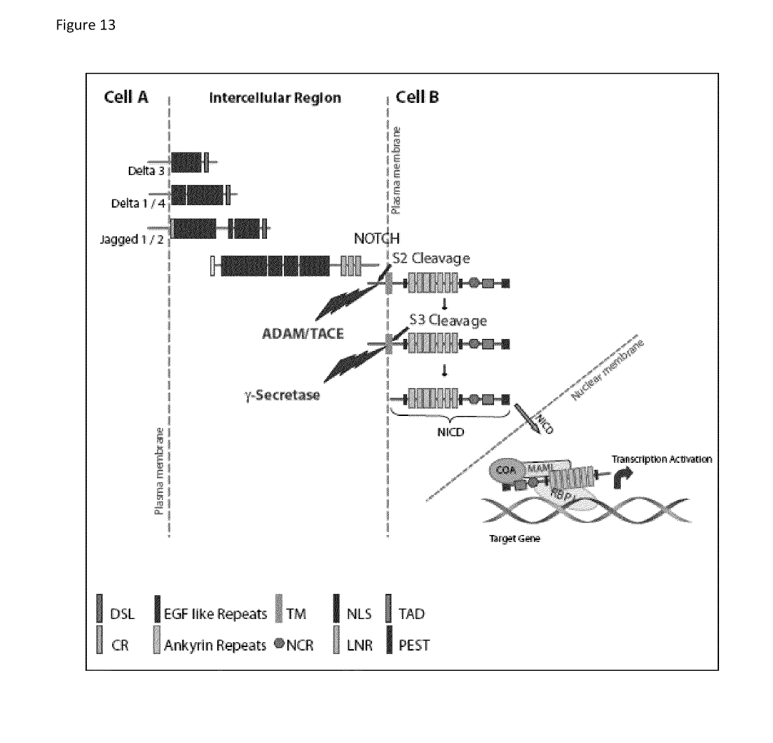

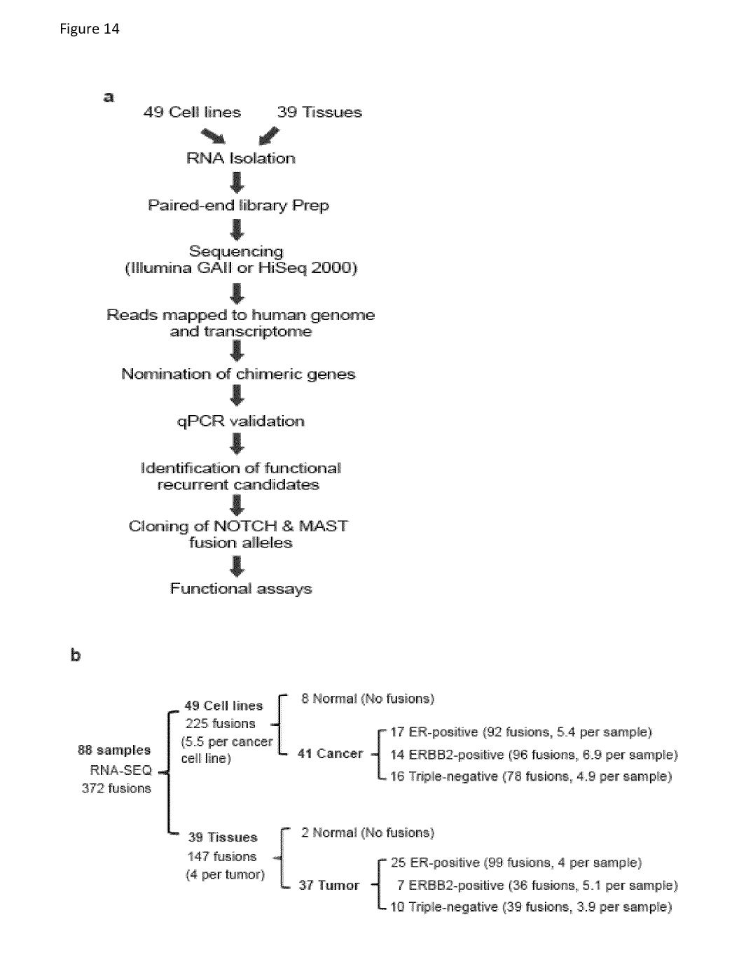

The Notch family of signaling molecules is widely conserved in metazoans and is composed of four members in the human genome. Notch signaling between adjoining cells affects diverse functions including differentiation, proliferation, and self-renewal (Bolos et al., Endocr Rev 28, 339-63 (2007)). The pleiotropic effects of Notch pathway activity are particularly context and dosage dependent (Mazzone, M. et al. Proc Natl Acad Sci USA 107, 5012-7 (2010); Radtke et al., Nat Rev Cancer 3, 756-67 (2003)). The canonical Notch pathway is illustrated in FIG. 13. Following ligand binding, cleavage of Notch proteins by ADAM type proteases at the S2 site is followed by cleavage by γ-secretase at the S3 site, releasing the Notch intracellular domain (NICD) to translocate to the nucleus (Kopan, R. & Ilagan, M. X. Cell 137, 216-33 (2009)). There, NICD interacts with the DNA binding protein RBPJ and recruits transcriptional co-activators, including members of the Mastermind like family (MAML), affecting expression of target genes. Mutations in Notch family genes have wide ranging developmental effects and have been found in a significant percentage of human T-cell acute lymphocytic leukemia (T-ALL) (Demarest et al., Oncogene 27, 5082-91 (2008)). Furthermore, several therapies targeting the Notch pathway in cancer are under late stage clinical investigation (Rizzo, P. et al. Oncogene 27, 5124-31 (2008); Takebe et al., Nat Rev Clin Oncol 8, 97-106 (2011); Wei, P. et al. Mol Cancer Ther 9, 1618-28 (2010)).

### II. Antibodies

The gene fusion proteins of the present disclosure, including fragments, derivatives and analogs thereof, may be used as immunogens to produce antibodies having use in the diagnostic, screening, research, and therapeutic methods described below. The antibodies may be polyclonal or monoclonal, chimeric, humanized, single chain, Fv or Fab fragments. Various procedures known to those of ordinary skill in the art may be used for the production and labeling of such antibodies and fragments. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). Antibodies or fragments exploiting the differences between the truncated or chimeric protein resulting from a gene fusion and their respective native proteins are particularly preferred (e.g., the antibody preferentially binds to the protein expressed by the gene fusion relative to its binding to the protein generated by the non-fusion gene(s)).

### III. Diagnostic and Screening Applications

The gene fusions described herein may be detectable as DNA, RNA or protein. Initially, the gene fusion is detectable as a chromosomal rearrangement of genomic DNA having a 5′ portion from a first gene and a 3′ portion from a second. Once transcribed, the gene fusion may be detectable as a chimeric mRNA having a 5′ portion from a first gene and a 3′ portion from a second gene or a chimeric mRNA with a deletion of mRNA. Once translated, the gene fusion may be detectable as fusion of a 5′ portion from a first protein and a 3′ portion from a second protein or a truncated version of a first or second protein. The truncated or fusion proteins may differ from their respective native proteins in amino acid sequence, post-translational processing and/or secondary, tertiary or quaternary structure. Such differences, if present, can be used to identify the presence of the gene fusion. Specific methods of detection are described in more detail below.

The present disclosure provides DNA, RNA and protein based diagnostic, prognostic and screening methods that either directly or indirectly detect the gene fusions. The present disclosure also provides compositions and kits for diagnostic and screening purposes.

The diagnostic and screening methods of the present disclosure may be qualitative or quantitative. Quantitative methods may be used, for example, to discriminate between indolent and aggressive cancers via a cutoff or threshold level. Where applicable, qualitative or quantitative methods of embodiments of the disclosure include amplification of a target, a signal or an intermediary (e.g., a universal primer).

An initial assay may confirm the presence of a gene fusion but not identify the specific fusion. A secondary assay may then be performed to determine the identity of the particular fusion, if desired. The second assay may use a different detection technology than the initial assay.

The gene fusions may be detected along with other markers in a multiplex or panel format. Markers are selected for their predictive value alone or in combination with the gene fusions. Exemplary breast cancer markers include, but are not limited to those described in U.S. Pat. No. 5,622,829, U.S. Pat. No. 5,720,937, U.S. Pat. No. 6,294,349, each of which is herein incorporated by reference in its entirety. Markers for other cancers, diseases, infections, and metabolic conditions are also contemplated for inclusion in a multiplex or panel format.

The diagnostic methods of the present disclosure may also be modified with reference to data correlating particular gene fusions with the stage, aggressiveness or progression of the disease or the presence or risk of metastasis. Ultimately, the information provided will assist a physician in choosing the best course of treatment for a particular patient.

A. Sample

Any sample suspected of containing the gene fusions may be tested according to the methods of the present disclosure. By way of non-limiting example, the sample may be tissue (e.g., a breast biopsy sample or a tissue sample obtained by mastectomy), blood, cell secretions or a fraction thereof (e.g., plasma, serum, exosomes, etc.).

The patient sample typically involves preliminary processing designed to isolate or enrich the sample for the gene fusion(s) or cells that contain the gene fusion(s). A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

B. DNA and RNA Detection

The gene fusions of the present disclosure may be detected as chromosomal rearrangements of genomic DNA or chimeric mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing, or high throughput sequencing methods. The present disclosure is not intended to be limited to any particular methods of sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

a. FISH

In some embodiments, fusion sequences are detected using fluorescence in situ hybridization (FISH). The preferred FISH assays for methods of embodiments of the present disclosure utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

b. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Southern and Northern blotting may be used to detect specific DNA or RNA sequences, respectively. In these techniques DNA or RNA is extracted from a sample, fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Pat. No. 7,374,885 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified gene fusion nucleic acids can be detected by any conventional means. For example, the gene fusions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174; Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in method of embodiments of the present disclosure. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products methods of embodiments of the present disclosure. See, e.g., U.S. Pat. No. 5,814,447 (herein incorporated by reference in its entirety).

C. Protein Detection

The gene fusions of the present disclosure may be detected as truncated or chimeric proteins using a variety of protein techniques known to those of ordinary skill in the art, including but not limited to: protein sequencing and immunoassays.

1. Sequencing

Illustrative non-limiting examples of protein sequencing techniques include, but are not limited to, mass spectrometry and Edman degradation.

Mass spectrometry can, in principle, sequence any size protein. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction (see, e.g., Edman, Acta Chem. Scand. 4:283-93 (1950)), the peptide to be sequenced is adsorbed onto a solid surface (e.g., a glass fiber coated with polybrene). Though there are various well known modifications to this procedure (including automated modifications), one exemplary method involves the use of the Edman reagent, phenylisothiocyanate (PITC), which is added, together with a mildly basic buffer solution of 12% trimethylamine, to an adsorbed peptide, and which reacts with the amine group of the N-terminal amino acid of the adsorbed peptide. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about or over 98%, which allows about 50 amino acids to be reliably determined.

2. Immunoassays

Illustrative non-limiting examples of immunoassays include, but are not limited to: immunoprecipitation; Western blot; ELISA; immunohistochemistry; immunocytochemistry; immunochromatography; flow cytometry; and, immuno-PCR. Polyclonal or monoclonal antibodies detectably labeled using various techniques known to those of ordinary skill in the art (e.g., colorimetric, fluorescent, chemiluminescent or radioactive labels) are suitable for use in the immunoassays.

Immunoprecipitation is the technique of precipitating an antigen out of solution using an antibody specific to that antigen. The process can be used to identify proteins or protein complexes present in cell extracts by targeting a specific protein or a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and optionally sorting microscopic particles or cells suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

D. Data Analysis

In some embodiments, a computer-based analysis program is used to translate the raw data generated by the detection assay (e.g., the presence, absence, or amount of a given gene fusion or other markers) into data of predictive value for a clinician. The clinician can access the predictive data using any suitable means. Thus, in some preferred embodiments, the present disclosure provides the further benefit that the clinician, who may not be specifically trained in genetics or molecular biology, need not understand the raw data. The data is can be presented directly to the clinician in its most useful form. The clinician is may then be then able to immediately utilize the information in order to optimize the care of the subject.

The present disclosure contemplates any method capable of receiving, processing, and transmitting the information to and from laboratories conducting the assays, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data may then be prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose, for example, further or altered intervention or counseling based on the results. In some embodiments, the data is used for research use. For example, the data may be used to further optimize the inclusion or elimination of markers as useful indicators of a particular condition or stage of disease.

E. In Vivo Imaging

The gene fusions of the present disclosure may also be detected using in vivo imaging techniques, including but not limited to: radionuclide imaging; positron emission tomography (PET); computerized axial tomography, X-ray or magnetic resonance imaging methods, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present disclosure are described below.

The in vivo imaging methods of the present disclosure are useful in the diagnosis of cancers that express the cancer markers of the present invention (e.g., breast cancer). In vivo imaging is used to visualize the presence of a marker indicative of the cancer. Such techniques allow for diagnosis without the use of an unpleasant biopsy. The in vivo imaging methods of the present disclosure are also useful for providing prognoses to cancer patients. For example, the presence of a marker indicative of cancers likely to metastasize can be detected. The in vivo imaging methods of the present disclosure can further be used to detect metastatic cancers in other parts of the body.

In some embodiments, reagents (e.g., antibodies) specific for the gene fusions of the present disclosure are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a gene fusion of the present disclosure). When active, it leads to a reaction that emits light. A CCD camera and software is used to capture the image and analyze it.

F. Compositions & Kits

Any of these compositions, alone or in combination with other compositions of the present disclosure, may be provided in the form of a kit. For example, the single labeled probe and pair of amplification oligonucleotides may be provided in a kit for the amplification and detection of gene fusions of the present invention. Kits may further comprise appropriate controls and/or detection reagents. The probe and antibody compositions of the present disclosure may also be provided in the form of an array.

Compositions for use in the diagnostic methods of the present invention include, but are not limited to, probes, amplification oligonucleotides, and antibodies. Particularly preferred compositions detect a product only when a first gene fuses to a second gene. These compositions include: a single labeled probe comprising a sequence that hybridizes to the junction at which a 5′ portion from a first gene fuses to a 3′ portion from a second gene (i.e., spans the gene fusion junction); a pair of amplification oligonucleotides wherein the first amplification oligonucleotide comprises a sequence that hybridizes to a transcriptional regulatory region of a 5′ portion from a first gene fuses to a 3′ portion from a second gene; an antibody to an amino-terminally truncated protein resulting from a fusion of a first protein to a second gene; or, an antibody to a chimeric protein having an amino-terminal portion from a first gene and a carboxy-terminal portion from a second gene. Other useful compositions, however, include: a pair of labeled probes wherein the first labeled probe comprises a sequence that hybridizes to a transcriptional regulatory region of a first gene and the second labeled probe comprises a sequence that hybridizes to a second gene, probes and primers that span the fusion junction of a fusion generated by an internal deletion and antibodies that bind to amino acid sequences generated by internal deletions.

### IV. Companion Diagnostics

In some embodiments, the present disclosure provides compositions and methods for determining a treatment course of action in response to a subject's gene fusion status. For example, screening for NOTCH or MAST family kinase fusions is useful in identifying people with cancer who benefit from treatment with NOTCH or MAST kinase inhibitors. Individuals found to a have a gene fusions that comprises a NOTCH or MAST family member gene fusion are then treated with a NOTCH or MAST inhibitor, respectively.

The present disclosure is not limited to a particular NOTCH or MAST pathway inhibitor. NOTCH and MAST kinase inhibitors are known in the art. In some embodiments, inhibitors are antisense oligonucleotides, siRNA, antibodies and small molecules. Exemplary small molecule inhibitors include, but are not limited to, GSIs and other Notch inhibitors, as well as MAST-kinase specific inhibitors or the currently available serine/threonine kinase inhibitors. Examples include, but are not limited to, γ-secretase inhibitors (e.g., IL-X (cbz-IL-CHO), tripeptide γ-secretase inhibitor (z-Leu-leu-Nle-CHO), dipeptide γ-secretase inhibitor N—[N-(3,5-difluorophenacetyl)-L-alanyl]-S-phenylglycine t-butyl ester (DAPT), dibenzazepine), MK0752 (developed by Merck, Whitehouse Station, N.J.).

In other embodiments, FGF fusions are targeted by, for example, R3Mab, Palifermin or Kepivance (Amgen inc).

### V. Drug Screening Applications

In some embodiments, the present disclosure provides drug screening assays (e.g., to screen for anticancer drugs). In some embodiments, the screening methods utilize cancer markers described herein. For example, in some embodiments, provided herein are methods of screening for compounds that alter (e.g., decrease) the expression of gene fusions. The compounds or agents may interfere with transcription, by interacting, for example, with the promoter region. The compounds or agents may interfere with mRNA produced from the fusion (e.g., by RNA interference, antisense technologies, etc.). The compounds or agents may interfere with pathways that are upstream or downstream of the biological activity of the fusion. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present disclosure and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method.

In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, provided herein are screening methods for identifying modulators, i.e., candidate or test compounds or agents (e.g., proteins, peptides, peptidomimetics, peptoids, small molecules or other drugs) which bind to gene fusions of the present disclosure, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly breast cancer.

In one embodiment, the disclosure provides assays for screening candidate or test compounds that are substrates of a cancer marker protein or polypeptide or a biologically active portion thereof. In another embodiment, the disclosure provides assays for screening candidate or test compounds that bind to or modulate the activity of a cancer marker protein or polypeptide or a biologically active portion thereof.

The test compounds of the present disclosure can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a cancer marker mRNA or protein or biologically active portion thereof is contacted with a test compound, and the ability of the test compound to the modulate cancer marker's activity is determined Determining the ability of the test compound to modulate cancer marker activity can be accomplished by monitoring, for example, changes in enzymatic activity, destruction or mRNA, or the like.

### VI. Transgenic Animals

The present disclosure contemplates the generation of transgenic animals comprising an exogenous cancer marker gene (e.g., gene fusion) of the present disclosure or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present disclosure find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present disclosure and are not to be construed as limiting the scope thereof.

### Example 1

### Materials and Methods

### Cell Lines and Specimen Collection

Breast cancer cell lines were purchased from the American Type Culture Collection (ATCC) or obtained from individual collections. Cells were grown in specified media supplemented with fetal bovine serum and antibiotics (Invitrogen), or supplements designated for the media (Lonza). This study was approved by the respective Internal Review Boards and breast cancer samples were obtained from the University of Michigan and the Breakthrough Breast Cancer Research Centre, Institute of Cancer Research (London, UK). Table 2 shows the complete list of cell lines and tissue samples used for this study.

### Paired End Transcriptome Sequencing and Nomination of Gene Fusions

Total RNA was extracted from normal and cancer breast cell lines and breast tumor tissues using Trizol reagent (Invitrogen), and further purified on RNeasy columns (QIAGEN) according to the manufacturer's instructions. Five additional human breast cancer total RNAs were purchased from Origene. The quality of RNA was assessed with the Agilent Bioanalyzer 2100 using RNA Nano reagents (Agilent). Two rounds of polyA selection were performed using SeraMag oligo dT magnetic beads (SeraDyn) following the Illumina protocol. Transcriptome libraries from the mRNA fractions were generated following the RNA-SEQ protocol (Illumina) and size selected using 3% NuSieve agarose gels (Lonza) followed by gel extraction using QIAEX II reagents (QIAGEN) with a gel melting temperature of 32° C. Libraries were quantified using the Bioanalyzer 2100 using the DNA 1000 protocol and reagents (Agilent). Each sample was sequenced in a single lane with the Illumina Genome Analyzer II (40-80 nucleotide read length) or with the Illumina HiSeq 2000 (100 nucleotide read length). Number of reads passing filter for each sample is shown in Table 3. Paired-end transcriptome reads passing filter were mapped to the human reference genome (hg18) and UCSC genes, allowing up to two mismatches, with Illumina ELAND software (Efficient Alignment of Nucleotide Databases). Sequence alignments were subsequently processed to nominate gene fusions using the method described earlier (Maher, C. A. et al. Nature 458, 97-101 (2009); Maher, C. A. et al. Proc Natl Acad Sci USA 106, 12353-8 (2009)). In brief, paired end reads were processed to identify any that either contained or spanned a fusion junction. Encompassing paired reads refer to those in which each read aligns to an independent transcript, thereby encompassing the fusion junction. Spanning mate pairs refer to those in which one sequence read aligns to a gene and its paired-end spans the fusion junction. Both categories undergo a series of filtering steps to remove false positives before being merged together to generate the final chimera nominations.

### Targeted Capture and Sequencing

Following RNA integrity analysis using the Agilent BioAnalyzer 2100 protocol, 74 individual breast carcinomas were placed in two pools. The first pool consisted of 200 ng each of 35 RNAs with RIN values between 3 and 5 and the second pool consisted of 39 RNAs with RIN values between 5.1 and 7.5. The pooled RNAs were depleted of rRNAs using RiboMinus reagents and protocols (Invitrogen). The rRNA depleted pools were converted to paired-end libraries Illumina RNA-SEQ paired end libraries following the standard protocol with the omission of the poly A selection. Following size selection of 250 to 350 bp fragments on agarose gels, the DNA was recovered using the QIAQuick method (QIAGEN) and amplified for 8 cycles using Illumina PE1.0 and PE 2.0 primers and amplification conditions. After purification by the Ampure XP method (Agencourt) the concentration was determined using a Naondrop spectrophotometer. Capture probes were generated for exons 2-10 of MAST1 and MAST2. Primer pairs generating PCR products between 105 and 140 bp were designed and a sequence encoding the T7 RNA polymerase promoter was added to the 5′ end of the forward primer in each pair. The primers are shown in Table 6. 10 cycles of PCR amplification using 10 ng of cDNA plasmids for each gene was performed using HotStar polymerase reagents (QIAGEN). Biotinylated RNA probes were synthesized by in vitro transcription reactions using the T7 Maxiscript protocol (Ambion). Reactions were performed using 0.5 mM ATP, 0.5 mM CTP, 0.5 mM GTP, 0.3 mM UTP, and 0.2 mM biotin-16-UTP. After synthesis at 37° C. for 1 hr, the reactions were digested with DNase I and RNA was purified using the RNAClean method (Agencourt). Each biotinylated RNA probe was adjusted to a concentration of 100 ng/μl and pooled. Pooled probes were hybridized to 2 μg of the previously generated paired-end libraries using conditions and reagents of the SureSelect system (Agilent). Following hybridization for 48 hr, fragments were captured using Dynal M280 streptavidin magnetic beads, washed and eluted using SureSelect protocols. The captured library was reamplified for 14 cycles using Illumina primers and conditions, purified using Ampure XP reagents and submitted for sequencing.

### Array CGH of Breast Cancer Lines

Breast cancer cell line DNAs (ATCC) were labeled and hybridized to Agilent 244K chips using the manufacturer's protocol. Arrays were scanned with an Agilent Microarray Scanner and data were extracted and analyzed with CGH Analytics software.

### Mate Pair Genomic Library Preparation

To detect the genomic rearrangements of NOTCH1 gene in HCC1599 and HCC2218 cells, mate-pair genomic libraries with a 4-4.5 kb insert size were prepared and sequenced. In brief, genomic DNA was isolated from the two cells lines and fragmented by a HydroShear device (Genomic Solutions) to a peak size of 4-5 kb. Mate pair libraries were prepared according to the manufacturer's instructions (Illumina). The libraries were sequenced with the Illumina HiSeq 2000 system.

### Quantitative RT-PCR and Long-Range PCR

To validate the fusion gene transcripts detected by next-generation sequencing, total RNA was isolated from the index cell lines, control cell lines, and breast tissues. Quantitative RT-PCR assays using SYBR Green Master Mix (Applied Biosystems) were carried out with the StepOne Real-Time PCR System (Applied Biosystems). Relative mRNA levels of each chimera shown were normalized to the expression of the housekeeping gene GAPDH. All the oligonucleotide primers were obtained from Integrated DNA Technologies (IDT) and the sequences are listed in Table 6. To detect the genomic fusion junction between NOTCH2 and SEC22B genes in HCC1187 cells, primers were designed flanking the predicted fusion position and PCR reactions were carried out to amplify the fusion fragments. PCR products were purified from agarose gels using the QIAEX II system (QIAGEN) and sequenced by Sanger sequencing methods at the University of Michigan Sequencing Core.

### Immunoblot Detection of MAST2 Fusion Protein and NOTCH1 Protein

For MAST2 fusion protein detection, cell pellets were sonicated in NP40 lysis buffer (50 mM Tris-HCl, 1% NP40, pH 7.4, Sigma), complete protease inhibitor mixture (Roche) and phosphatase inhibitor (EMD bioscience) Immunoblot analysis for MAST2 was carried out using MAST2 antibody from Novus Biologicals. Human β-actin antibody (Sigma) was used as a loading control. For NOTCH1 protein detection, cells were lysed in RIPA buffer containing protease inhibitor cocktail (Pierce). Proteins were separated by SDS-PAGE, transferred to nitrocellulose membranes and probed with antibodies recognizing total NOTCH1 (Cell Signaling), γ-secretase-cleaved NOTCH1 (NICD, Cell Signaling), or beta-actin (Santa Cruz). The signal was detected by chemiluminescence using Immun-Star Western C reagents (Bio-Rad).

Immunoblot analysis for pAKT, total AKT, pERK, total ERK, PTEN were performed after supplement starvation of TERT-HME1 cells for 3 h. Note that, upon supplement starvation pERK could not be resolved as two distinct bands of p42/p44. For the MDAMB-468 cells the cells were treated with fusion specific siRNAs for 2 days and serum starved for 6 hours before probing for the signaling molecules. All the above antibodies were purchased from Cell Signaling. Additional immunoblot screening of signaling molecules was performed at Kinexus, using lysates prepared as previously described.

### Constructs Used for Over-Expression Studies

The ZNF700-MAST1 fusion ORF from BrCa00001 was cloned into pENTR-D-TOPO Entry vector (Invitrogen) following the manufacturer's instructions. Sequence confirmed entry clones in correct orientation were recombined into Gateway pcDNA-DEST40 mammalian expression vector (Invitrogen) by LR Clonase II enzyme reaction. Plasmids with C-terminus V5 tags were generated and tested for protein expression by transfection in HEK293 cells. A full-length expression construct of MAST2 with DDK tag was obtained from Origene.

### Establishment of Stable Pools of TERT-HME1 Cells Expressing MAST and Notch Fusion Alleles

Each of the five MAST fusion alleles, were cloned with an amino terminal FLAG epitope tag into the lentiviral vector pCDH510-B (SABiosciences). Lentivirus was produced by cotransfecting each of the MAST vectors with the ViraPower packaging mix (Invitrogen) into 293T cells using FuGene HD transfection reagent (Roche). Twelve hours posttransfection, the media was changed. Thirty-six hours post-transfection the viral supernatants were harvested, centrifuged at 5000 g for 30 minutes and then filtered through a 0.45 micron Steriflip filter unit (Millipore) TERT-HME1 cells at 30% confluence were infected at an MOI of 20 with the addition of polybrene at 8 μg/ml. Forty-eight hours post-infection, the cells were split and placed into selective media containing 5 μg/ml puromycin. Pools of resistant cells were obtained and analyzed for expression of the MAST fusion constructs by western blot analysis with monoclonal anti-FLAG antibody (Sigma-Aldrich). Stable pools of TERT-HME1 cells expressing the NOTCH fusion alleles, as well as a control NOTCH1 intracellular domain were generated using the same procedures as was done above, with the exception that the NOTCH fusion alleles were cloned into pCDH510B without an amino terminal FLAG epitope tag.

### Cell Transfections

HEK293 cells were transfected with the above mentioned constructs using Fugene 6 reagent (Roche). MAST1 protein over-expression was validated by probing with V5 antibody (Sigma). MAST2 over-expression was validated using DDK antibody (Origene). HMEC-TERT cells were transfected using Fugene 6 and polyclonal populations of cells expressing MAST1, MAST2 or empty vector constructs were selected using geneticin. For siRNA knockdown experiments, Smart-pool siRNAs from Thermo were used (J-004633-06, J-004633-07, and J-004633-08). All siRNA transfections were carried out using oligofectamine reagent (Life Sciences) and three days post transfection the cells were plated for proliferation assays. At the indicated times cell numbers were measured using Coulter Counter. Lentiviral particles expressing the MAST2 shRNA (Sigma, TRCN0000001733) were transduced using polybrene, according to the manufacturer's instructions. Polyclonal populations expressing the MAST2 shRNA sequences were selected using 0.5-1 μg/ml puromycin.

### Colony Formation Assay

Equal number of MDA-MB-468 cells, transduced with scrambled or MAST2 shRNA lentivirus particles were plated and selected using puromycin. After 7-8 days the plates were stained with crystal violet to visualize the number of colonies formed. For quantitation of differential staining, the plates were treated with 10% acetic acid and absorbance was read at 750 nm.

### Confluence Measurements and Wound Healing Assay Using Incucyte

Polyclonal populations of HMEC-TERT over-expressing MAST1, MAST2 or vector control were plated and relative confluence measurements were made at 30 minute intervals using the Incucyte system. Rate of increase in confluence is indicative of increase in cell proliferation. For the wound healing assay, vector control or MAST1 over-expressing cells were plated at high density and 6 hours later, uniform scratch wounds were made using Woundmaker (Incucyte). Relative migration potential of the cells was assessed by confluence measurements at regular time intervals as indicated, over the wound area.

### Chicken Chorioallantoic Membrane Assay

Chicken chorioallantoic membrane (CAM) assay for tumor growth was carried out as follows. Fertilized eggs were incubated in a humidified incubator at 38° C. for 10 days, and then CAM was dropped by drilling two holes: a small hole through the eggshell into the air sac and a second hole near the allantoic vein that penetrates the eggshell membrane but not the CAM. Subsequently, a cutoff wheel (Dremel) was used to cut a 1 cm² window encompassing the second hole near the allantoic vein to expose the underlying CAM. When ready, CAM was gently abraded with a sterile cotton swab to provide access to the mesenchyme and 2×10⁶ cells in 50 μl volume were implanted on top. The windows were subsequently sealed and the eggs returned to the incubator. After 7 days extra-embryonic tumors were isolated and weighed. 5-10 eggs per group were used in each experiment.

### MDA-MB-468-MAST2 Knockdown Xenograft Model

Four week-old female SCID C.B17 mice were procured from a breeding colony at University of Michigan. MDA-MB-468 cells infected with lentivirus constructs of scrambled or MAST2 shRNA were selected for 3 days using puromycin. Mice were anesthetized using a cocktail of xylazine (80 mg/kg IP) and ketamine (10 mg/kg IP) for chemical restraint. MAST2 shRNA or scrambled shRNA knockdown MDA-MB-468 breast cancer cells (4 million) or NOTCH1 fusion allele positive HCC1599 breast cancer cell line (5 million) were resuspended in 100 ul of 1×PBA with 20% Matrigel (BD Biosciences) and implanted into right and left abdominal-inguinal mammary fat. Ten mice were included in each group. Two weeks after tumor implantation, HCC1599 xenograted mice were treated with γ-secretase inhibitor (DAPT) dissolved in 5% ethanol in corn oil (IP). Mice in control group also received 5% ethanol in corn oil as vehicle control. Tumor growth was recorded weekly by using digital calipers and tumor volumes were calculated using the formula (R/6) (L×W2), where L=length of tumor and W=width.

### Inhibition of Notch and Cell Proliferation Assay

For cell proliferation assays, cells were seeded into 96-well plates in triplicate and allowed to attach overnight before drug treatment. The γ-secretase inhibitor DAPT (EMD Biosciences) was added to the cultures the next day at concentrations of 0, 0.3, 1, and 3 μM. Relative cell numbers were measured by WST-1 assays at indicated time points following the manufacturer's instructions (Roche).

### Luciferase Assay

Breast cancer cells were seeded into 24-well dishes in triplicate and allowed to attach overnight. Cells were then infected with a Notch-reporter construct Lenti-RBPJ-firefly luciferase together with a Lenti-CMV-Renilla luciferase control (SABiosciences/QIAGEN). The two lentiviral stocks were mixed at a ratio of 50 Notch reporters to 1 CMV control and a single mixture was used to infect all recipient cell lines at a MOI of 100. Following incubation for 48 hours, cell lysates were prepared and measured for Notch activity using Promega Dual Luciferase reagents and Passive Lysis Buffer. Firefly luciferase levels were normalized using corresponding Renilla luciferase levels for each cell line. To confirm that Notch pathways are activated in the index cell lines through Notch gene rearrangements, the activated NOTCH1 and NOTCH2 alleles were cloned from HCC1599, HCC2218, and HCC1187 into a pcDNA3.1 vector. These expression constructs, pcDNA3.1-1599-NOTCH1, pcDNA3.1-2218-NOTCH1, and pcDNA3.1-1187-NOTCH2 and positive control NOTCH1-NICD, were individually transfected into 293T cells along with the pGL4-RBPJ-4X reporter plasmid and pTKRenilla luciferase control plasmid. Cells were harvested for luciferase activity assays 24 hours after transfection and assayed as above.

### Results

### Transcriptome Sequencing of Breast Carcinoma

A panel of 41 breast cancer cell lines, and 37 breast cancer tissues, along with 8 benign breast epithelial cell lines and 2 benign breast tissues, was sequenced by paired-end sequencing of transcriptome libraries followed by analysis for gene fusions using a previously developed chimera discovery pipeline (Maher, C. A. et al., Nature 458, 97-101 (2009); Maher, C. A. et al. Proc Natl Acad Sci USA 106, 12353-8 (2009)). 42 of the samples were ER (estrogen receptor) positive, 21 exhibited amplified ERBB2, and 26 were classified as triple negative (Tables 2 and 3). Fusion transcript discovery and validation lead to the identification of 372 gene fusions, at an average of over four gene fusions per breast cancer sample (Table 4). Gene fusions were identified in all 41 breast cancer cell lines and all but 3 primary tumors. A slightly higher number of gene fusions was detected in the cell lines compared to primary tumors.

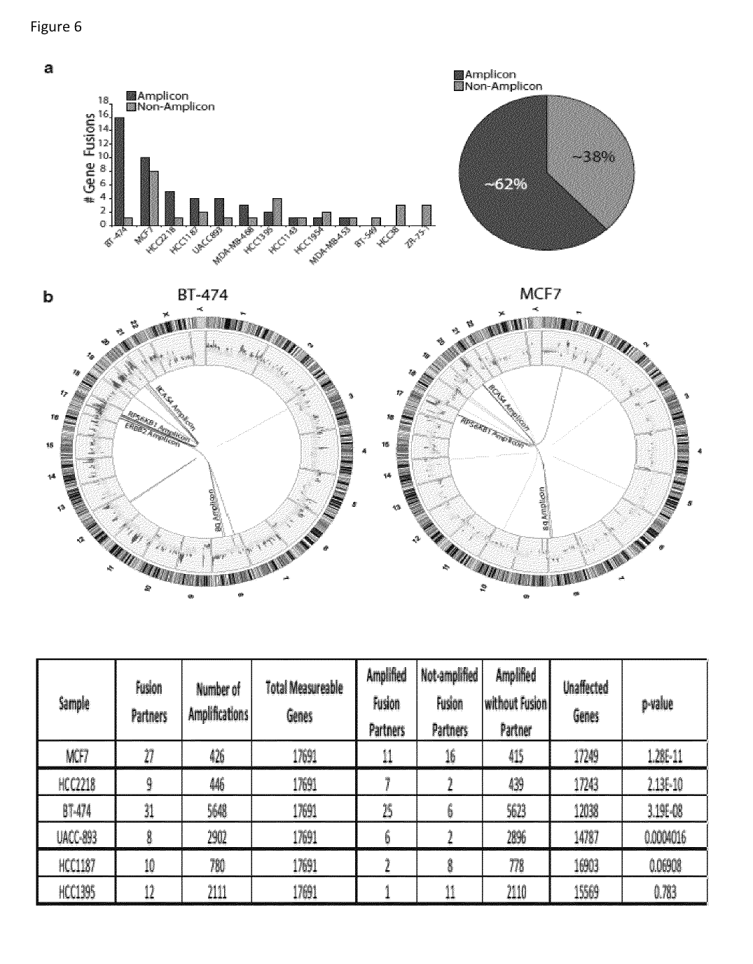

A closer examination of the chromosomal coordinates of the fusion partner genes revealed that a majority of the gene fusions clustered in regions of chromosomal amplifications (FIG. 6). To study this further, a set of 6 breast cell lines with matched RNA-Seq and array CGH data was analyzed (FIG. 6). For each sample, the probe log-ratio values overlapping each gene were averaged and a threshold of >2× copy number was applied to call amplifications. Using a one-sided Fisher exact test statistically significant associations between fusion gene partners and regions of amplification in 6 independent samples were observed (FIG. 6b).

Chromosome 17 harbors the ERBB2 amplicon and an adjacent amplicon that includes genes such as BCAS3, RPS6 KB1, and TMEM49 among others, accounted for a third of all the gene fusions in samples with CGH data. (Table 4). Other recurrent loci harboring multiple gene fusions include the BCAS4 amplicon on chr20 and the chr8q amplicon. No single gene fusion from the more than 350 identified here was found to be recurrent in the compendium, even as several fusion genes did appear in combination with different fusion partners. For example, three fusions each involving IKZF3 and BCAS3 as 3′ partners were found in three different cell lines—all with different 5′ partners; likewise TRIM37 was a common 5′ partner in three distinct gene fusions with different 3′ partners. Overall, 24 genes were found to be recurrent fusion partners, often associated with amplicons (Table 4).

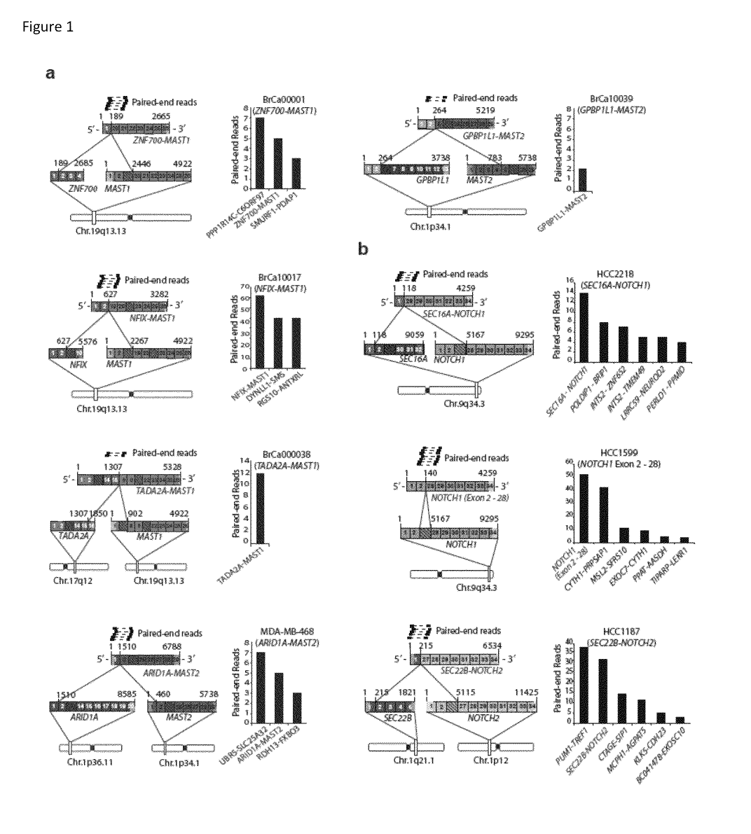

In order to focus on potentially tumorigenic ‘driver’ fusions, the gene fusions were prioritized based on the known cancer-associated functions of component genes such as if the 3′ partner was a kinase, oncogene, tumor suppressor or known to be fusion partners in the Mitelman Database of chromosomal aberrations in cancer. In the sample set, 5 cases of fusions of MAST family kinases and 7 cases with fusions of genes in the Notch family were identified. Singleton fusions with open reading frames that could potentially be considered ‘drivers’ included SPRED1-BUB1B (kinase), MYO15B-MAP3K3 (kinase), BCL2L14-ETV6 (ETS transcription factor), MSI2-NEK8 (kinase), and SEC11C-MALT1 (oncogene) among others (Tables 1 and 5). Notch and MAST kinase fusions were mutually exclusive and occurred mostly in ER negative breast carcinoma samples (Table 1 and FIG. 1).

### MAST Gene Fusions in Breast Carcinoma

Three independent cases of MAST gene fusions were identified by initial transcriptome sequence analyses-ZNF700-MAST1 in breast cancer tissue BrCa00001, NFIX-MAST1 in breast carcinoma BrCa10017, and ARID1A-MAST2 in a triple negative (ER-/PR-/ERBB2-) breast cancer cell line MDA-MB-468 (FIG. 1a). These gene fusions were among the top scoring fusions observed in their respective index samples, based on the number of unique paired end reads supporting the chimeric transcripts. These index samples ranked among the highest levels of expression of MAST1 (in BrCa00001 and BrCa10017) and MAST2 (in MDA-MB-468) in the compendium of more than 350 cancer samples encompassing more than 17 different tissue types. FISH-based screening was not feasible for genes that are in close proximity (e.g., ZNF700, NFIX, and MAST1 are less then 1 Mb apart on Chr 19) or regions of highly repetitive genomic sequences. As high throughput next generation sequencing now enables the detection of genetic aberrations at a resolution far superior to cytogenetic and FISH based approaches, a targeted sequencing approach was used to screen additional samples for MAST gene fusions. A transcriptome library of 92 pooled breast carcinoma RNAs was generated and captured in solution with biotinylated baits encompassing the 5′ exons 2-10 of MAST1 and MAST2. The captured library was sequenced and analyzed as before. Two new MAST gene fusions were discovered using this strategy. TADA2AMAST1 and GPBP1L1-MAST2. The samples harboring MAST gene fusions are distinct from those with Notch family gene fusions.

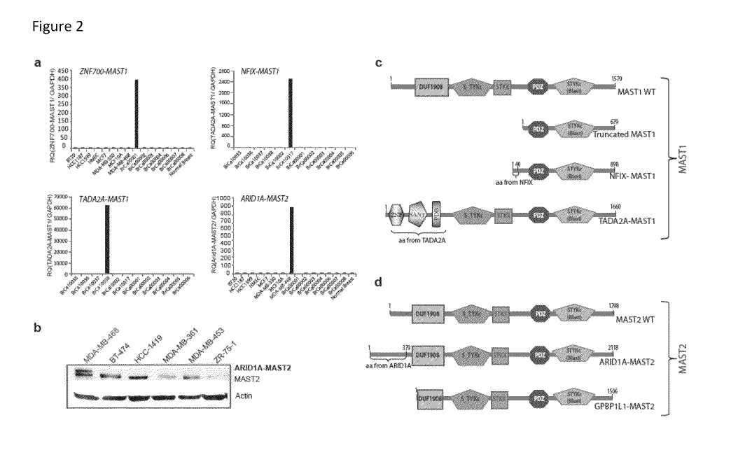

Each of the fusions was confirmed by fusion-specific PCR in the respective samples (FIG. 2a). As a working antibody was available for MAST2, the expression of the fusion protein from the ARID1A-MAST2 gene fusion was validated in the breast cancer cell line MDA-MB-468 (FIG. 2b). All five MAST fusions encoded contiguous open reading frames, retaining the serine/threonine kinase and PDZ domains of 3′ MAST genes (FIG. 2c,d). The predicted open reading frames of the MAST fusions identified each retain intact PDZ and serine/threonine kinase domains. Thus overall, five novel gene fusions encoding MAST1 and MAST2 in a cohort of a little over 100 breast cancer samples and more than 40 cell lines were identified, indicating that the novel serine/threonine kinase family gene fusions represent a subset of up to 5% of breast cancers. As these are kinase fusions, they also provide therapeutic targets.

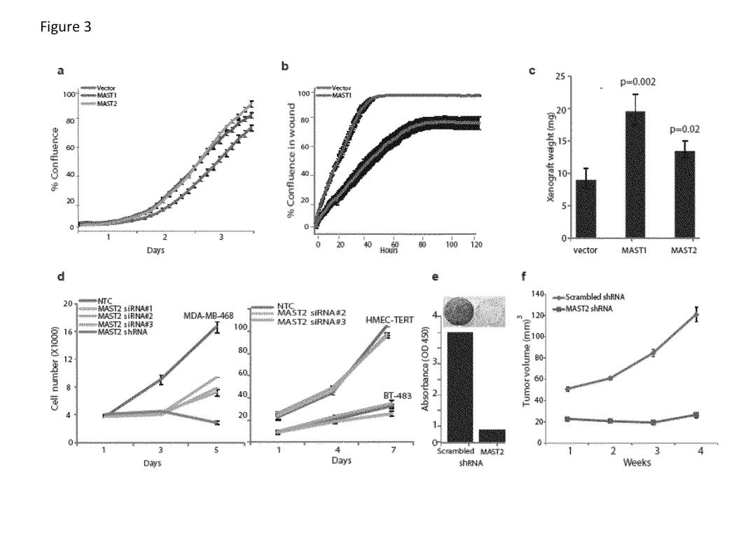

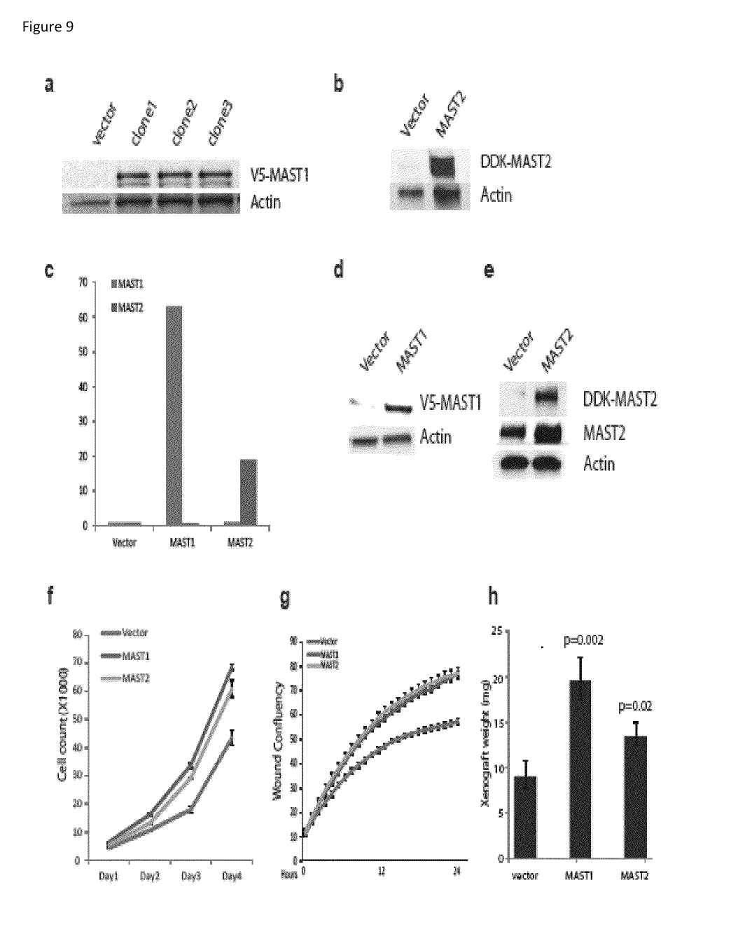

Next, the functional aspects of MAST fusion proteins were investigated. The ZNF700-MAST1 fusion transcript encodes a truncated MAST1 protein that retains the kinase (as well as PDZ) domain. The fusion encoded open reading frame from the index sample, breast cancer tissue BrCa00001, was cloned into an expression vector. A commercially available full-length MAST2 expression construct was used to mimic the function of ARID1A-MAST2 over-expression, as this fusion encodes nearly full length MAST2 (along with a 379 amino acid segment from ARID1A). To assess the potential oncogenic functions of MAST genes, epitope tagged truncated MAST1 and full length MAST2 were ectopically over-expressed in the benign breast cell line, HMEC-TERT. Expression of the respective constructs was confirmed using anti-V5 and anti-DDK antibodies (FIG. 9a, b). Next, polyclonal populations of HMEC-TERT cells overexpressing MAST1 and MAST2 were generated (FIG. 9c, d). Using the Incucyte system to measure cell proliferation in real time, both the MAST1 and MAST2 overexpressing cells showed a growth advantage over vector control cells in confluence measurements (FIG. 3a). MAST1 and MAST2 over-expressing HMEC-TERT cells also showed increased migration potential in a wound healing assay (FIG. 3b). Furthermore, MAST1 and MAST2 over-expressing HMEC-TERT cells showed a significantly increased growth in a chicken chorioallantoic membrane (CAM) assay, as compared to control cells (FIG. 3c) and a wound healing assay. Overall, these findings indicate that fusion encoded truncated MAST1 and full length MAST2 over-expression can impart growth and proliferative advantage thereby promoting an oncogenic phenotype.

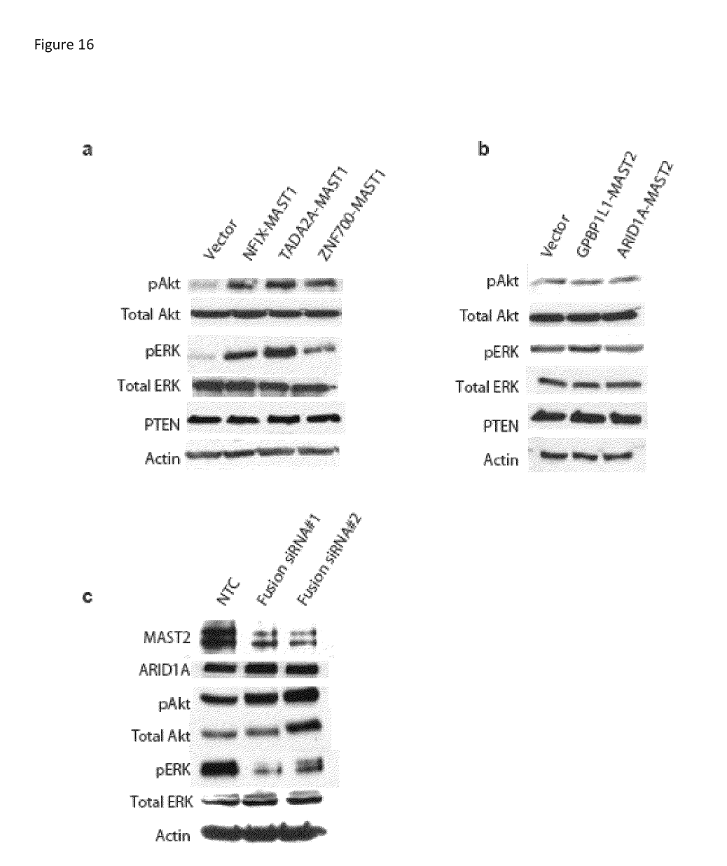

With the identification of the newer MAST fusions using the pooled transcript capture and sequencing approach and for a more comprehensive analysis of all the MAST fusions identified in the study, MAST1/MAST2 fusions were cloned and expressed in a lentiviral expression system. Consistent with the earlier observations, TERT-HME1 cells overexpressing the five MAST fusions (FIG. 3a) also displayed higher rates of cell proliferation compared to FLAG vector control cells (FIG. 3b). Overall, these results indicate that ectopic expression of the MAST fusions impart growth and proliferative advantage in benign breast epithelial cells. To identify pathways that could be activated by the MAST fusions to confer the growth advantage phenotype observed, more than 20 different signaling molecules involved in more than 10 different pathways were interogated. Both services from Kinexus Bioinformatics Corp. and an in house immunoblot analysis (with antibodies from Cell Signaling) were employed for this purpose (Table 8 and FIG. 16). Of the pathways tested, levels of phosho AKT (pAKT) and phospho ERK1/2 (pERK) displayed differential levels. As shown in FIG. 16a, ectopic expression of MAST1 fusions activated both the pAKT and pERK signaling pathways. Overexpression of MAST2 fusions did not lead to activation of AKT/ERK pathways (FIG. 16b). These data implicate MAST proteins as key modulators of cell proliferation resulting in an oncogenic phenotype seen in fusion positive cells.

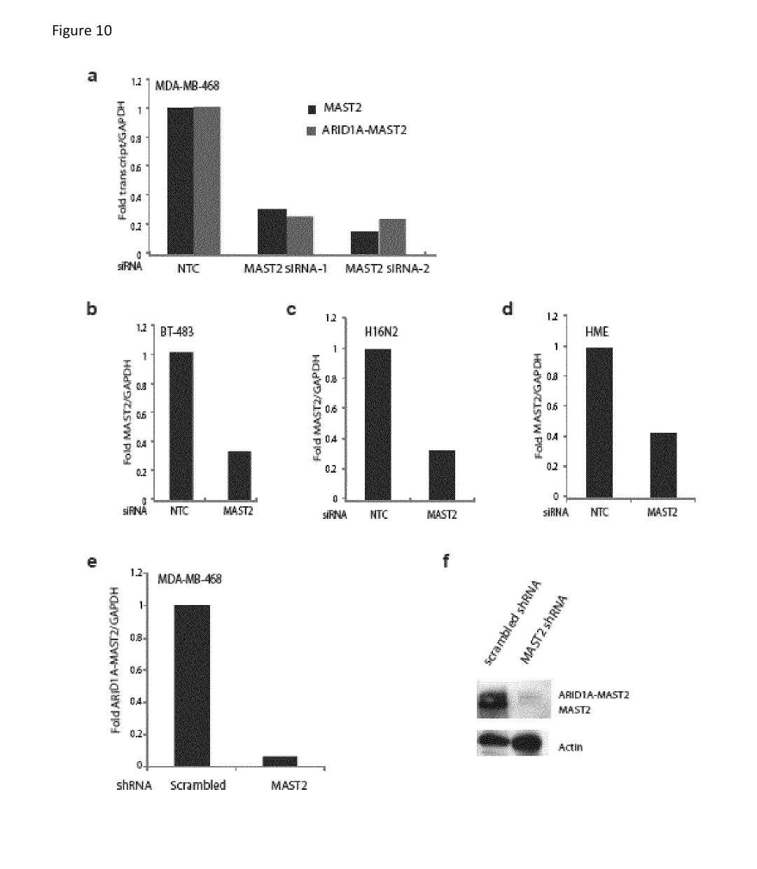

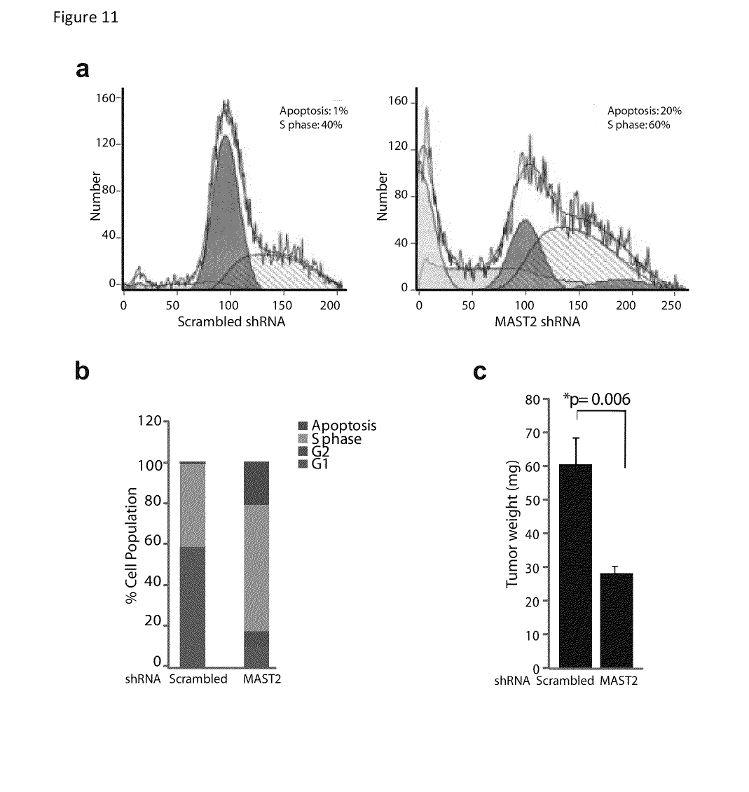

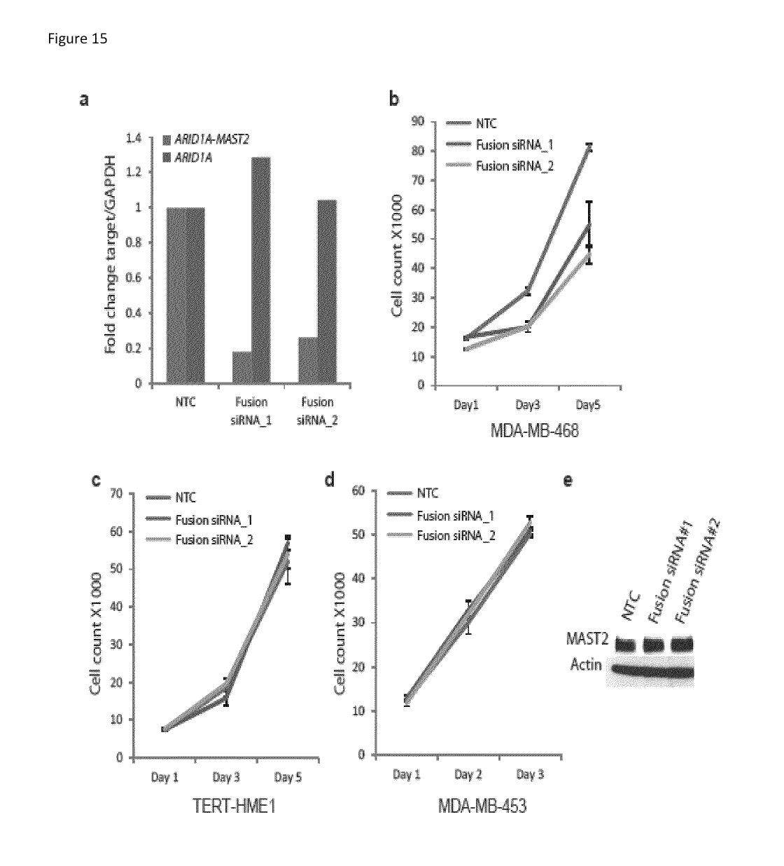

To study the role of the endogenous ARID1A-MAST2 fusion in MDA-MB-468 cells, multiple independent MAST2 siRNAs were used to achieve a marked knockdown of the MAST2 fusion (FIG. 10a). These siRNAs showed significant growth inhibitory effects in cell proliferation assays in MDA-MB-468 cells (FIG. 3d, left panel). Knockdown of MAST2 in fusion negative benign breast cells, HMEC-TERT and a breast cancer cell line BT-483 did not have an effect on cell proliferation (FIG. 3d right panel), although a significant reduction in the levels of the wild-type MAST2 transcript was achieved (FIG. 11b-d). The fusion-specific siRNAs also did not alter the levels of either the ARID1A transcript (FIG. 15a) or protein (FIG. 16c). Together this indicates that in MDA-MB-468 cells the specific knockdown of the ARID1A-MAST2 fusion alone is sufficient to reduce cell proliferation. Next, MDA-MB-468 cells treated with fusion-specific siRNAs were assessed for levels of pAKT and pERK. Shown in FIG. 16c, knockdown of the ARID1AMAST2 fusion results in decreased levels of pERK.

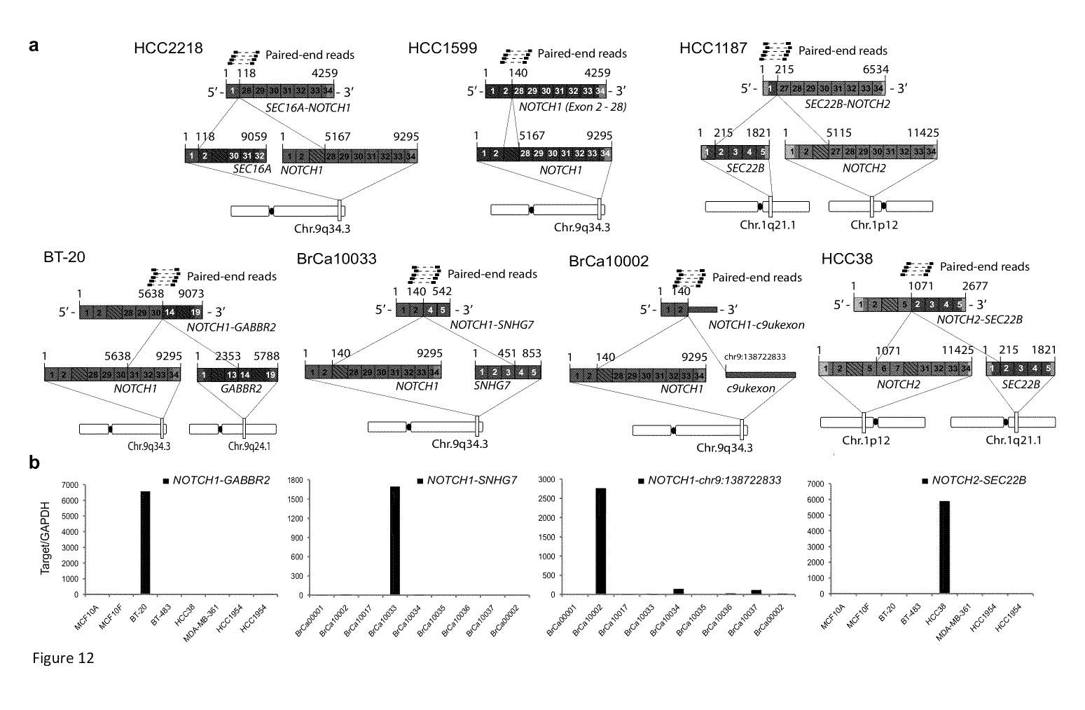

To characterize the effects of the ARID1A-MAST2 fusion in MDA-MB-468 cells further, shRNA targeting MAST2, which displayed efficient knockdown of ARID1A-MAST2 fusion at both the transcript (FIG. 11e) and protein level (FIG. 11f) was used. MDA-MB-468 cells treated with MAST2 shRNA exhibited a dramatic reduction in growth as demonstrated in a colony formation assay (FIG. 3e), as well as showed increased apoptosis with S-phase arrest (FIG. 12a, b). MAST2 shRNA treated MDA-MB-468 cells did not survive long-term culturing, therefore, in vivo experiments were carried out using MDA-MB-468 cells transiently transfected with MAST2 shRNA. A reduction in tumor burden in the chicken chorioallantoic membrane assay was observed (FIG. 13c). In the mouse xenograft model, MDA-MB-468 cells transiently transfected with MAST2-shRNA, but not the scrambled control, failed to establish palpable tumors over a time course of 4 weeks (FIG. 31). Taken together, the knockdown studies show that the ARID1A-MAST2 fusion is a critical driver fusion in MDA-MB-468 cells.

### Notch Gene Fusions in Breast Carcinoma

Fusion transcript discovery and validation detected a high frequency of Notch gene rearrangement with 7 rearrangements involving either NOTCH1 or NOTCH2 in the samples tested (Table 1, FIG. 1b, and FIG. 12).

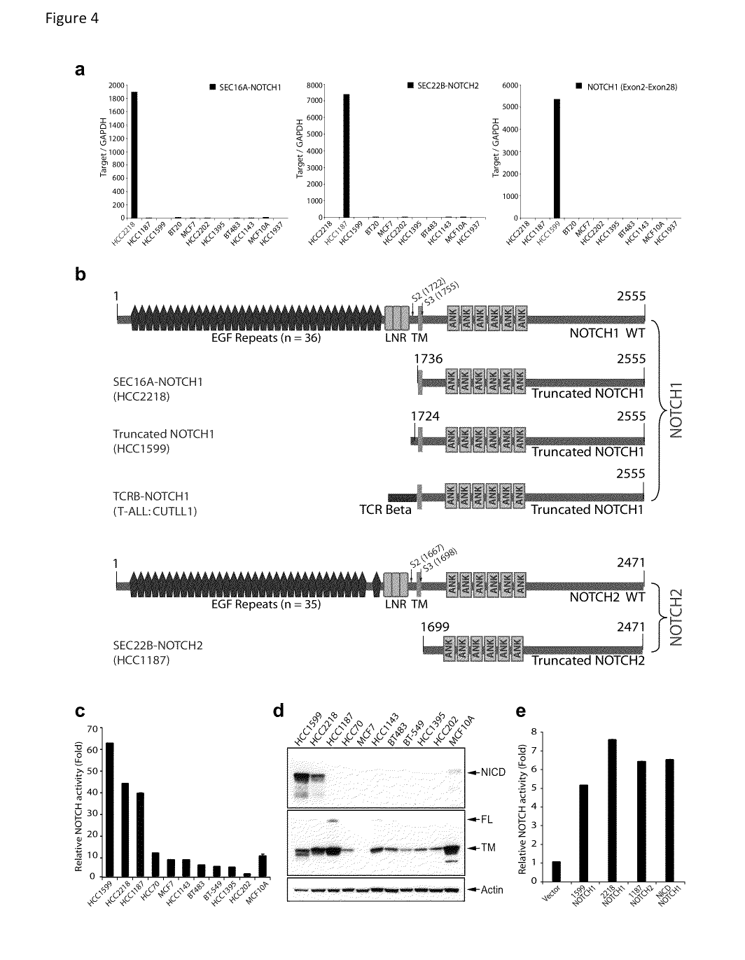

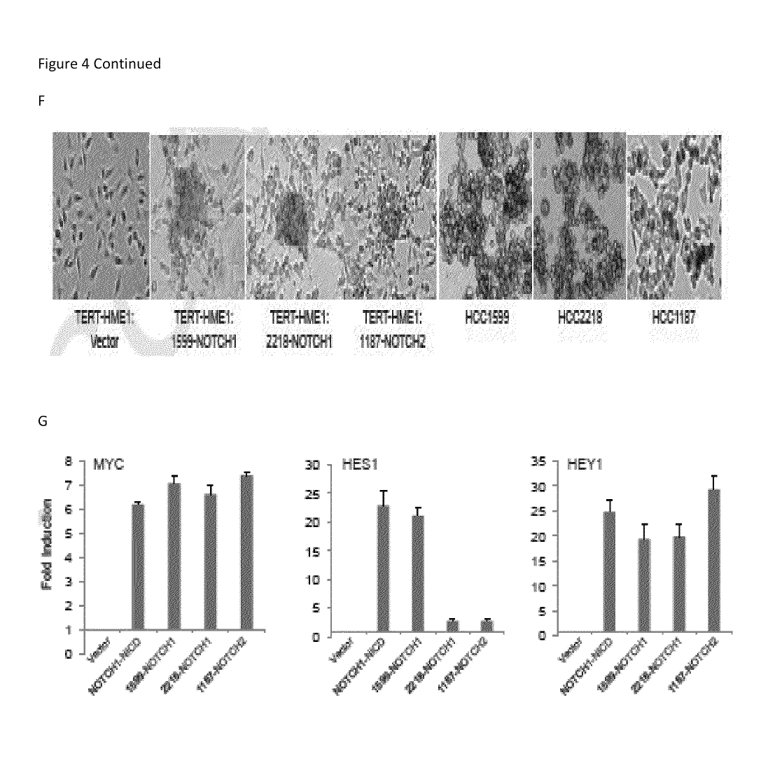

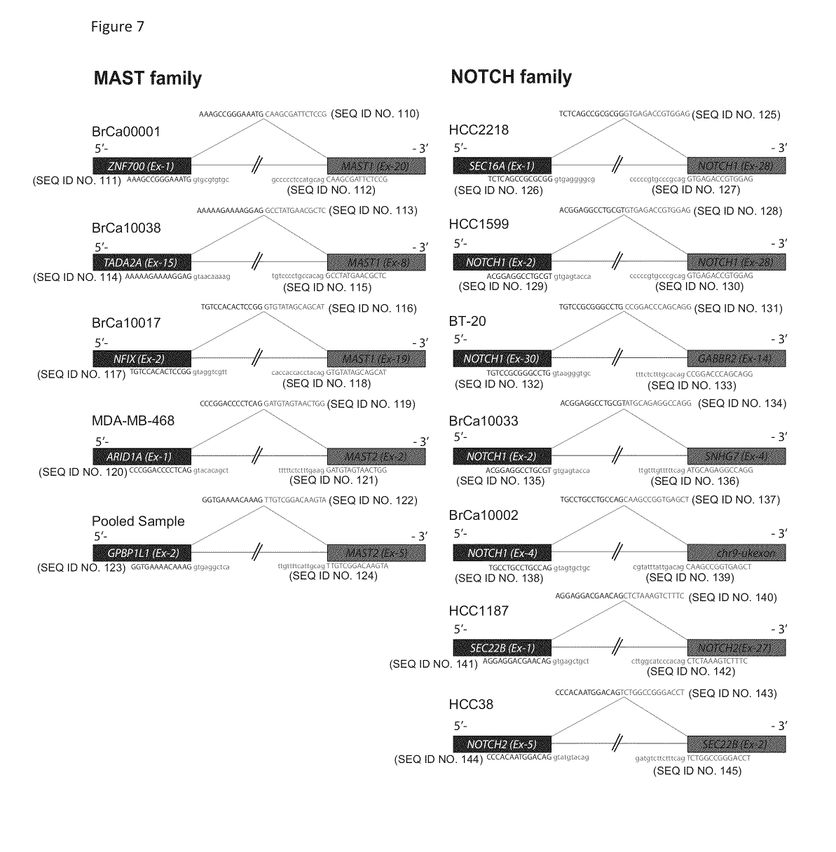

All of the Notch family gene rearrangements were found in ER negative breast carcinomas, and all but one in triple negative breast carcinomas. While both 5′ and 3′ fusion transcripts of Notch were identified in breast cancer samples (FIGS. 7, 12), three ER negative breast cancer cell lines that expressed the 3′ NOTCH1 or NOTCH2 fusion transcripts were used for functional studies (FIG. 4a,b). The HCC2218 cell line expresses a chimeric transcript derived from exon 1 of SEC/6A and exons 28-34 of the nearby NOTCH1 gene. The HCC1187 cell line expresses a chimeric transcript containing exon 1 of SEC22B fused to exons 27-34 of NOTCH2. Finally, the HCC1599 cell line expresses a NOTCH1 intragenic in-frame fusion transcript with exon 2 spliced to exon 28. The fusion transcripts in the 3 breast cancer lines retain the exons encoding the NICD, responsible for inducing the transcriptional program following Notch activation.

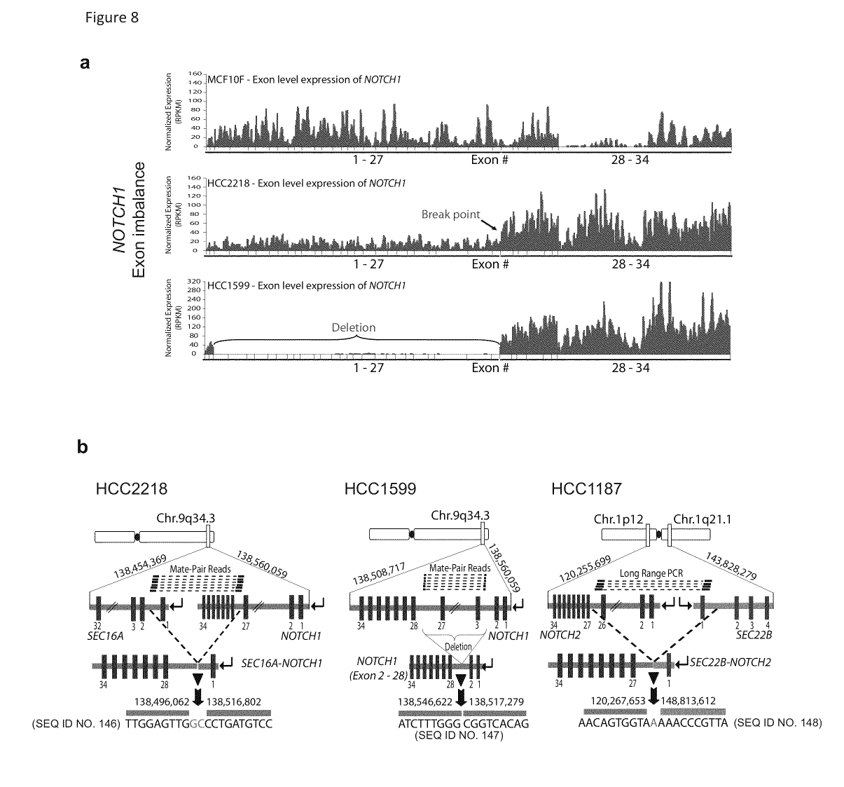

To determine whether the observed fusions transcripts were the result of DNA rearrangements, mate-pair genomic library sequencing and long-range genomic PCR was performed to identify DNA breakpoints associated with the gene loci involved in the fusion transcripts (FIG. 8b). A fusion fragment from genomic DNA was PCR amplified and sequenced using primers based on chimeric mate pair fragments for both the HCC2218 and HCC1599 cell lines. The HCC1187 genome was analyzed directly by long-range PCR using primers in regions predicted to flank the fusion breakpoint. All three samples contained DNA rearrangements directly responsible for the generation of the observed fusion transcripts. In HCC2218 genomic DNA, a junction is present between intron 1 of SEC16A and intron 27 of NOTCH1. In HCC1187 genomic DNA, a junction is present between intron 1 of SEC22B and intron 26 of NOTCH2. Finally, in HCC1599, a deletion is detected between introns 2 and 27 of NOTCH1. Thus, all three breast cancer lines contain genetic aberrations producing fusion transcripts encoding 5′ truncated members of the Notch family.

The Notch fusion transcripts are abundantly expressed and are specific to samples harboring DNA rearrangements. SYBR Green QPCR experiments using primers on either side of each of the transcript fusion junctions detected expression exclusively in the sample harboring the underlying DNA rearrangements (FIG. 4a, and FIG. 12b). RNA-SEQ expression maps of NOTCH1 further support both the type of rearrangement and high level of expression of the fusion transcripts (FIG. 8a). The top panel of FIG. 8a displays the expression across all exons of the wild-type NOTCH1 allele in the normal breast line MCF10F. In contrast, the expression map for NOTCH1 in HCC2218 cells expressing the SEC16A-NOTCH1 fusion exhibits a dramatically increased coverage of the exons, 28-34, contained in the fusion transcript (FIG. 8a, middle panel). Additionally, in HCC1599, there is a complete absence of RNA-SEQ coverage for exons 3-27 of NOTCH1 (FIG. 3a, lower panel), supporting a homozygous or hemizygous intragenic deletion generating the aberrant NOTCH1 transcript, consistent with the genomic DNA sequencing results shown earlier.

The predicted open reading frames for the NOTCH1 and NOTCH2 fusion transcripts are illustrated in FIG. 4b along with wild type NOTCH1 and NOTCH2 reading frames. The two activating cleavage sites S2 and S3 are also shown for NOTCH1 and NOTCH2. For both the SEC16A-NOTCH1 fusion and the intragenic HCC1599 NOTCH1 fusion, the predicted ORFs initiate after the S2 cleavage site, but before the S3 cleavage site. The encoded proteins would be predicted to mimic the S2 cleavage product produced during Notch activation and require cleavage at the S3 site by γ-secretase to release NICD. These fusions bear a great deal of similarity to the TCRB-NOTCH1 fusion in the T cell adult lymphocytic leukemia line CUTLL1 30, which requires cleavage by γ-secretase for activity. In contrast, the SEC22B-NOTCH2 fusion ORF is predicted to initiate just after the γ-secretase S3 cleavage site. The resultant protein would be nearly identical to the engineered NICD constructs used by many investigators studying the Notch pathway. It would be predicted to be highly active and to not require cleavage by γ-secretase for its activity (FIG. 4b).

It was next evaluated whether the Notch fusion alleles identified above were capable of activating the Notch pathway in the index cases and when introduced into recipient cells. The activity of the Notch pathway in a panel of breast cell lines was measured using a dual luciferase assay following lentiviral delivery of Notch reporter and control vectors into recipient cells. The results presented in FIG. 4c demonstrate substantially higher Notch responsive transcriptional activity in the three cell lines containing Notch fusions, compared with other breast cell lines tested. This indicates that each of the three Notch fusions, expressed at its endogenous level, is capable of activating the expression of Notch responsive genes in the carcinoma cells containing the fusion. Further evidence supporting an activated Notch pathway is obtained from Western blot analysis of breast carcinoma lines, presented in FIG. 4d. Using an antibody specific to the γ-secretase cleaved active form of the NOTCH1-NICD, both HCC1599 and HCC2218 exhibit high levels of NICD, consistent with the fusion protein acting as a substrate for activation by γ-secretase. MCF10A cells do contain a substantially lower level of NICD, consistent with previous reports, while other breast carcinoma lines exhibit very little activated NOTCH1 NICD. It should be noted that HCC1187, which contains a NOTCH2 fusion gene, exhibits little detectable NOTCH1-NICD. Most breast cancer lines express NOTCH1, as detected with an antibody recognizing the intact NOTCH1 transmembrane protein (FIG. 4d, middle panel). However, only the two cell lines with NOTCH1 fusions alleles show high levels of activated NICD. To further demonstrate the high Notch signaling activity was a result of the rearranged Notch alleles in the three index cell lines, ectopic expression of the fusion alleles was tested. Expression vectors encoding the ORFs from each of the three fusion alleles were co-transfected with a Notch reporter plasmid and a Renilla control vector into HEK293T cells. An expression vector encoding the NICD of NOTCH1 was included as a positive control. The normalized Notch activities as shown in FIG. 4e demonstrate that the three fusion alleles have the capacity to elicit Notch responsive transcription at levels equivalent to NICD itself.

The three index breast cell lines containing the Notch fusions (HCC1599, HCC2218, and HCC1187) exhibit decreased cell-matrix adhesion and grow in suspension, or as weakly adherent clusters, unlike the majority of breast carcinoma cell lines (FIG. 4f). Additionally, a recent study on the effects of expressing NOTCH1-NICD in the benign mammary epithelial line MCF10A demonstrated a loss of cell-matrix adhesion and the tendency to form clusters. The effects of expressing the NOTCH fusions in the immortalized mammary epithelial cells TERT-HME1 was assayed. The NOTCH1 fusion alleles from HCC1599 and HCC2218, and the NOTCH2 fusion allele from HCC1187 were cloned into a lentiviral expression vector. Following lentiviral transduction, stable pools of TERT-HME1 cells expressing the fusion alleles were established using puromycin selection. Striking morphological changes are seen in the stable pools expressing the Notch fusion alleles (FIG. 4f), consistent with those previously reported in NOTCH1-NICD expressing MCF10A ells 25. The parental and vector transduced TERT-HME1 cells exhibit adherent epithelial properties, while the Notch fusion expressing cells lose adherence and propagate as weakly attached clusters, similar to the morphology of the index lines harboring the Notch fusion alleles. Furthermore, the expressed fusion alleles dramatically induced expression of the well characterized Notch target genes, MYC, and two members of the hairy/enhancer of split family of transcription factors, HES1 and HEY1 (FIG. 4g).

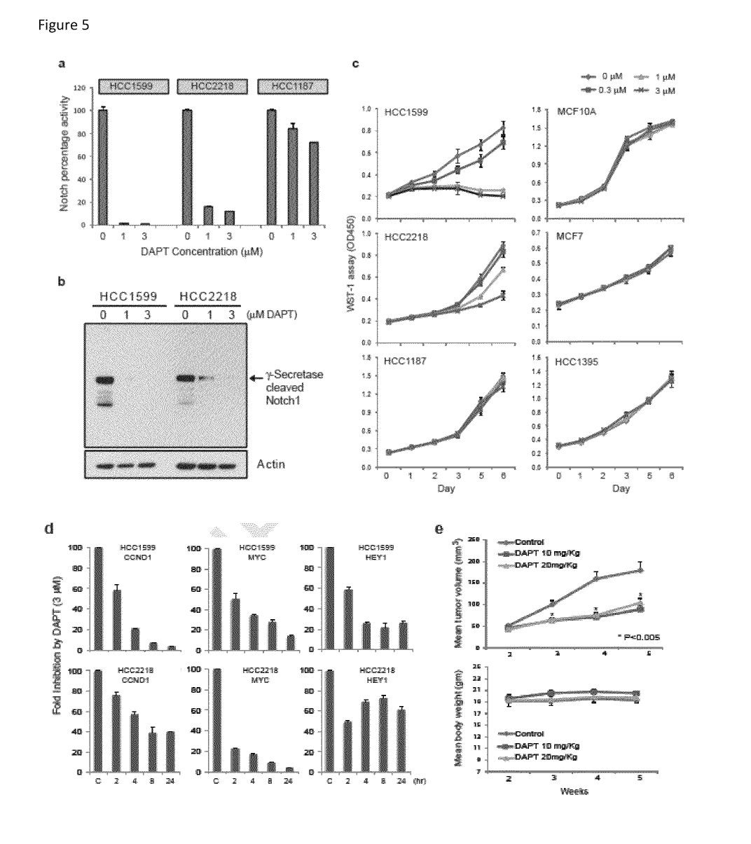

Notch fusion alleles provide a target for therapeutic intervention. The three characterized Notch fusions represent two functional classes. The first class, exemplified by the HCC2218 and HCC1599 fusions, produces a protein similar to that produced by the ADAM17/TACE catalyzed S2 cleavage, which occurs during ligand activation of the Notch pathway. The second class, exemplified by the HCC1187 fusion, produces a protein similar to the NICD produced after cleavage at S3 by γ-secretase. The first class requires cleavage at S3 site by γ-secretase to release NICD, and thus would be expected to be sensitive to γ-secretase inhibitors (GSIs). The second class would be unaffected by GSIs, as the fusion generates an ORF similar to NICD. To test this, stable Notch reporter cell lines were established from each of the three Notch fusion positive carcinoma lines by infection with a lentivirus carrying the Notch responsive promoter driving firefly luciferase. Each of the three cell lines was treated with the γ-secretase inhibitor DAPT 31, and luciferase activity was measured in cell lysates 24 hours later. FIG. 5a shows a dramatic reduction of Notch reporter activity upon DAPT treatment in HCC1599 and HCC2218, which express fusion proteins requiring γ-secretase cleavage for activation. On the other hand, Notch reporter activity is only slightly diminished by DAPT in HCC1187, which expresses a γ-secretase independent Notch fusion allele. Western blot analyses of NICD levels in HCC1599 and HCC2218 following DAPT treatment, are shown in FIG. 5b. DAPT treatment dramatically reduced NICD levels in both cell lines, with nearly complete elimination in HCC1599. These results precisely mirror those obtained in the luciferase assay shown in FIG. 5a, with HCC1599 cells showing slightly greater sensitivity to DAPT than HCC2218 cells. Furthermore, index cell lines exhibit dependence on Notch signaling for proliferation and survival. Effects of the γ-secretase inhibitor DAPT on the proliferation of a panel of breast cell lines are shown in FIG. 5c. A panel of six breast cell lines were treated with DAPT at 0, 0.3, 1, and 3 μM, and cell proliferation was measured using a WST-1 assay over a six day time course. The HCC1599 cell line, with a GSI sensitive NOTCH1 fusion, exhibited a dramatic reduction in proliferation with all concentrations of the inhibitor. HCC2218 also expresses a GSI sensitive NOTCH1 fusion and exhibits significant reduction in proliferation following DAPT treatment. HCC1187, which expresses a GSI independent NOTCH2 fusion, shows no reduction in proliferation upon DAPT treatment, as do the other breast cell lines not expressing Notch fusion alleles.

Treatment with the γ-secretase inhibitor DAPT repressed Notch target gene expression in a rapid manner. Expression levels of the Notch target genes CCND1, MYC, and HEY1 were monitored over a 24-hour treatment time course in the cell lines harboring Notch fusions dependent on γ-secretase processing (FIG. 5d). The reduction in MYC and CCND1, two genes previously identified to play a key role in mouse mammary tumorigenesis induced by, further support the possibility that GSIs may be useful in treating cancers harboring activated Notch alleles. This was tested further by establishing a xenograft tumor model of HCC1599 in immunodeficient mice. Treatment with DAPT significantly reduced tumor volume compared with untreated controls (FIG. 5e). No effect on overall body weight was observed with the doses of DAPT used.

TABLE 1

| | | | | | | |
|---|---|---|---|---|---|---|
| Sample | Type | Fusion detected | Read # | ER | PR | ERBB2 |

MAST family

| | | | | | | |
|---|---|---|---|---|---|---|
| BrCa00001 | Tumor | ZNF700-MAST1 | 5 | pos | neg | neg |
| BrCa10017 | Tumor | NFIX-MAST1 | 65 | neg | neg | pos |
| BrCa10038 | Tumor | TADA2A-MAST1 | 12 | neg | neg | pos |
| MDA-MB-468 | Cell line | ARID1A-MAST2 | 5 | neg | neg | neg |
| BrCa10039 | Tumor | GPBP1L1-MAST2 | 2 | pos | neg | neg |

Notch family

| | | | | | | |
|---|---|---|---|---|---|---|
| HCC2218 | Cell line | SEC16A-NOTCH1 | 14 | neg | neg | pos |
| HCC1599 | Cell line | NOTCH1 internal deletion | 53 | neg | neg | neg |
| BT-20 | Cell line | NOTCH1-GABBR2 | 21 | neg | neg | neg |
| BrCa10002 | Tumor | NOTCH1-chr9: 138722683 | 14 | neg | neg | neg |
| BrCa10033 | Tumor | NOTCH1-SNHG7 | 5 | neg | neg | neg |
| HCC1187 | Cell line | SEC22B-NOTCH2 | 30 | neg | neg | neg |
| HCC38 | Cell line | NOTCH2-SEC22B | 6 | neg | neg | neg |

Singleton fusions

| | | | | | | |
|---|---|---|---|---|---|---|
| MDA-MB-453 | Cell line | MYO15B-MAP3K3 | 4 | neg | neg | pos |
| BrCa10026 | Tumor | MSI2-NEK8 | 30 | pos | neg | pos |
| HCC38 | Cell line | SPRED1-BUB1B | 10 | neg | neg | neg |
| BrCa00006 | Tumor | STK3-RIMS2 | 5 | pos | neg | neg |
| HCC1954 | Cell line | INTS1-PRKAR1B | 22 | neg | neg | pos |
| HCC1569 | Cell line | PTPRJ-LPXN | 53 | neg | neg | pos |
| BrCa10025 | Tumor | BCL2L14-ETV6 | 5 | neg | neg | neg |
| BrCa10035 | Tumor | RELB-CBLC | 13 | pos | neg | pos |
| ZR-75-1 | Cell line | FOXJ3-CAMTA1 | 10 | pos | pos | neg |
| HCC1419 | Cell line | VAV2-TRUB2 | 18 | pos | neg | pos |
| BrCa10001 | Tumor | SEC11C-MALT1 | 20 | pos | pos | neg |
| SUM190PT | Cell line | KLH22-CRKL | 9 | neg | neg | pos |
| BrCa10021 | Tumor | NPAS3-MIPOL1 | 10 | pos | pos | neg |
| BrCa10025 | Tumor | RFX3-RB1 | 6 | neg | neg | neg |
| BrCa10037 | Tumor | KDM4A-RASSF5 | 107 | neg | neg | neg |
| BrCa10014 | Tumor | MACROD1-VEGFB | 101 | pos | pos | neg |
| BrCa10006 | Tumor | GPATCH8-BRIP1 | 16 | pos | pos | neg |
| BrCa10035 | Tumor | RASGEF1A-HNRNPF | 22 | pos | neg | pos |

TABLE 2
Cell lines

| | | | | | | | | | # |
|---|---|---|---|---|---|---|---|---|---|
| Sample | | Status | Fusions | ER^{a} | PR^{a} | ERBB2^{a} | Source | Culture media | Fusions |
| 1 | BT-20 | Cancer | NOTCH1- | − | − | − | ATCC | DMEM + 10% FBS | 3 |
| | | | GABBR2 | | | | | | |
| 2 | BT-474 | Cancer | | + | + | + | ATCC | RPMI1640 + 10% FBS | 17 |
| 3 | BT-483 | Cancer | | + | + | − | ATCC | RPMI1640 + 10% FBS | 1 |
| 4 | BT-549 | Cancer | | − | − | − | ATCC | RPMI1640 + 10% FBS | 1 |
| 5 | CAL-148 | Cancer | | − | − | − | Reis-Filho | DMEM + 20% FBS | 2 |
| | | | | | | | Lab^{b} | | |
| 6 | CAMA-1 | Cancer | | + | + | − | ATCC | DMEM + 10% FBS | 2 |
| 7 | EFM-19 | Cancer | | + | + | − | Reis-Filho | RPMI1640 + 10% FBS | 7 |
| | | | | | | | Lab^{b} | | |
| 8 | HCC1008 | Cancer | | − | − | + | ATCC | DMEM/F12 + 10% FBS | 20 |
| 9 | HCC1143 | Cancer | | − | − | − | ATCC | RPMI1640 + 10% FBS | 2 |
| 10 | HCC1187 | Cancer | SEC22B- | − | − | − | ATCC | RPMI1640 + 10% FBS | 6 |
| | | | NOTCH2 | | | | | | |
| 11 | HCC1395 | Cancer | | − | − | − | ATCC | RPMI1640 + 10% FBS | 6 |
| 12 | HCC1419 | Cancer | | + | − | + | ATCC | RPMI1640 + 10% FBS | 8 |
| 13 | HCC1428 | Cancer | | + | + | − | ATCC | RPMI1640 + 10% FBS | 8 |
| 14 | HCC1500 | Cancer | | + | + | − | ATCC | RPMI1640 + 10% FBS | 2 |
| 15 | HCC1569 | Cancer | | − | − | + | ATCC | RPMI1640 + 10% FBS | 7 |
| 16 | HCC1599 | Cancer | NOTCH1 | − | − | − | ATCC | RPMI1640 + 10% FBS | 5 |
| | | | internal | | | | | | |
| | | | deletion | | | | | | |
| 17 | HCC1806 | Cancer | | − | − | − | ATCC | RPMI1640 + 10% FBS | 4 |
| 18 | HCC1937 | Cancer | | − | − | − | ATCC | RPMI1640 + 10% FBS | 2 |
| 19 | HCC1954 | Cancer | | − | − | + | ATCC | RPMI1640 + 10% FBS | 3 |
| 20 | HCC202 | Cancer | | − | − | + | ATCC | RPMI1640 + 10% FBS | 1 |
| 21 | HCC2157 | Cancer | | − | − | − | ATCC | RPMI1640 + 10% FBS | 18 |
| 22 | HCC2218 | Cancer | SEC16A- | − | − | + | ATCC | RPMI1640 + 10% FBS | 6 |
| | | | NOTCH1 | | | | | | |
| 23 | HCC38 | Cancer | NOTCH2- | − | − | − | ATCC | RPMI1640 + 10% FBS | 16 |
| | | | SEC22B | | | | | | |
| 24 | HCC70 | Cancer | | − | − | − | ATCC | RPMI1640 + 10% FBS | 2 |
| 25 | Hs 578T | Cancer | | − | − | − | ATCC | DMEM + 10% FBS | 1 |
| 26 | MCF7 | Cancer | | + | + | − | ATCC | DMEM + 10% FBS | 18 |
| 27 | MDA-MB-134-VI | Cancer | | + | − | − | ATCC | DMEM + 10% FBS | 2 |
| 28 | MDA-MB-157 | Cancer | | − | − | − | ATCC | DMEM + 10% FBS | 4 |
| 29 | MDA-MB-175-VII | Cancer | | + | − | − | ATCC | DMEM + 10% FBS | 1 |
| 30 | MDA-MB-330 | Cancer | | + | − | + | ATCC | RPMI1640 + 20% FBS | 1 |
| 31 | MDA-MB-361 | Cancer | | + | + | + | ATCC | DMEM + 10% FBS | 4 |
| 32 | MDA-MB-415 | Cancer | | + | − | − | ATCC | DMEM + 10% FBS | 4 |
| 33 | MDA-MB-453 | Cancer | | − | − | + | ATCC | DMEM + 10% FBS | 2 |
| 34 | MDA-MB-468 | Cancer | ARID1A- | − | − | − | ATCC | RPMI1640 + 10% FBS | 4 |
| | | | MAST2 | | | | | | |
| 35 | SUM149PT | Cancer | | − | − | − | Ethier Lab^{c} | Ham's F12, 5%-IH | 2 |
| 36 | SUM190PT | Cancer | | − | − | + | Ethier Lab^{c} | Ham's F12, SF-IH | 11 |
| 37 | T-47D | Cancer | | + | + | − | ATCC | RPMI1640 + 10% FBS | 3 |
| 38 | UACC-812 | Cancer | | + | + | + | ATCC | RPMI1640 + 10% FBS | 5 |
| 39 | UACC-893 | Cancer | | − | − | + | ATCC | RPMI1640 + 10% FBS | 5 |
| 40 | ZR-75-1 | Cancer | | + | + | − | ATCC | RPMI1640 + 10% FBS | 3 |
| 41 | ZR-75-30 | Cancer | | + | − | + | ATCC | RPMI1640 + 10% FBS | 6 |
| 42 | H16N2 | Normal | | − | − | − | Ethier Lab^{c} | MEBM + supplements: | 0 |
| | | | | | | | | 10% CO2 | |
| 43 | HBL100 | Normal | | − | − | − | Kinch Lab^{d} | DMEM + 10% FBS | 0 |
| 44 | HMEC-1 | Normal | | − | − | − | Lonza | MEBM + supplements | 0 |
| 45 | HMEC-2 | Normal | | − | − | − | Invitrogen | HuMEC Ready Medium | 0 |
| 46 | hTERT-HME1 | Normal | | − | − | − | ATCC | MEBM + supplements | 0 |
| 47 | MCF10A | Normal | | − | − | − | ATCC | MEBM + Lonza | 0 |
| | | | | | | | | supplements | |
| 48 | MCF10F | Normal | | − | − | − | ATCC | DMEM/F12 + supplements | 0 |
| 49 | MCF12A | Normal | | − | − | − | ATCC | DMEM/F12 + supplements | 0 |

Breast tissues

| | | | | | | | | # |
|---|---|---|---|---|---|---|---|---|
| Sample | | Status | Fusions | ER^{a} | PR^{a} | ERBB2^{a} | Source | Fusions |
| 1 | BrBe10001 | Normal | | − | − | − | U Michigan | 0 |
| 2 | BrBe10003 | Normal | | − | − | − | U Michigan | 0 |
| 3 | BrCa00001 | Tumor | ZNF700-MAST1 | + | − | − | U Michigan | 3 |
| 4 | BrCa00002 | Tumor | | + | + | − | U Michigan | 2 |
| 5 | BrCa00003 | Tumor | | − | − | + | U Michigan | 1 |
| 6 | BrCa00004 | Tumor | | + | + | + | U Michigan | 1 |
| 7 | BrCa00005 | Tumor | | − | − | − | U Michigan | 1 |
| 8 | BrCa00006 | Tumor | | + | − | − | U Michigan | 1 |
| 9 | BrCa00007 | Tumor | | + | + | − | U Michigan | 2 |
| 10 | BrCa10001 | Tumor | | + | + | − | U Michigan | 3 |
| 11 | BrCa10002 | Tumor | NOTCH1-Chr9: | − | − | − | U Michigan | 2 |
| | | | 138722663 | | | | | |
| 12 | BrCa10003 | Tumor | | − | − | − | U Michigan | 1 |
| 13 | BrCa10005 | Tumor | | + | − | − | U Michigan | 6 |
| 14 | BrCa10006 | Tumor | | + | + | − | U Michigan | 2 |
| 15 | BrCa10007 | Tumor | | + | + | − | U Michigan | 4 |
| 16 | BrCa10008 | Tumor | | − | − | − | U Michigan | 11 |
| 17 | BrCa10009 | Tumor | | + | + | − | U Michigan | 4 |
| 18 | BrCa10010 | Tumor | | + | + | − | U Michigan | 1 |
| 19 | BrCa10011 | Tumor | | − | − | − | U Michigan | 7 |
| 20 | BrCa10014 | Tumor | | + | + | − | U Michigan | 1 |
| 21 | BrCa10015 | Tumor | | + | − | + | U Michigan | 2 |
| 22 | BrCa10016 | Tumor | | + | − | − | U Michigan | 4 |
| 23 | BrCa10017 | Tumor | NFIX-MAST1 | − | − | + | U Michigan | 8 |
| 24 | BrCa10018 | Tumor | | + | − | − | U Michigan | 5 |
| 25 | BrCa10020 | Tumor | | + | − | − | U Michigan | 3 |
| 26 | BrCa10021 | Tumor | | + | + | − | U Michigan | 4 |
| 27 | BrCa10025 | Tumor | | − | − | − | Reis-Filho Lab^{b} | 7 |
| 28 | BrCa10026 | Tumor | | + | − | + | Reis-Filho Lab^{b} | 8 |
| 29 | BrCa10027 | Tumor | | + | + | − | Reis-Filho Lab^{b} | 8 |
| 30 | BrCa10028 | Tumor | | + | + | − | Reis-Filho Lab^{b} | 6 |
| 31 | BrCa10029 | Tumor | | + | + | − | Reis-Filho Lab^{b} | 13 |
| 32 | BrCa10030 | Tumor | | + | − | + | Reis-Filho Lab^{b} | 7 |
| 33 | BrCa10031 | Tumor | | + | + | − | Reis-Filho Lab^{b} | 0 |
| 34 | BrCa10032 | Tumor | | + | + | − | Reis-Filho Lab^{b} | 0 |
| 35 | BrCa10033 | Tumor | NOTCH1-SNHG7 | − | − | − | Origene | 4 |
| 36 | BrCa10034 | Tumor | | − | − | − | Origene | 0 |
| 37 | BrCa10035 | Tumor | | + | − | + | Origene | 9 |
| 38 | BrCa10036 | Tumor | | − | − | − | Origene | 3 |
| 39 | BrCa10037 | Tumor | | − | − | − | Origene | 3 |

^{a}The ER/PR positivity and ERBB2 overexpression status are derived from RNA sequencing data presented in this study.
^{b}Dr. Jorge Reis-Filho, The Breakthrough Breast Cancer Research Centre, Institute of Cancer Research, London, UK.
^{c}Dr. Stephen Ethier, Karmonos Cancer Institute, Detroit, MI.
^{d}Dr. Michael Kinch, Basic Medical Science, Purdue University.

TABLE 3

| | | Status | Total Count | PF Count | Mapped Reads | Platform |
|---|---|---|---|---|---|---|
| | Cell lines | | | | | |
| 1 | BT-20 | Cancer | 11881181 | 7879015 | 6577574 | GA II |
| 2 | BT-474 | Cancer | 11728387 | 9472843 | 8145601 | GA II |
| 3 | BT-483 | Cancer | 9030162 | 7798595 | 6812699 | GA II |
| 4 | BT-549 | Cancer | 11640006 | 8203409 | 6856696 | GA II |
| 5 | CAL-148 | Cancer | 170185242 | 115495371 | 78967006 | HiSeq 2000 |
| 6 | CAMA-1 | Cancer | 7837225 | 6806921 | 5377266 | GA II |
| 7 | EFM-19 | Cancer | 163672461 | 115237215 | 78000997 | HiSeq 2000 |
| 8 | HCC1008 | Cancer | 22686263 | 16753241 | 14583682 | GA II |
| 9 | HCC1143 | Cancer | 14593087 | 10308363 | 8722464 | GA II |
| 10 | HCC1187 | Cancer | 15811517 | 11110453 | 9461781 | GA II |
| 11 | HCC1395 | Cancer | 14008114 | 10516608 | 9017035 | GA II |
| 12 | HCC1419 | Cancer | 20985359 | 9913182 | 8359909 | GA II |
| 13 | HCC1428 | Cancer | 18670237 | 11765186 | 10008754 | GA II |
| 14 | HCC1500 | Cancer | 14004607 | 12671255 | 11125995 | GA II |
| 15 | HCC1569 | Cancer | 10280279 | 8664918 | 7406087 | GA II |
| 16 | HCC1599 | Cancer | 23513773 | 16997748 | 14666420 | GA II |
| 17 | HCC1606 | Cancer | 9932223 | 6246758 | 5158419 | GA II |
| 18 | HCC1937 | Cancer | 11819695 | 9295846 | 7043676 | GA II |
| 19 | HCC1954 | Cancer | 9936387 | 7095676 | 5941813 | GA II |
| 20 | HCC202 | Cancer | 15939075 | 14059890 | 12110957 | GA II |
| 21 | HCC2157 | Cancer | 19054120 | 15939475 | 13936282 | GA II |
| 22 | HCC2218 | Cancer | 9434541 | 8108277 | 6964856 | GA II |
| 23 | HCC36 | Cancer | 10075786 | 8835013 | 7595252 | GA II |
| 24 | HCC70 | Cancer | 10258297 | 8167896 | 6611373 | GA II |
| 25 | Hs 578T | Cancer | 11782173 | 6637857 | 5438277 | GA II |
| 26 | MCF7 | Cancer | 14448057 | 10580431 | 8220004 | GA II |
| 27 | MDA-MB-134-VI | Cancer | 11753778 | 9389202 | 8028831 | GA II |
| 28 | MDA-MB-157 | Cancer | 10112388 | 8483903 | 7195992 | GA II |
| 29 | MDA-MB-175-VII | Cancer | 11352990 | 8504226 | 6954696 | GA II |
| 30 | MDA-MB-330 | Cancer | 17519050 | 9187752 | 7786389 | GA II |
| 31 | MDA-MB-361 | Cancer | 10623692 | 8336884 | 7033971 | GA II |
| 32 | MDA-MB-415 | Cancer | 10348488 | 9349822 | 8231210 | GA II |
| 33 | MDA-MB-453 | Cancer | 9779378 | 7798963 | 6600811 | GA II |
| 34 | MDA-MB-468 | Cancer | 13323321 | 10379933 | 8792002 | GA II |
| 35 | SUM149PT | Cancer | 16612413 | 14565562 | 12661787 | GA II |
| 36 | SUM190PT | Cancer | 12203177 | 10966281 | 9491706 | GA II |
| 37 | T-47D | Cancer | 12754073 | 9789340 | 8322158 | GA II |
| 38 | UACC-812 | Cancer | 20054278 | 9272801 | 7887775 | GA II |
| 39 | UACC-893 | Cancer | 20008814 | 8795886 | 7592204 | GA II |
| 40 | ZR-75-1 | Cancer | 10946793 | 8901027 | 7539147 | GA II |
| 41 | ZR-75-30 | Cancer | 17310015 | 11763778 | 10035554 | GA II |
| 42 | H16N2 | Normal | 13638417 | 8313731 | 7063742 | GA II |
| 43 | HBL100 | Normal | 9932223 | 6246758 | 5158419 | GA II |
| 44 | HMEC-1 | Normal | 9606020 | 8168925 | 6886202 | GA II |
| 45 | HMEC-2 | Normal | 14131828 | 7884438 | 6840467 | GA II |
| 46 | hTERT-HME1 | Normal | 12991081 | 7844191 | 6654504 | GA II |
| 47 | MCF10A | Normal | 11309743 | 9257186 | 7848773 | GA II |
| 48 | MCF10F | Normal | 11761525 | 8633746 | 7229485 | GA II |
| 49 | MCF12A | Normal | 11601281 | 9479970 | 8043904 | GA II |
| | Breast tissues | | | | | |
| 1 | BrBe10001 | Normal | 9494877 | 8174421 | 7174399 | GA II |
| 2 | BrBe10003 | Normal | 12960276 | 10714939 | 9270382 | GA II |
| 3 | BrCa00001 | Tumor | 16906901 | 11638086 | 9985150 | GA II |
| 4 | BrCa00002 | Tumor | 9548547 | 7917384 | 6949423 | GA II |
| 5 | BrCa00003 | Tumor | 11281870 | 9132553 | 8000217 | GA II |
| 6 | BrCa00004 | Tumor | 14008114 | 10516608 | 9017035 | GA II |
| 7 | BrCa00005 | Tumor | 15274660 | 9935485 | 7528063 | GA II |
| 8 | BrCa00006 | Tumor | 20018598 | 11984485 | 10522998 | GA II |
| 9 | BrCa00007 | Tumor | 11613062 | 9558816 | 8438221 | GA II |
| 10 | BrCa10001 | Tumor | 146079441 | 112808987 | 111442082 | Hiseq 2000 |
| 11 | BrCa10002 | Tumor | 132186880 | 106304457 | 105320759 | Hiseq 2000 |
| 12 | BrCa10003 | Tumor | 145728481 | 112045154 | 110891154 | Hiseq 2000 |
| 13 | BrCa10005 | Tumor | 135668301 | 107577736 | 106391999 | Hiseq 2000 |
| 14 | BrCa10006 | Tumor | 145298113 | 111670856 | 110359851 | Hiseq 2000 |
| 15 | BrCa10007 | Tumor | 139967907 | 109585279 | 108342743 | Hiseq 2000 |
| 16 | BrCa10008 | Tumor | 115590620 | 95588079 | 94280365 | Hiseq 2000 |
| 17 | BrCa10009 | Tumor | 108012117 | 90186492 | 89501918 | Hiseq 2000 |
| 18 | BrCa10010 | Tumor | 117174623 | 95014045 | 94232749 | Hiseq 2000 |
| 19 | BrCa10011 | Tumor | 128989819 | 103689828 | 102928625 | Hiseq 2000 |
| 20 | BrCa10012 | Tumor | 108984765 | 91361240 | 90637466 | Hiseq 2000 |
| 21 | BrCa10014 | Tumor | 83858222 | 74862166 | 74313303 | Hiseq 2000 |
| 22 | BrCa10015 | Tumor | 82029561 | 73387500 | 72812100 | Hiseq 2000 |
| 23 | BrCa10016 | Tumor | 86070198 | 76528467 | 75927905 | Hiseq 2000 |
| 24 | BrCa10017 | Tumor | 81280290 | 73137330 | 72492279 | Hiseq 2000 |
| 25 | BrCa10018 | Tumor | 84674315 | 75937046 | 75428201 | Hiseq 2000 |
| 26 | BrCa10020 | Tumor | 57044679 | 52750471 | 52194523 | Hiseq 2000 |
| 27 | BrCa10025 | Tumor | 70387665 | 64249127 | 63903709 | Hiseq 2000 |
| 28 | BrCa10026 | Tumor | 89603619 | 80206611 | 79388322 | Hiseq 2000 |
| 29 | BrCa10027 | Tumor | 115972826 | 99609129 | 98537660 | Hiseq 2000 |
| 30 | BrCa10028 | Tumor | 110568334 | 95979049 | 94986917 | Hiseq 2000 |
| 31 | BrCa10029 | Tumor | 121828988 | 103645232 | 102445145 | Hiseq 2000 |
| 32 | BrCa10030 | Tumor | 124964884 | 105727507 | 104873235 | Hiseq 2000 |
| 33 | BrCa10031 | Tumor | 120956963 | 102508028 | 101335908 | Hiseq 2000 |
| 34 | BrCa10032 | Tumor | 123273262 | 104313810 | 103452180 | Hiseq 2000 |
| 35 | BrCa10033 | Tumor | 185362673 | 100983404 | 69389326 | HiSeq 2000 |
| 36 | BrCa10034 | Tumor | 166152555 | 115282565 | 73715041 | HiSeq 2000 |
| 37 | BrCa10035 | Tumor | 164039623 | 117114397 | 79565960 | HiSeq 2000 |
| 38 | BrCa10036 | Tumor | 160117837 | 115875880 | 78972078 | HiSeq 2000 |
| 39 | BrCa10037 | Tumor | 148280237 | 111548797 | 79339795 | HiSeq 2000 |

PF = Pass filter

TABLE 4

| | | | | | | | |
|---|---|---|---|---|---|---|---|
| | | | | | | Vali- | |
| | | | | Se- | | dation | |
| Sample | 5′ | 3′ | | quencing | # | Fusion | Chromosomal Location |
| Name | Gene | Gene | Type | Platform | Reads | qPCR | 5′ Gene |

Breast cell lines

| | | | | | | | |
|---|---|---|---|---|---|---|---|
| BT-20 | NOTCH1 | GABBR2 | Intra | GA II | 21 | Y | chr9: 138508717-138560059 |
| BT-20 | GOLGB1 | ILDR1 | Intra | GA II | 14 | Y | chr3: 122864737-122951292 |
| BT-20 | PLEKHB2 | ARHGEF4 | Intra | GA II | 6 | Y | chr2: 131578889-131623895 |
| BT-474 | RPS6KB1 | SNF8 | Intra | GA II | 92 | Y | chr17: 55325224-55382568 |
| BT-474 | STX16 | RAE1 | Intra | GA II | 79 | Y | chr20: 56659733-56687988 |
| BT-474 | ZMYND8 | CEP250 | Intra | GA II | 77 | Y | chr20: 45271787-45418881 |
| BT-474 | TRPC4AP | MRPL45 | Inter | GA II | 30 | | chr20: 33053867-33144279 |
| BT-474 | MED1 | STXBP4 | Intra | GA II | 28 | Y | chr17: 34814063-34861053 |
| BT-474 | TOB1 | AP1GBP1 | Intra | GA II | 16 | | chr17: 46294585-46296412 |
| BT-474 | ACACA | STAC2 | Intra | GA II | 15 | | chr17: 32516039-32841015 |
| BT-474 | MED13 | BCAS3 | Intra | GA II | 13 | Y | chr17: 57374747-57497425 |
| BT-474 | VAPB | IKZF3 | Inter | GA II | 13 | Y | chr20: 56397580-56459562 |
| BT-474 | RAB22A | MYO9B | Inter | GA II | 9 | Y | chr20: 56318176-56375969 |
| BT-474 | GLB1 | CMTM7 | Intra | GA II | 7 | | chr3: 33013103-33113698 |
| BT-474 | NCOA2 | ZNF704 | Intra | GA II | 7 | Y | chr8: 71186820-71478574 |
| BT-474 | BCAS3 | MED13 | Intra | GA II | 6 | | chr17: 56109953-56824981 |
| BT-474 | PIP4K2B | RAD51C | Intra | GA II | 6 | | chr17: 34175469-34209684 |
| BT-474 | PPP1R12A | MGAT4C | Intra | GA II | 6 | | chr12: 78691473-78853366 |
| BT-474 | STARD3 | DOCK5 | Inter | GA II | 6 | | chr17: 35046858-35073980 |
| BT-474 | TRIM37 | MYO19 | Intra | GA II | 6 | | chr17: 54414781-54539048 |
| BT-483 | SMARCB1 | MARK3 | Inter | GA II | 7 | Y | chr22: 22459149-22506705 |
| BT-549 | CLTC | TMEM49 | Intra | GA II | 18 | Y | chr17: 55051831-55129099 |
| CAL-148 | SSR2 | ERRFI1 | Intra | HiSeq 2000 | 28 | | chr1: 154,245,463-154,257,382 |
| CAL-148 | CELSR3 | IP6K1 | Intra | HiSeq 2000 | 10 | | chr3: 48,648,900-48,675,352 |
| CAMA-1 | ST7 | PRKAG2 | Intra | GA II | 8 | Y | chr7: 116380616-116657311 |
| CAMA-1 | PLDN | SQRDL | Intra | GA II | 5 | Y | chr15: 43666708-43689201 |
| EFM-19 | FBRS | ZNF771 | Intra | HiSeq 2000 | 386 | | chr16: 30,583,279-30,589,632 |
| EFM-19 | ZFYVE9 | USP33 | Intra | HiSeq 2000 | 95 | | chr1: 52,380,634-52,584,946 |
| EFM-19 | BCAS3 | TG | Intra | HiSeq 2000 | 74 | | chr17: 56,109,954-56,824,981 |
| EFM-19 | KIRREL | ZFYVE9 | Intra | HiSeq 2000 | 86 | | chr1: 156,229,687-156,332,468 |
| EFM-19 | ZCCHC7 | C9orf25 | Intra | HiSeq 2000 | 50 | | chr9: 37,110,469-37,348,145 |
| EFM-19 | CRLF3 | CHD9 | Intra | HiSeq 2000 | 35 | | chr17: 26,133,828-26,175,904 |
| EFM-19 | USP54 | ZMIZ1 | Intra | HiSeq 2000 | 26 | | chr10: 74,927,302-75,005,439 |
| HCC1008 | RFX1 | ASNA1 | Intra | GA II | 210 | | chr19: 13933352-13978097 |
| HCC1008 | CBX7 | ENTHD1 | Intra | GA II | 45 | | chr22: 37856725-37878484 |
| HCC1008 | CCDC117 | HSCB | Intra | GA II | 23 | | chr22: 27498707-27515278 |
| HCC1008 | RHOA | WWTR1 | Intra | GA II | 17 | | chr3: 49371583-49424530 |
| HCC1008 | FITM2 | FAM193A | Inter | GA II | 17 | | chr20: 42368611-42373303 |
| HCC1008 | HTT | ADD1 | Intra | GA II | 15 | | chr4: 3046206-3215485 |
| HCC1008 | RASAL1 | CDC42BPA | Intra | GA II | 15 | | chr12: 112021701-112058404 |
| HCC1008 | C10ORF18 | NET1 | Intra | GA II | 12 | | chr10: 5766807-5846949 |
| HCC1008 | RPS6KA1 | RHOC | Intra | GA II | 10 | | chr1: 26744930-26774107 |
| HCC1008 | ST3GAL4 | DCPS | Intra | GA II | 10 | | chr11: 125731306-125789743 |
| HCC1008 | FARS2 | CDYL | Intra | GA II | 10 | | chr6: 5206583-5716815 |
| HCC1008 | CIRH1A | CDH1 | Intra | GA II | 10 | | chr16: 67724000-67760438 |
| HCC1008 | EU154352 | PLXNA2 | Intra | GA II | 9 | | chr1: 206041490-206062671 |
| HCC1008 | YWHAQ | ITPRIPL1 | Intra | GA II | 8 | | chr2: 9641557-9688557 |
| HCC1008 | SLC9A1 | RERE | Intra | GA II | 7 | | chr1: 27297894-27353988 |
| HCC1008 | ZNF430 | PPIG | Inter | GA II | 4 | | chr19: 20995337-21033493 |
| HCC1008 | MAGI1 | STMN2 | Inter | GA II | 4 | | chr3: 65314946-65999549 |
| HCC1008 | NOL5A | TMC2 | Intra | GA II | 3 | | chr20: 2581488-2585538 |
| HCC1008 | CRYBB2 | KIAA1671 | Intra | GA II | 3 | | chr22: 23945612-23957836 |
| HCC1008 | CDYL | RERE | Inter | GA II | 3 | | chr6: 4721682-4900777 |
| HCC1143 | C18orf45 | HM13 | Inter | GA II | 25 | Y | chr18: 19129977-19271923 |
| HCC1143 | C2ORF48 | RRM2 | Intra | GA II | 23 | Y | chr2: 10198959-10269307 |
| HCC1187 | PUM1 | TRERF1 | Inter | GA II | 38 | Y | chr1: 31176939-31311151 |
| HCC1187 | SEC22B | NOTCH2 | Intra | GA II | 30 | Y | chr1: 143807763-143828279 |
| HCC1187 | CTAGE5 | SIP1 | Intra | GA II | 15 | | chr14: 38806079-38890148 |
| HCC1187 | MCPH1 | AGPAT5 | Intra | GA II | 11 | | chr8: 6251520-6488548 |
| HCC1187 | KLK5 | CDH23 | Inter | GA II | 5 | | chr19: 56138370-56148156 |
| HCC1187 | BC041478 | EXOSC10 | Inter | GA II | 3 | | chr19: 42434668-42446354 |
| HCC1395 | EIF3K | CYP39A1 | Inter | GA II | 13 | Y | chr19: 43801561-43819435 |
| HCC1395 | HNRNPUL2 | AHNAK | Intra | GA II | 13 | Y | chr11: 62238795-62251397 |
| HCC1395 | RAB7A | LRCH3 | Intra | GA II | 6 | | chr3: 129927668-130016331 |
| HCC1395 | ERO1L | FERMT2 | Intra | GA II | 5 | | chr14: 52178354-52232169 |
| HCC1395 | FOSL2 | BRE | Intra | GA II | 5 | | chr2: 28469282-28491020 |
| HCC1395 | BCAR3 | ABCA4 | Intra | GA II | 4 | | chr1: 93799936-93919973 |
| HCC1419 | PLEC1 | C8ORF38 | Intra | GA II | 174 | | chr8: 145061309-145122889 |
| HCC1419 | VPS18 | ZFYVE19 | Intra | GA II | 78 | | chr15: 38973920-38983465 |
| HCC1419 | CCNE2 | FAM82B | Intra | GA II | 49 | | chr8: 95961629-95976658 |
| HCC1419 | STARD3 | TAC4 | Intra | GA II | 27 | | chr17: 35046858-35073980 |
| HCC1419 | VAV2 | TRUB2 | Intra | GA II | 18 | | chr9: 135616836-135847267 |
| HCC1419 | EIF3H | FAM65C | Inter | GA II | 11 | | chr8: 117726236-117837243 |
| HCC1419 | ZNF251 | TSHZ2 | Inter | GA II | 9 | | chr8: 145917102-145951775 |
| HCC1419 | RAE1 | NFKBIL2 | Inter | GA II | 4 | | chr20: 55360025-55386926 |
| HCC1428 | SPAG9 | NGFR* | Intra | GA II | 24 | | chr17: 46397987-46553094 |
| HCC1428 | SLC37A1 | ABCG1 | Intra | GA II | 18 | | chr21: 42792811-42874619 |
| HCC1428 | ESR1 | C6ORF97 | Intra | GA II | 13 | | chr6: 152170147-152466101 |
| HCC1428 | RNF187 | OBSCN | Intra | GA II | 6 | | chr1: 226741690-226750512 |
| HCC1428 | CDK5RAP2 | MEGF9 | Intra | GA II | 6 | | chr9: 122190968-122382258 |
| HCC1428 | LUZP1 | BC041441 | Intra | GA II | 4 | | chr1: 23284038-23368104 |
| HCC1428 | ZNF362 | ROR1 | Intra | GA II | 4 | | chr1: 33494761-33538907 |
| HCC1428 | UNQ2998 | OPRD1 | Intra | GA II | 4 | | chr1: 1007061-1017346 |
| HCC1500 | SLC9A7 | ALDH7A1 | Inter | GA II | 18 | | chrX: 46351317-46503416 |
| HCC1500 | CHN2 | RALY | Inter | GA II | 3 | | chr7: 29200646-29520469 |
| HCC1569 | PTPRJ | LPXN | Intra | GA II | 53 | | chr11: 47958685-48110842 |
| HCC1569 | RFT1 | UQCRC2 | Inter | GA II | 53 | | chr3: 53097540-53139510 |
| HCC1569 | TMEM189 | GMDS | Inter | GA II | 28 | | chr20: 48173680-48203742 |
| HCC1569 | LDLRAD3 | TCP11L1 | Intra | GA II | 20 | | chr11: 35922187-36209417 |
| HCC1569 | SMURF2 | CCDC46 | Intra | GA II | 7 | | chr17: 59971196-60088848 |
| HCC1569 | PPP1R1B | STARD3 | Intra | GA II | 6 | Y | chr17: 35038278-35046404 |
| HCC1569 | PSD3 | CHGN | Intra | GA II | 6 | Y | chr8: 18429092-18710685 |
| HCC1599 | EXOC7 | CYTH1 | Intra | GA II | 42 | | chr17: 71588680-71611463 |
| HCC1599 | PSCD1 | PRPSAP1 | Intra | GA II | 31 | Y | chr17: 74181724-74289971 |
| HCC1599 | MSL2L1 | SFRS10 | Intra | GA II | 7 | | chr3: 137350450-137397378 |
| HCC1599 | PPAT | AASDH | Intra | GA II | 5 | | chr4: 56954285-56996602 |
| HCC1599 | TIPARP | LEKR1 | Intra | GA II | 4 | | chr3: 157875408-157907251 |
| HCC1806 | POLA2 | CAPN1 | Intra | GA II | 28 | Y | chr11: 64786007-64821664 |
| HCC1806 | TAX1BP1 | AHCY | Inter | GA II | 21 | Y | chr7: 27746262-27835911 |
| HCC1806 | WNK1 | CWC22 | Inter | GA II | 16 | | chr12: 732349-890879 |
| HCC1806 | WNK1 | USP31 | Inter | GA II | 4 | | chr12: 732349-890879 |
| HCC1937 | NFIA | EHF | Inter | GA II | 4 | | chr1: 61320883-61694624 |
| HCC1937 | RNF121 | SFRS2IP | Inter | GA II | 4 | | chr11: 71317731-71386291 |
| HCC1954 | C6orf106 | SPDEF | Intra | GA II | 24 | Y | chr6: 34663048-34772603 |
| HCC1954 | INTS1 | PRKAR1B | Intra | GA II | 22 | Y | chr7: 1476438-1510544 |
| HCC1954 | GALNT7 | ORC4L | Inter | GA II | 9 | | chr4: 174326478-174481693 |
| HCC202 | FBXL20 | SNF8 | Intra | GA II | 78 | Y | chr17: 34662422-34811435 |
| HCC2157 | PSMD3 | PPP1R1B | Intra | GA II | 74 | | chr17: 35390586-35407738 |
| HCC2157 | KIAA0515 | PPAPDC3 | Intra | GA II | 63 | | chr9: 133295298-133365396 |
| HCC2157 | SMYD3 | ZNF670 | Intra | GA II | 52 | | chr1: 243979267-244647334 |
| HCC2157 | RBM14 | PACS1 | Intra | GA II | 38 | | chr11: 66140673-66151389 |
| HCC2157 | THRAP3 | EIF2C3 | Intra | GA II | 28 | | chr1: 36462604-36543544 |
| HCC2157 | NUDT3 | BRPF3 | Intra | GA II | 23 | | chr6: 34363975-34468419 |
| HCC2157 | RASA2 | ACPL2 | Intra | GA II | 15 | | chr3: 142688616-142813887 |
| HCC2157 | RANBP1 | C22orf25 | Intra | GA II | 14 | | chr22: 18485024-18494704 |
| HCC2157 | AXIN1 | LMF1 | Inter | GA II | 11 | | chr16: 277441-342465 |
| HCC2157 | ASCC1 | CBARA1 | Intra | GA II | 11 | | chr10: 73526284-73645700 |
| HCC2157 | CORO7 | VPS13D | Inter | GA II | 11 | | chr16: 4344544-4406963 |
| HCC2157 | DNMT1 | KEAP1 | Intra | GA II | 8 | | chr19: 10105022-10166811 |
| HCC2157 | PRMT7 | SLC7A6 | Intra | GA II | 5 | | chr16: 66902446-66948663 |
| HCC2157 | PSPC1 | FAM179A | Inter | GA II | 5 | | chr13: 19175009-19255083 |
| HCC2157 | RGS3 | SLC31A2 | Intra | GA II | 4 | | chr9: 115246832-115399839 |
| HCC2157 | ZNF236 | ZNF516 | Intra | GA II | 4 | | chr18: 72665104-72811670 |
| HCC2157 | FRYL | OCIAD2 | Intra | GA II | 4 | | chr4: 48194137-48477073 |
| HCC2157 | BAZ1A | SNX6 | Intra | GA II | 3 | | chr14: 34291688-34414604 |
| HCC2218 | SEC16A | NOTCH1 | Intra | GA II | 14 | Y | chr9: 138454368-138497328 |
| HCC2218 | POLDIP2 | BRIP1 | Intra | GA II | 8 | | chr17: 23697785-23708730 |
| HCC2218 | INTS2 | ZNF652 | Intra | GA II | 7 | | chr17: 57297509-57360159 |
| HCC2218 | INTS2 | TMEM49 | Intra | GA II | 5 | | chr17: 57297509-57360159 |
| HCC2218 | LRRC59 | NEUROD2 | Intra | GA II | 5 | | chr17: 45813592-45829913 |
| HCC2218 | PERLD1 | PPM1D | Intra | GA II | 4 | Y | chr17: 35082579-35097833 |
| HCC38 | TMEM123 | MMP20 | Intra | GA II | 36 | | chr11: 101772266-101828985 |
| HCC38 | MTAP | PCDH7 | Inter | GA II | 24 | | chr9: 21792635-21855969 |
| HCC38 | HMGXB3 | PPARGC1B | Intra | GA II | 17 | | chr5: 149360362-149412899 |
| HCC38 | RNF111 | TCF12 | Intra | GA II | 16 | | chr15: 57067157-57176545 |
| HCC38 | MED1 | GSDMB | Intra | GA II | 11 | | chr17: 34814064-34861053 |
| HCC38 | SPRED1 | BUB1B | Intra | GA II | 10 | | chr15: 36332343-36436742 |
| HCC38 | NOS1AP | IFI16 | Intra | GA II | 8 | | chr1: 160306205-160606437 |
| HCC38 | MBOAT2 | PRKCE | Intra | GA II | 6 | | chr2: 8914151-9061327 |
| HCC38 | NOTCH2 | SEC22B | Intra | GA II | 6 | Y | chr1: 120255699-120413799 |
| HCC38 | LOC399959 | ZNF202 | Intra | GA II | 5 | | chr11: 121465021-121578980 |
| HCC38 | TBCE | ACTN2 | Intra | GA II | 4 | | chr1: 233597351-233678903 |
| HCC38 | ACBD6 | RRP15 | Intra | GA II | 4 | | chr1: 178523988-178738102 |
| HCC38 | SCAPER | TM6SF1 | Intra | GA II | 4 | | chr15: 74427592-74963272 |
| HCC38 | RBM23 | PSMB5 | Intra | GA II | 3 | | chr14: 22439694-22458236 |
| HCC38 | BCL2L12 | PRMT1 | Intra | GA II | 3 | Y | chr19: 54860210-54868985 |
| HCC38 | FBXL17 | PJA2 | Intra | GA II | 3 | | chr5: 107223348-107745010 |
| HCC70 | C5orf22 | PDCD6 | Intra | GA II | 6 | | chr5: 31568130-31590922 |
| HCC70 | MAP7D1 | ACTB | Inter | GA II | 3 | | chr1: 36394390-36419028 |
| Hs578T | CALD1 | GPATCH4 | Inter | GA II | 3 | | chr7: 134114711-134306012 |
| MCF7 | BCAS4 | BCAS3 | Inter | GA II | 2788 | | chr20: 48844873-48927121 |
| MCF7 | ARFGEF2 | SULF2 | Intra | GA II | 305 | Y | chr20: 46971681-47086637 |
| MCF7 | RPS6KB1 | TMEM49 | Intra | GA II | 78 | Y | chr17: 55325224-55382568 |
| MCF7 | STK11 | MIDN | Intra | GA II | 25 | | chr19: 1156797-1179434 |
| MCF7 | PAPOLA | AK7 | Intra | GA II | 16 | Y | chr14: 96038472-96103201 |
| MCF7 | AHCYL1 | RAD51C | Inter | GA II | 12 | Y | chr1: 110328830-110367887 |
| MCF7 | EIF3H | FAM65C | Inter | GA II | 11 | | chr8: 117726235-117837243 |
| MCF7 | BC017255 | TMEM49 | Intra | GA II | 10 | | chr17: 54538741-54550409 |
| MCF7 | ADAMTS19 | SLC27A6 | Intra | GA II | 9 | | chr5: 128824001-129102275 |
| MCF7 | ARHGAP19 | DRG1 | Inter | GA II | 8 | Y | chr10: 98971919-99042403 |
| MCF7 | MYO9B | FCHO1 | Intra | GA II | 8 | Y | chr19: 17047590-17185104 |
| MCF7 | HSPE1 | PREI3 | Intra | GA II | 6 | Y | chr2: 198072965-198076432 |
| MCF7 | PARD6G | C18ORF1 | Intra | GA II | 6 | | chr18: 76016105-76106388 |
| MCF7 | TRIM37 | TMEM49 | Intra | GA II | 6 | Y | chr17: 54414781-54539048 |
| MCF7 | SMARCA4 | CARM1 | Intra | GA II | 5 | Y | chr19: 10955827-11033958 |
| MCF7 | BCAS4 | ZMYND8 | Intra | GA II | 4 | Y | chr20: 48844873-48927121 |
| MCF7 | PVT1 | MYC | Intra | GA II | 4 | Y | chr8: 128875961-129182681 |
| | (BC041065) | | | | | | |
| MCF7 | TRIM37 | RNFT1 | Intra | GA II | 3 | | chr17: 54414781-54539048 |
| MDA-MB-134 | ANK1 | ZMAT4 | Intra | GA II | 18 | Y | chr8: 41629900-41641961 |
| MDA-MB-134 | BC035340 | MCF2L | Intra | GA II | 15 | Y | chr13: 112604510-112660478 |
| MDA-MB-157 | CCDC9 | KIAA0134 | Intra | GA II | 28 | Y | chr19: 52451570-52467050 |
| MDA-MB-157 | TYRO3 | RTF1 | Intra | GA II | 17 | Y | chr15: 39638511-39658828 |
| MDA-MB-157 | C12ORF49 | ATP10A | Inter | GA II | 16 | | chr12: 115637978-115660226 |
| MDA-MB-157 | UVRAG | MOGAT2 | Intra | GA II | 11 | | chr11: 75203859-75532930 |
| MDA-MB-175VII | SAPS3 | ODZ4 | Intra | GA II | 23 | | chr11: 68029189-68139295 |
| MDA-MB-330 | ACACA | DDX52 | Intra | GA II | 7 | Y | chr17: 32516039-32841015 |
| MDA-MB-361 | TMEM104 | CRKRS | Intra | GA II | 18 | Y | chr17: 70284216-70347517 |
| MDA-MB-361 | TANC1 | MTMR4 | Inter | GA II | 12 | Y | chr2: 159533391-159797416 |
| MDA-MB-361 | TOX3 | GNAO1 | Intra | GA II | 7 | | chr16: 51029418-51139215 |
| MDA-MB-361 | SUPT4H1 | CCDC46 | Intra | GA II | 5 | Y | chr17: 53777537-53784562 |
| MDA-MB-415 | LRP5 | TPCN2 | Intra | GA II | 156 | | chr11: 67836684-67973319 |
| MDA-MB-415 | RAD9A | SHANK2 | Intra | GA II | 27 | | chr11: 66915999-66922459 |
| MDA-MB-415 | SHANK2 | OTUB1 | Intra | GA II | 11 | | chr11: 69991609-70185520 |
| MDA-MB-415 | ZNF331 | ANO1 | Inter | GA II | 13 | | chr19: 58733145-58775335 |
| MDA-MB-453 | MECP2 | TMLHE | Intra | GA II | 8 | | chrX: 152948879-153016382 |
| MDA-MB-453 | MYO15B | MAP3K3 | Intra | GA II | 4 | | chr17: 71095733-71134522 |
| MDA-MB-468 | UBR5 | SLC25A32 | Intra | GA II | 8 | | chr8: 103334744-103493671 |
| MDA-MB-468 | ARID1A | MAST2 | Intra | GA II | 5 | Y | chr1: 26895108-26981188 |
| MDA-MB-468 | EGFR | POLD1 | Inter | GA II | 5 | | chr7: 55054218-55203822 |
| MDA-MB-468 | RDH13 | FBXO3 | Inter | GA II | 3 | | chr19: 60247503-60266397 |
| SUM149PT | EXOSC1 | CRTAC1 | Intra | GA II | 5 | | chr10: 99185655-99195758 |
| SUM149PT | ZDHHC5 | EPB41L5 | Inter | GA II | 5 | | chr11: 57192049-57225235 |
| SUM190PT | NR1D1 | C17ORF75 | Intra | GA II | 37 | | chr17: 35502562-35510499 |
| SUM190PT | PLA2G4A | FAM5C | Intra | GA II | 13 | | chr1: 185064654-185224736 |
| SUM190PT | GPR97 | GPR56 | Intra | GA II | 9 | | chr16: 56259657-56280791 |
| SUM190PT | KLHL22 | CRKL | Intra | GA II | 9 | | chr22: 19125805-19180170 |
| SUM190PT | SGMS2 | PERLD1 | Inter | GA II | 9 | | chr4: 109033868-109055652 |
| SUM190PT | SLC43A1 | FAM168A | Intra | GA II | 9 | | chr11: 57008582-57039735 |
| SUM190PT | LYPD6B | KIF5C | Intra | GA II | 5 | | chr2: 149603226-149780018 |
| SUM190PT | SHANK2 | DKFZP586P0123 | Intra | GA II | 5 | | chr11: 69991608-70185571 |
| SUM190PT | C2CD3 | BC044946 | Inter | GA II | 4 | | chr11: 73423127-73559712 |
| SUM190PT | PERLD1 | CYP2U1 | Inter | GA II | 4 | | chr17: 35082579-35097833 |
| SUM190PT | PROM2 | POLR3GL | Inter | GA II | 4 | | chr2: 95303927-95320782 |
| T-47D | RERG | CBFB | Inter | GA II | 7 | | chr12: 15151984-15265571 |
| T-47D | VGLL4 | SH3BP5 | Intra | GA II | 3 | | chr3: 11572544-11660398 |
| T-47D | NBPF1 | CROCC | Intra | GA II | 3 | | chr1: 16762999-16812569 |
| UACC-812 | HDGF | S100A10 | Intra | GA II | 56 | Y | chr1: 154978522-154988648 |
| UACC-812 | PPP1R12B | SNX27 | Intra | GA II | 22 | Y | chr1: 200584452-200824320 |
| UACC-812 | WIPF2 | HER2 | Intra | GA II | 7 | Y | chr17: 35629099-35691965 |
| UACC-812 | CDC6 | IKZF3 | Intra | GA II | 3 | Y | chr17: 35697671-35712939 |
| UACC-812 | MLLT6 | TEM7 | Intra | GA II | 3 | Y | chr17: 34115398-34139582 |
| UACC-893 | FBXL20 | CRKRS | Intra | GA II | 31 | Y | chr17: 34662422-34811435 |
| UACC-893 | CCDC6 | ANK3 | Intra | GA II | 27 | Y | chr10: 61218511-61336420 |
| UACC-893 | grb7V | PPP1R1B | Intra | GA II | 23 | Y | chr17: 35152031-35157064 |
| UACC-893 | MED1 | IKZF3 | Intra | GA II | 9 | Y | chr17: 34814063-34861053 |
| UACC-893 | EIF2AK3 | PRKD3 | Intra | GA II | 5 | | chr2: 88637373-88708209 |
| ZR-75-1 | FOXJ3 | CAMTA1 | Intra | GA II | 10 | | chr1: 42414796-42573490 |
| ZR-75-1 | GPATCH3 | CAMTA1 | Intra | GA II | 10 | | chr1: 27089565-27099549 |
| ZR-75-1 | C1ORF151 | RCC2 | Intra | GA II | 9 | | chr1: 19796057-19828901 |
| ZR-75-30 | USP32 | CCDC49 | Intra | GA II | 264 | Y | chr17: 55609472-55824368 |
| ZR-75-30 | DDX5 | DEPDC6 | Inter | GA II | 241 | Y | chr17: 59924835-59932946 |
| ZR-75-30 | PLEC1 | ENPP2 | Intra | GA II | 30 | Y | chr8: 145061309-145122889 |
| ZR-75-30 | BCAS3 | HOXB9 | Intra | GA II | 24 | Y | chr17: 56109953-56824981 |
| ZR-75-30 | TAOK1 | PCGF2 | Intra | GA II | 14 | Y | chr17: 24742068-24895628 |
| ZR-75-30 | ERBB2 | BCAS3 | Intra | GA II | 5 | | chr17: 35097918-35138441 |

Breast tumor tissues

| | | | | | | | |
|---|---|---|---|---|---|---|---|
| BrCa00001 | PPP1R14C | C6ORF97 | Intra | GA II | 9 | | chr6: 150505880-150613221 |
| BrCa00001 | ZNF700 | MAST1 | Intra | GA II | 5 | Y | chr19: 11896899-11922578 |
| BrCa00001 | SMURF1 | PDAP1 | Intra | GA II | 3 | | chr7: 98462999-98579659 |
| BrCa00002 | SLC44A2 | PBX4 | Intra | GA II | 9 | Y | chr19: 10597170-10616235 |
| BrCa00002 | WDR68 | PRR11 | Intra | GA II | 8 | Y | chr17: 58981554-59025373 |
| BrCa00003 | KIAA1267 | MBTD1 | Intra | GA II | 25 | Y | chr17: 41463128-41658517 |
| BrCa00004 | MTG1 | FLJ00268 | Intra | GA II | 5 | Y | chr10: 135057610-135084164 |
| BrCa00005 | THOC6 | CLDN9 | Intra | GA II | 28 | | chr16: 3014032-3017757 |
| BrCa00006 | STK3 | RIMS2 | Intra | GA II | 5 | | chr8: 99536036-99907085 |
| BrCa00007 | PSMD3 | LOC284100 | Intra | GA II | 16 | Y | chr17: 35390585-35407738 |
| BrCa00007 | PGCP | C8orf47 | Intra | GA II | 5 | Y | chr8: 97726674-98224898 |
| BrCa10001 | SEC11C | MALT1 | Intra | Hiseq 2000 | 20 | | Chr18: 54958104-54977043 |
| BrCa10001 | SMYD3 | RGS7 | Intra | Hiseq 2000 | 16 | | Chr1: 243979266-244737237 |
| BrCa10001 | C2ORF67 | KIF1A | Intra | Hiseq 2000 | 9 | | chr2: 210593680-210744296 |
| BrCa10002 | NOTCH1 | chr9: 138722683 | Intra | HiSeq 2000 | 14 | Y | chr9: 138508717-138560059 |
| BrCa10002 | RFX3 | DMRT1 | Intra | Hiseq 2000 | 25 | | Chr9: 3214646-3515983 |
| BrCa10003 | VPS13C | TMEM184B | Inter | Hiseq 2000 | 5 | | Chr15: 59931881-60139939 |
| BrCa10005 | TTC3 | RAB22A | Inter | Hiseq 2000 | 216 | | Chr21: 37367440-37497278 |
| BrCa10005 | DIDO1 | C20ORF151 | Intra | Hiseq 2000 | 127 | | Chr20: 60979534-61039719 |
| BrCa10005 | E2F5 | FER1L6 | Intra | Hiseq 2000 | 25 | | Chr8: 86276870-86314006 |
| BrCa10005 | SLC25A26 | CADPS | Intra | Hiseq 2000 | 13 | | Chr3: 66376316-66521220 |
| BrCa10005 | IQCK | EXOD1 | Intra | Hiseq 2000 | 9 | | Chr16: 19635278-19776360 |
| BrCa10005 | MRPS28 | CHRM1 | Inter | Hiseq 2000 | 8 | | Chr8: 80993649-81105061 |
| BrCa10006 | ZNF207 | CCDC102A | Inter | Hiseq 2000 | 321 | | Chr17: 27701269-27732088 |
| BrCa10006 | GPATCH8 | BRIP1 | Intra | Hiseq 2000 | 16 | | Chr17: 39828175-39936328 |
| BrCa10007 | HK1 | MYPN | Intra | Hiseq 2000 | 40 | | Chr10: 70699761-70831643 |
| BrCa10007 | TSPAN15 | EBF3 | Intra | Hiseq 2000 | 20 | | Chr10: 70881231-70937429 |
| BrCa10007 | SSH1 | FAM109A | Intra | Hiseq 2000 | 9 | | Chr12: 107705098-107775480 |
| BrCa10007 | CD151 | TSPAN4 | Intra | Hiseq 2000 | 8 | | Chr11: 822951-828835 |
| BrCa10008 | SQLE | NSMCE2 | Intra | Hiseq 2000 | 86 | | Chr8: 126079900-126103707 |
| BrCa10008 | RFX5 | SELENBP1 | Intra | Hiseq 2000 | 43 | | Chr1: 149579739-149586393 |
| BrCa10008 | KIAA0146 | MCM4 | Intra | Hiseq 2000 | 37 | | Chr8: 48336094-48811028 |
| BrCa10008 | SSBP1 | WEE2 | Intra | Hiseq 2000 | 33 | | Chr7: 141084644-141096726 |
| BrCa10008 | LGALS12 | SYNPO2L | Inter | Hiseq 2000 | 22 | | Chr11: 63030131-63040815 |
| BrCa10008 | ETV6 | SYN1 | Inter | Hiseq 2000 | 16 | | Chr12: 11694054-12159528 |
| BrCa10008 | WNK1 | ERC1 | Intra | Hiseq 2000 | 12 | | Chr12: 732485-890879 |
| BrCa10008 | NSMCE1 | CCDC101 | Intra | Hiseq 2000 | 11 | | Chr16: 27143815-27187614 |
| BrCa10008 | FLNB | LGALS12 | Inter | Hiseq 2000 | 9 | | Chr3: 57969166-58133017 |
| BrCa10008 | RAB40C | C16ORF14 | Inter | Hiseq 2000 | 8 | | Chr16: 580107-619273 |
| BrCa10008 | AX747739 | RHOU | Inter | Hiseq 2000 | 7 | | Chr20: 58146931-58330709 |
| BrCa10009 | ACAD9 | C3ORF46 | Intra | Hiseq 2000 | 36 | | Chr3: 130081022-130117600 |
| BrCa10009 | TESK2 | PRDX1 | Intra | Hiseq 2000 | 15 | | Chr1: 45582141-45729427 |
| BrCa10009 | BC038786 | HPD | Intra | Hiseq 2000 | 8 | | Chr12: 120717559-120725773 |
| BrCa10009 | USP3 | APH1B | Intra | Hiseq 2000 | 7 | | Chr15: 61583862-61670716 |
| BrCa10010 | VPS28 | AK024242 | Intra | Hiseq 2000 | 17 | | Chr8: 145619807-145624735 |
| BrCa10011 | STK3 | ODF1 | Intra | Hiseq 2000 | 145 | | Chr8: 99536036-100024049 |
| BrCa10011 | ZNF638 | C21ORF91 | Inter | Hiseq 2000 | 23 | | Chr2: 71357230-71515697 |
| BrCa10011 | CABLES2 | SNTA1 | Intra | Hiseq 2000 | 22 | | Chr20: 60397080-60415734 |
| BrCa10011 | RMND5B | SFXN1 | Intra | Hiseq 2000 | 8 | | Chr5: 177490633-177508085 |
| BrCa10011 | INPP4A | C4ORF28 | Inter | Hiseq 2000 | 7 | | Chr2: 98427844-98570598 |
| BrCa10011 | PUM1 | SAPS3 | Inter | Hiseq 2000 | 7 | | Chr1: 31176939-31311350 |
| BrCa10011 | VPS13B | POLR2K | Intra | Hiseq 2000 | 7 | | Chr8: 100094669-100958984 |
| BrCa10014 | MACROD1 | VEGFB | Intra | Hiseq 2000 | 101 | | Chr11: 63522605-63690094 |
| BrCa10015 | FRS2 | LYZ | Intra | Hiseq 2000 | 60 | | Chr12: 68150395-68259829 |
| BrCa10015 | BTBD7 | BC016484 | Intra | Hiseq 2000 | 6 | | Chr14: 92773648-92869138 |
| BrCa10016 | RABGGTA | ADCY4 | Intra | Hiseq 2000 | 24 | | Chr14: 23804583-23810643 |
| BrCa10016 | UBE2R2 | PRSS3 | Intra | Hiseq 2000 | 15 | | Chr9: 33807181-33910401 |
| BrCa10016 | PRKRIP1 | CUX1 | Intra | Hiseq 2000 | 10 | | Chr7: 101791060-101854134 |
| BrCa10016 | DLG1 | AK128161 | Inter | Hiseq 2000 | 9 | | Chr3: 198253827-198510540 |
| BrCa10017 | NFIX | MAST1 | Intra | HiSeq 2000 | 65 | Y | chr19: 12967584-13070610 |
| BrCa10017 | DYNLL1 | SMS | Inter | HiSeq 2000 | 48 | | chr12: 119418242-119420681 |
| BrCa10017 | RGS10 | ANTXRL | Intra | HiSeq 2000 | 47 | | chr10: 121249329-121292212 |
| BrCa10017 | PFKFB3 | RGS10 | Intra | HiSeq 2000 | 43 | | chr10: 6284901-6317501 |
| BrCa10017 | MCM7 | GPC2 | Intra | HiSeq 2000 | 25 | | chr7: 99528340-99537363 |
| BrCa10017 | DIP2C | LARGE | Inter | HiSeq 2000 | 15 | | chr10: 311432-725606 |
| BrCa10017 | EIF4G3 | C10ORF140 | Inter | HiSeq 2000 | 17 | | chr1: 21005561-21375927 |
| BrCa10017 | SMS | BX537644 | Inter | HiSeq 2000 | 10 | | chrX: 21868763-21922876 |
| BrCa10018 | SLC38A10 | BAIAP2 | Intra | Hiseq 2000 | 15 | | Chr17: 76833393-76883691 |
| BrCa10018 | MELK | RNF38 | Intra | Hiseq 2000 | 12 | | Chr9: 36562904-36667679 |
| BrCa10018 | CCNB1IP1 | PLEKHG1 | Inter | Hiseq 2000 | 11 | | Chr14: 19849368-19871297 |
| BrCa10018 | NKD2 | CCDC57 | Inter | Hiseq 2000 | 7 | | Chr5: 1062167-1091925 |
| BrCa10018 | SGPP2 | OPRL1 | Inter | Hiseq 2000 | 7 | | Chr2: 222997565-223131861 |
| BrCa10020 | PSME3 | CR597597 | Intra | Hiseq 2000 | 45 | | Chr17: 38229968-38249303 |
| BrCa10020 | NADSYN1 | SUV420H1 | Intra | Hiseq 2000 | 30 | | Chr11: 70841864-70890229 |
| BrCa10020 | SHANK2 | FGF3 | Intra | Hiseq 2000 | 4 | | Chr11: 69991608-70420323 |
| BrCa10021 | MAN2A2 | COL9A3 | Inter | Hiseq 2000 | 140 | | Chr15: 89247159-89266819 |
| BrCa10021 | PSKH1 | TSNAXIP1 | Intra | Hiseq 2000 | 101 | | Chr16: 66484715-66521082 |
| BrCa10021 | CSPP1 | BX248273 | Inter | Hiseq 2000 | 15 | | Chr8: 68139156-68271050 |
| BrCa10021 | NPAS3 | MIPOL1 | Intra | Hiseq 2000 | 10 | | Chr14: 32478209-33340702 |
| BrCa10025 | APBA1 | DEGS1 | Inter | Hiseq 2000 | 20 | | Chr9: 71235021-71477042 |
| BrCa10025 | AK096045 | CAPN5 | Intra | Hiseq 2000 | 11 | | Chr11: 70507522-70641187 |
| BrCa10025 | UVRAG | SLAMF1 | Inter | Hiseq 2000 | 7 | | Chr11: 75203859-75532930 |
| BrCa10025 | CBX5 | DCD | Intra | Hiseq 2000 | 6 | | Chr12: 52910996-52960182 |
| BrCa10025 | RFX3 | RB1 | Inter | Hiseq 2000 | 6 | | Chr9: 3214646-3515983 |
| BrCa10025 | BCL2L14 | ETV6 | Intra | Hiseq 2000 | 5 | | Chr12: 12115144-12255214 |
| BrCa10025 | DSE | FHL5 | Intra | Hiseq 2000 | 4 | | Chr6: 116707975-116866135 |
| BrCa10026 | TAOK1 | CCDC46 | Intra | Hiseq 2000 | 91 | | Chr17: 24742068-24895628 |
| BrCa10026 | MYO1D | FAM33A | Intra | Hiseq 2000 | 60 | | Chr17: 27843740-28228015 |
| BrCa10026 | MSI2 | NEK8 | Intra | Hiseq 2000 | 30 | | Chr17: 52688929-53112298 |
| BrCa10026 | GGNBP2 | C17ORF80 | Intra | Hiseq 2000 | 15 | | Chr17: 31974916-32020389 |
| BrCa10026 | BAZ1B | AUTS2 | Intra | Hiseq 2000 | 13 | | Chr7: 72492675-72574544 |
| BrCa10026 | RICS | LDLRAD3 | Intra | Hiseq 2000 | 11 | | Chr11: 128343051-128567303 |
| BrCa10026 | ST14 | ERN1 | Inter | Hiseq 2000 | 11 | | Chr11: 129534891-129585467 |
| BrCa10026 | KIAA0195 | MGAT5B | Intra | Hiseq 2000 | 10 | | Chr17: 70964258-71008128 |
| BrCa10027 | MPRIP | UBB | Intra | Hiseq 2000 | 49 | | Chr17: 16886831-17029598 |
| BrCa10027 | SMARCE1 | TMEM99 | Intra | Hiseq 2000 | 34 | | Chr17: 36037505-36057629 |
| BrCa10027 | TLK1 | C6ORF225 | Inter | Hiseq 2000 | 33 | | Chr2: 171556813-171796070 |
| BrCa10027 | FAM107B | FRMD4A | Intra | Hiseq 2000 | 32 | | Chr10: 14600564-14856902 |
| BrCa10027 | CALU | CAP2 | Inter | Hiseq 2000 | 31 | | Chr7: 128166671-128198764 |
| BrCa10027 | ADRBK2 | SGSM3 | Intra | Hiseq 2000 | 26 | | Chr22: 24290860-24455258 |
| BrCa10027 | DAPK3 | FERMT1 | Inter | Hiseq 2000 | 12 | | Chr19: 3909451-3922038 |
| BrCa10027 | MYH9 | MYO18B | Intra | Hiseq 2000 | 12 | | Chr22: 35007271-35113927 |
| BrCa10028 | RARA | KRT14 | Intra | Hiseq 2000 | 64 | | Chr17: 35718971-35820467 |
| BrCa10028 | ABL1 | EXOSC2 | Intra | Hiseq 2000 | 30 | | Chr9: 132579088-132752883 |
| BrCa10028 | AGPAT5 | MSRA | Intra | Hiseq 2000 | 25 | | Chr8: 6553285-6606429 |
| BrCa10028 | C9ORF25 | AGL | Inter | Hiseq 2000 | 14 | | chr9: 34388182-34448568 |
| BrCa10028 | CDKN2A | AX747623 | Intra | Hiseq 2000 | 11 | | Chr9: 21957750-21984490 |
| BrCa10028 | KCTD20 | SORBS2 | Inter | Hiseq 2000 | 7 | | Chr6: 36518521-36566293 |
| BrCa10029 | ZNF562 | RAD23A | Intra | Hiseq 2000 | 480 | | Chr19: 9620352-9646734 |
| BrCa10029 | JMJD2B | CAPS | Intra | Hiseq 2000 | 103 | | Chr19: 4920123-5104608 |
| BrCa10029 | ORC2L | SF3B1 | Intra | Hiseq 2000 | 41 | | Chr2: 201483138-201536655 |
| BrCa10029 | USP32 | LASS6 | Inter | Hiseq 2000 | 35 | | Chr17: 55609472-55824368 |
| BrCa10029 | ARFGEF2 | FAM65C | Intra | Hiseq 2000 | 28 | | Chr20: 46971681-47086637 |
| BrCa10029 | RNF43 | AKAP1 | Intra | Hiseq 2000 | 23 | | Chr17: 53786036-53849930 |
| BrCa10029 | SPAG9 | PHOSPHO1 | Intra | Hiseq 2000 | 17 | | Chr17: 46397986-46553094 |
| BrCa10029 | ITCH | ARFGEF2 | Intra | Hiseq 2000 | 16 | | Chr20: 32414722-32562858 |
| BrCa10029 | RAB22A | BSG | Inter | Hiseq 2000 | 11 | | Chr20: 56318176-56375969 |
| BrCa10029 | RAE1 | ZMYND8 | Intra | Hiseq 2000 | 9 | | Chr20: 55359551-55386926 |
| BrCa10029 | LATS1 | ESR1 | Intra | Hiseq 2000 | 7 | | Chr6: 150023743-150081085 |
| BrCa10029 | RAB6B | PDIA5 | Intra | Hiseq 2000 | 6 | | Chr3: 135025769-135097381 |
| BrCa10029 | PDIA5 | RAB6B | Intra | Hiseq 2000 | 4 | | Chr3: 124268654-124363565 |
| BrCa10030 | DNAJC3 | CLDN10 | Intra | Hiseq 2000 | 65 | | Chr13: 95127483-95241285 |
| BrCa10030 | KPNA2 | OSBPL9 | Inter | Hiseq 2000 | 56 | | Chr17: 63462309-63473432 |
| BrCa10030 | PRMT5 | HOMEZ | Intra | Hiseq 2000 | 30 | | Chr14: 22459572-22468501 |
| BrCa10030 | HIPK3 | NRD1 | Inter | Hiseq 2000 | 26 | | Chr11: 33235743-33332515 |
| BrCa10030 | CCDC47 | CACNB1 | Intra | Hiseq 2000 | 8 | | Chr17: 59176341-59204820 |
| BrCa10030 | HIVEP3 | SUPT6H | Inter | Hiseq 2000 | 8 | | Chr1: 41748270-42157083 |
| BrCa10030 | B4GALT1 | CSPP1 | Inter | Hiseq 2000 | 7 | | Chr9: 33100638-33157356 |
| BrCa10033 | ST3GAL3 | PTPRF | Intra | HiSeq 2000 | 72 | | chr1: 43,945,805-44,169,418 |
| BrCa10033 | TRAF4 | SPTAN1 | Inter | HiSeq 2000 | 22 | | chr17: 24,095,150-24,102,103 |
| BrCa10033 | MACF1 | RRAGC | Intra | HiSeq 2000 | 16 | | chr1: 39,569,397-39,725,376 |
| BrCa10033 | NOTCH1 | SNHG7 | Intra | HiSeq 2000 | 5 | Y | chr9: 138508717-138560059 |
| BrCa10035 | TSPAN15 | HK1 | Intra | HiSeq 2000 | 582 | | chr10: 70,881,232-70,937,429 |
| BrCa10035 | TMEM48 | SSBP3 | Intra | HiSeq 2000 | 91 | | chr1: 54,005,976-54,076,763 |
| BrCa10035 | MRPL27 | SPAG9 | Intra | HiSeq 2000 | 83 | | chr17: 45,800,227-45,805,561 |
| BrCa10035 | EHBP1 | COMMD1 | Intra | HiSeq 2000 | 63 | | chr2: 62,786,637-63,127,125 |
| BrCa10035 | RPS6KB1 | TMEM49 | Intra | HiSeq 2000 | 58 | | chr17: 55,325,225-55,382,568 |
| BrCa10035 | ZC3H7B | RER1 | Inter | HiSeq 2000 | 29 | | chr22: 40,027,513-40,086,097 |
| BrCa10035 | ADAM9 | PLEKHA2 | Intra | HiSeq 2000 | 25 | | chr8: 38,973,662-39,081,936 |
| BrCa10035 | RASGEF1A | HNRNPF | Intra | HiSeq 2000 | 22 | | chr10: 43,009,990-43,082,373 |
| BrCa10035 | ABHD12 | ZNF337 | Intra | HiSeq 2000 | 14 | | chr20: 25,223,379-25,319,477 |
| BrCa10036 | ANKS1B | CCDC53 | Intra | HiSeq 2000 | 13 | | chr12: 97,653,202-98,902,563 |
| BrCa10036 | RELB | CBLC | Intra | HiSeq 2000 | 13 | | chr19: 50,196,552-50,233,292 |
| BrCa10036 | PRMT2 | RCAN1 | Intra | HiSeq 2000 | 8 | | chr21: 46,879,955-46,909,291 |
| BrCa10037 | KDM4A | RASSF5 | Intra | HiSeq 2000 | 107 | | chr1: 43,888,384-43,943,776 |
| BrCa10037 | PRR12 | POLD1 | Intra | HiSeq 2000 | 49 | | chr19: 54,786,724-54,821,508 |
| BrCa10037 | RBM6 | BSN | Intra | HiSeq 2000 | 9 | | chr3: 49,952,596-50,089,686 |

| | | aCGH Data (5′ & 3′) | | | |
|---|---|---|---|---|---|
| Sample | Chromosomal Location | | Avg. log | | Avg log |
| Name | 3′ Gene | # Probe | ratio | # Probe | ratio |

Breast cell lines

| | | | | | |
|---|---|---|---|---|---|
| BT-20 | chr9: 100090187-100511300 | | | | |
| BT-20 | chr3: 123188859-123223720 | | | | |
| BT-20 | chr2: 131390693-131521306 | | | | |
| BT-474 | chr17: 44362457-44377153 | 5 | 2.890 | 2 | 3.557 |
| BT-474 | chr20: 55360024-55386926 | 4 | 2.910 | 4 | 2.910 |
| BT-474 | chr20: 33506636-33563217 | 15 | 3.650 | 5 | 1.876 |
| BT-474 | chr17: 33706516-33732628 | 11 | 3.290 | 4 | 3.452 |
| BT-474 | chr17: 50401124-50596448 | 4 | 4.029 | 21 | 2.507 |
| BT-474 | chr17: 32949013-33043559 | 1 | 2.787 | 10 | 2.556 |
| BT-474 | chr17: 34620314-34635566 | 35 | 2.556 | 3 | 4.029 |
| BT-474 | chr17: 56109953-56824981 | 13 | 1.012 | 73 | 1.934 |
| BT-474 | chr17: 35174724-35273967 | 7 | 3.404 | 10 | 3.701 |
| BT-474 | chr19: 17047590-17185104 | 6 | 3.404 | 13 | 2.122 |
| BT-474 | chr3: 32408166-32471337 | 11 | −0.425 | 6 | 0.428 |
| BT-474 | chr8: 81703240-81949571 | 35 | 0.916 | 26 | 0.640 |
| BT-474 | chr17: 57374747-57497425 | 73 | 1.934 | 13 | 1.012 |
| BT-474 | chr17: 54124961-54166691 | 6 | 4.813 | 5 | 1.700 |
| BT-474 | chr12: 84897167-85756812 | 19 | 1.218 | 90 | −0.397 |
| BT-474 | chr8: 25098203-25326536 | 5 | 4.821 | 27 | 0.076 |
| BT-474 | chr17: 31925711-31965418 | 14 | 2.244 | 6 | 2.344 |
| BT-483 | chr14: 102921453-103039919 | 8 | 1.170 | 17 | 0.381 |
| BT-549 | chr17: 55139644-55272734 | 9 | −0.283 | 18 | −1.185 |
| CAL-148 | chr1: 7,994,381-8,008,943 | | | | |
| CAL-148 | chr3: 49,736,732-49,798,977 | | | | |
| CAMA-1 | chr7: 150884133-150960277 | | | | |
| CAMA-1 | chr15: 43714547-43770771 | | | | |
| EFM-19 | chr16: 30,326,867-30,338,231 | | | | |
| EFM-19 | chr1: 77,934,262-77,998,125 | | | | |
| EFM-19 | chr8: 133,948,387-134,216,325 | | | | |
| EFM-19 | chr1: 52,380,634-52,584,946 | | | | |
| EFM-19 | chr9: 34,388,182-34,448,568 | | | | |
| EFM-19 | chr16: 51,646,446-51,918,915 | | | | |
| EFM-19 | chr10: 80,591,816-80,746,279 | | | | |
| HCC1008 | chr19: 12709337-12709449 | | | | |
| HCC1008 | chr22: 38468995-38619740 | | | | |
| HCC1008 | chr22: 27468043-27483496 | | | | |
| HCC1008 | chr3: 150720722-150858472 | | | | |
| HCC1008 | chr4: 2596957-2704100 | | | | |
| HCC1008 | chr4: 2815382-2901587 | | | | |
| HCC1008 | chr12: 112021701-112058404 | | | | |
| HCC1008 | chr10: 5444518-5490426 | | | | |
| HCC1008 | chr1: 113045272-113051201 | | | | |
| HCC1008 | chr11: 125678857-125720854 | | | | |
| HCC1008 | chr6: 4721682-4900777 | | | | |
| HCC1008 | chr16: 67328696-67426945 | | | | |
| HCC1008 | chr1: 206262211-206484288 | | | | |
| HCC1008 | chr2: 96354873-96357806 | | | | |
| HCC1008 | chr1: 8335051-8800286 | | | | |
| HCC1008 | chr2: 170149096-170202500 | | | | |
| HCC1008 | chr8: 80685935-80739781 | | | | |
| HCC1008 | chr20: 2465253-2570430 | | | | |
| HCC1008 | chr22: 23796180-23920764 | | | | |
| HCC1008 | chr1: 8335051-8800286 | | | | |
| HCC1143 | chr20: 29565901-29591257 | 18 | 1.280 | 2 | 1.403 |
| HCC1143 | chr2: 10180145-10188997 | 8 | 0.134 | 2 | 0.134 |
| HCC1187 | chr6: 42300646-42527761 | 14 | 1.648 | 27 | 0.336 |
| HCC1187 | chr1: 120255698-120413799 | 2 | 1.557 | 11 | 0.253 |
| HCC1187 | chr14: 38653238-38675928 | 9 | 0.940 | 4 | 0.235 |
| HCC1187 | chr8: 6553285-6606429 | 29 | 0.495 | 5 | 0.738 |
| HCC1187 | chr10: 73225533-73245710 | 3 | 0.888 | 1 | 0.953 |
| HCC1187 | chr1: 11049262-11082525 | 1 | 0.816 | 4 | 0.156 |
| HCC1395 | chr6: 46625403-46728482 | 2 | 0.852 | 11 | 0.611 |
| HCC1395 | chr11: 62039949-62070908 | 2 | 0.629 | 5 | 1.172 |
| HCC1395 | chr3: 199002541-199082853 | 10 | 0.755 | 11 | −0.615 |
| HCC1395 | chr14: 52395955-52487565 | 7 | 0.934 | 14 | 0.934 |
| HCC1395 | chr2: 27966985-28415271 | 3 | 0.480 | 51 | 0.480 |
| HCC1395 | chr1: 94230981-94359293 | 13 | 0.849 | 13 | 0.849 |
| HCC1419 | chr8: 96106396-96140114 | | | | |
| HCC1419 | chr15: 38886566-38894059 | | | | |
| HCC1419 | chr8: 87553694-87590125 | | | | |
| HCC1419 | chr17: 45270669-45280378 | | | | |
| HCC1419 | chr9: 130111216-130124518 | | | | |
| HCC1419 | chr20: 48636053-48686833 | | | | |
| HCC1419 | chr20: 51235228-51545276 | | | | |
| HCC1419 | chr8: 145624971-145640620 | | | | |
| HCC1428 | chr17: 44927654-44947371 | | | | |
| HCC1428 | chr21: 42512336-42590423 | | | | |
| HCC1428 | chr6: 151856920-151984021 | | | | |
| HCC1428 | chr1: 226462483-226633198 | | | | |
| HCC1428 | chr9: 122402912-122516433 | | | | |
| HCC1428 | chr1: 70944724-71024737 | | | | |
| HCC1428 | chr1: 64012278-64417295 | | | | |
| HCC1428 | chr1: 29011241-29062795 | | | | |
| HCC1500 | chr5: 125906817-125958981 | | | | |
| HCC1500 | chr20: 32045393-32131728 | | | | |
| HCC1569 | chr11: 58050919-58099966 | | | | |
| HCC1569 | chr16: 21872109-21902169 | | | | |
| HCC1569 | chr6: 1569039-2190845 | | | | |
| HCC1569 | chr11: 33018136-33051685 | | | | |
| HCC1569 | chr17: 61062119-61618449 | | | | |
| HCC1569 | chr17: 35046858-35073980 | | | | |
| HCC1569 | chr8: 19341621-19504550 | | | | |
| HCC1599 | chr17: 74181724-74289971 | | | | |
| HCC1599 | chr17: 71818462-71861825 | | | | |
| HCC1599 | chr3: 187121586-187138450 | | | | |
| HCC1599 | chr4: 56899213-56948395 | | | | |
| HCC1599 | chr3: 158026841-158125739 | | | | |
| HCC1806 | chr11: 64705918-64736052 | | | | |
| HCC1806 | chr20: 32331731-32363269 | | | | |
| HCC1806 | chr2: 180517848-180580025 | | | | |
| HCC1806 | chr16: 22980228-23068092 | | | | |
| HCC1937 | chr11: 34599244-34639657 | | | | |
| HCC1937 | chr12: 44599181-44670668 | | | | |
| HCC1954 | chr6: 34613557-34632069 | 13 | 0.036 | 3 | 0.374 |
| HCC1954 | chr7: 555359-718687 | 4 | 1.034 | | |
| HCC1954 | chr2: 148408201-148494933 | 15 | 0.409 | 7 | 0.504 |
| HCC202 | chr17: 44362457-44377153 | | | | |
| HCC2157 | chr17: 35036705-35046404 | | | | |
| HCC2157 | chr9: 133154945-133174468 | | | | |
| HCC2157 | chr1: 245266710-245308692 | | | | |
| HCC2157 | chr11: 65594400-65768789 | | | | |
| HCC2157 | chr1: 36169359-36294650 | | | | |
| HCC2157 | chr6: 36272528-36308545 | | | | |
| HCC2157 | chr3: 142433372-142496176 | | | | |
| HCC2157 | chr22: 18388631-18433447 | | | | |
| HCC2157 | chr22: 18388631-18433447 | | | | |
| HCC2157 | chr10: 73797104-74055905 | | | | |
| HCC2157 | chr1: 12212700-12494685 | | | | |
| HCC2157 | chr19: 10457796-10475054 | | | | |
| HCC2157 | chr16: 66855924-66893223 | | | | |
| HCC2157 | chr2: 29057668-29128600 | | | | |
| HCC2157 | chr9: 114953059-114966243 | | | | |
| HCC2157 | chr18: 72200607-72304085 | | | | |
| HCC2157 | chr4: 48582162-48603572 | | | | |
| HCC2157 | chr14: 34100369-34169117 | | | | |
| HCC2218 | chr9: 138508716-138560059 | 6 | 0.000 | 7 | −0.967 |
| HCC2218 | chr17: 57111328-57295702 | 3 | 1.113 | 19 | 3.925 |
| HCC2218 | chr17: 44721566-44794834 | 9 | 3.925 | 6 | 2.649 |
| HCC2218 | chr17: 55139644-55272734 | 9 | 3.925 | 18 | 3.202 |
| HCC2218 | chr17: 35013546-35017701 | 3 | 2.649 | 1 | 3.451 |
| HCC2218 | chr17: 56032335-56096818 | 2 | 3.451 | 7 | 3.340 |
| HCC38 | chr11: 101952776-102001273 | | | | |
| HCC38 | chr4: 30331135-30757519 | | | | |
| HCC38 | chr5: 149090057-149207462 | | | | |
| HCC38 | chr15: 54998125-55368006 | | | | |
| HCC38 | chr17: 35314374-35328429 | | | | |
| HCC38 | chr15: 38240501-38300629 | | | | |
| HCC38 | chr1: 157246306-157291569 | | | | |
| HCC38 | chr2: 45732546-46268633 | | | | |
| HCC38 | chr1: 143807764-143828279 | | | | |
| HCC38 | chr11: 123100207-123117573 | | | | |
| HCC38 | chr1: 234916393-234994181 | | | | |
| HCC38 | chr1: 216525252-216577948 | | | | |
| HCC38 | chr15: 81567384-81596680 | | | | |
| HCC38 | chr14: 22564900-22573949 | | | | |
| HCC38 | chr19: 54872307-54883516 | | | | |
| HCC38 | chr5: 108698309-108773574 | | | | |
| HCC70 | chr5: 364342-365660 | | | | |
| HCC70 | chr7: 5533305-5536758 | | | | |
| Hs578T | chr1: 154830903-154837894 | | | | |
| MCF7 | chr17: 56109953-56824981 | 7 | 2.107 | 73 | 2.653 |
| MCF7 | chr20: 45719556-45848215 | 11 | 0.823 | 13 | 3.398 |
| MCF7 | chr17: 55139644-55272734 | 5 | 3.412 | 18 | 2.197 |
| MCF7 | chr19: 1199551-1210142 | 4 | −1.367 | 2 | −0.279 |
| MCF7 | chr14: 95928200-96024865 | 7 | 0.343 | 13 | 0.343 |
| MCF7 | chr17: 54124961-54166691 | 4 | −0.063 | 5 | 2.788 |
| MCF7 | chr20: 48636052-48686833 | 12 | 0.456 | 5 | 1.554 |
| MCF7 | chr17: 55139644-55272734 | 1 | 3.515 | 18 | 2.197 |
| MCF7 | chr5: 128329108-128397234 | 30 | 0.051 | 8 | 0.051 |
| MCF7 | chr22: 30125538-30160172 | 8 | 0.387 | 5 | −0.420 |
| MCF7 | chr19: 17719526-17760377 | 13 | −1.126 | 4 | −0.529 |
| MCF7 | chr2: 198089016-198125760 | 1 | −0.361 | 4 | −0.361 |
| MCF7 | chr18: 13601664-13642753 | 10 | −0.674 | 5 | −0.407 |
| MCF7 | chr17: 55139644-55272734 | 14 | 3.515 | 18 | 2.197 |
| MCF7 | chr19: 10843252-10894448 | 8 | 0.041 | 6 | 0.041 |
| MCF7 | chr20: 45271787-45418881 | 7 | 2.107 | 15 | 3.860 |
| MCF7 | chr8: 128817496-128822862 | 27 | 1.186 | 3 | 1.186 |
| MCF7 | chr17: 55384504-55396899 | 14 | 3.515 | 2 | 3.412 |
| MDA-MB-134 | chr8: 40507267-40874500 | | | | |
| MDA-MB-134 | chr13: 112670757-112800863 | | | | |
| MDA-MB-157 | chr19: 52572006-52577795 | | | | |
| MDA-MB-157 | chr15: 39496593-39563053 | | | | |
| MDA-MB-157 | chr15: 23474952-23659442 | | | | |
| MDA-MB-157 | chr11: 75106581-75119979 | | | | |
| MDA-MB-175VII | chr11: 78041975-78829343 | | | | |
| MDA-MB-330 | chr17: 33046525-33077600 | | | | |
| MDA-MB-361 | chr17: 34871265-34944326 | 9 | 2.327 | 7 | 1.529 |
| MDA-MB-361 | chr17: 53921891-53950250 | 27 | 0.000 | 6 | 1.658 |
| MDA-MB-361 | chr16: 54782751-54939612 | 10 | −0.157 | 19 | 0.281 |
| MDA-MB-361 | chr17: 61062119-61618449 | | | | |
| MDA-MB-415 | chr11: 68572926-68614648 | | | | |
| MDA-MB-415 | chr11: 69991609-70185520 | | | | |
| MDA-MB-415 | chr11: 63509901-63522468 | | | | |
| MDA-MB-415 | chr11: 69602294-69713282 | | | | |
| MDA-MB-453 | chrX: 154375389-154495816 | 8 | 1.611 | 11 | 1.602 |
| MDA-MB-453 | chr17: 59053532-59127402 | 3 | 0.543 | 10 | 0.494 |
| MDA-MB-468 | chr8: 104480041-104496644 | 18 | 0.070 | 4 | 0.927 |
| MDA-MB-468 | chr1: 46041871-46274383 | 10 | 0.266 | 23 | 0.818 |
| MDA-MB-468 | chr19: 55579404-55613083 | 17 | 4.944 | 4 | 0.732 |
| MDA-MB-468 | chr11: 33724866-33752647 | 2 | 0.853 | 3 | 1.507 |
| SUM149PT | chr10: 99614747-99780575 | | | | |
| SUM149PT | chr2: 120487254-120580962 | | | | |
| SUM190PT | chr17: 27682501-27693302 | | | | |
| SUM190PT | chr1: 188333419-188713382 | | | | |
| SUM190PT | chr16: 56230707-56256445 | | | | |
| SUM190PT | chr22: 19601713-19638037 | | | | |
| SUM190PT | chr17: 35082579-35097833 | | | | |
| SUM190PT | chr11: 72794675-72986876 | | | | |
| SUM190PT | chr2: 149349288-149591519 | | | | |
| SUM190PT | chr11: 73401413-73474579 | | | | |
| SUM190PT | chr4: 55164137-55168055 | | | | |
| SUM190PT | chr4: 109072165-109094062 | | | | |
| SUM190PT | chr1: 144167592-144181744 | | | | |
| T-47D | chr16: 65620551-65692459 | | | | |
| T-47D | chr3: 15271361-15349108 | | | | |
| T-47D | chr1: 17121032-17172061 | | | | |
| UACC-812 | chr1: 150222009-150233338 | | | | |
| UACC-812 | chr1: 149851285-149938183 | | | | |
| UACC-812 | chr17: 35109780-35138441 | | | | |
| UACC-812 | chr17: 35174724-35273967 | | | | |
| UACC-812 | chr17: 34473082-34563011 | | | | |
| UACC-893 | chr17: 34871265-34944326 | 17 | 2.069 | 7 | 4.175 |
| UACC-893 | chr10: 61458164-61570752 | 17 | 0.890 | 13 | 0.890 |
| UACC-893 | chr17: 35038278-35046404 | 1 | 4.843 | 2 | 4.843 |
| UACC-893 | chr17: 35174724-35273967 | 4 | 3.908 | 10 | 4.843 |
| UACC-893 | chr2: 37331149-37397726 | 8 | 1.213 | 8 | 1.278 |
| ZR-75-1 | chr1: 6767970-6854694 | 17 | −0.380 | 10 | −0.089 |
| ZR-75-1 | chr1: 6767970-6854694 | 3 | −0.225 | 10 | −0.089 |
| ZR-75-1 | chr1: 17605837-17637644 | 4 | −0.013 | 4 | −0.225 |
| ZR-75-30 | chr17: 34210972-34235115 | | | | |
| ZR-75-30 | chr8: 120955080-121132338 | | | | |
| ZR-75-30 | chr8: 120638499-120720287 | | | | |
| ZR-75-30 | chr17: 44053517-44058834 | | | | |
| ZR-75-30 | chr17: 34143675-34158084 | | | | |
| ZR-75-30 | chr17: 56109953-56824981 | | | | |

Breast tumor tissues

| | |
|---|---|
| BrCa00001 | chr6: 151856867-151984021 |
| BrCa00001 | chr19: 12810258-12846766 |
| BrCa00001 | chr7: 98830524-98844228 |
| BrCa00002 | chr19: 19533521-19590439 |
| BrCa00002 | chr17: 54587641-54638852 |
| BrCa00003 | chr17: 46609784-46692426 |
| BrCa00004 | chr10: 135128246-135187052 |
| BrCa00005 | chr16: 3002457-3004507 |
| BrCa00006 | chr8: 104900591-105334627 |
| BrCa00007 | chr17: 33276685-33318476 |
| BrCa00007 | chr8: 99145926-99175014 |
| BrCa10001 | Chr18: 54489597-54568350 |
| BrCa10001 | Chr1: 239005439-239587101 |
| BrCa10001 | Chr2: 241301856-241408297 |
| BrCa10002 | chr9: 138722683-138722837 |
| BrCa10002 | Chr9: 831689-959090 |
| BrCa10003 | Chr22: 36945243-36998962 |
| BrCa10005 | Chr20: 56318176-56375969 |
| BrCa10005 | chr20: 60418688-60435984 |
| BrCa10005 | Chr8: 124933407-125201483 |
| BrCa10005 | Chr3: 62359060-62836094 |
| BrCa10005 | Chr16: 20699015-20819161 |
| BrCa10005 | Chr11: 62432726-62445588 |
| BrCa10006 | Chr16: 56103590-56127978 |
| BrCa10006 | Chr17: 57114766-57295537 |
| BrCa10007 | Chr10: 69535881-69641779 |
| BrCa10007 | Chr10: 131523536-131652081 |
| BrCa10007 | Chr12: 110282865-110291308 |
| BrCa10007 | Chr11: 832823-857116 |
| BrCa10008 | Chr8: 126173276-126448544 |
| BrCa10008 | Chr1: 149603403-149611788 |
| BrCa10008 | Chr8: 49036046-49052621 |
| BrCa10008 | Chr7: 141054621-141077540 |
| BrCa10008 | Chr10: 75074649-75085838 |
| BrCa10008 | ChrX: 47316243-47364200 |
| BrCa10008 | Chr12: 970664-1472958 |
| BrCa10008 | Chr16: 28472757-28510610 |
| BrCa10008 | Chr11: 63030131-63040815 |
| BrCa10008 | chr16: 631850-638475 |
| BrCa10008 | Chr1: 226937491-226949034 |
| BrCa10009 | chr3: 127863614-127873472 |
| BrCa10009 | Chr1: 45749293-45760196 |
| BrCa10009 | Chr12: 120761815-120810900 |
| BrCa10009 | Chr15: 61356843-61385166 |
| BrCa10010 | Chr8: 75890840-75941729 |
| BrCa10011 | Chr8: 103633035-103642422 |
| BrCa10011 | chr21: 18083155-18113574 |
| BrCa10011 | Chr20: 31459423-31495359 |
| BrCa10011 | Chr5: 174838188-174888218 |
| BrCa10011 | chr4: 20,311,179-20,335,609 |
| BrCa10011 | Chr11: 67984774-68139377 |
| BrCa10011 | Chr8: 101232014-101235406 |
| BrCa10014 | Chr11: 63758841-63762835 |
| BrCa10015 | Chr12: 68028400-68034280 |
| BrCa10015 | Chr14: 95412905-95461652 |
| BrCa10016 | Chr14: 23857409-23874117 |
| BrCa10016 | Chr9: 33740514-33789229 |
| BrCa10016 | Chr7: 101246011-101713970 |
| BrCa10016 | Chr8: 93794365-93959041 |
| BrCa10017 | chr19: 12810348-12846765 |
| BrCa10017 | chrX: 21868763-21922876 |
| BrCa10017 | chr10: 47128240-47171452 |
| BrCa10017 | chr10: 121249329-121292212 |
| BrCa10017 | chr7: 99605165-99612926 |
| BrCa10017 | chr22: 31999062-32646416 |
| BrCa10017 | chr10: 21842415-21854617 |
| BrCa10017 | chr12: 119413206-119418111 |
| BrCa10018 | Chr17: 76623556-76705827 |
| BrCa10018 | Chr9: 36326398-36391195 |
| BrCa10018 | Chr6: 150962691-151206492 |
| BrCa10018 | Chr17: 77652634-77763978 |
| BrCa10018 | Chr20: 62181931-62202440 |
| BrCa10020 | Chr17: 16662065-16666618 |
| BrCa10020 | Chr11: 67680082-67737681 |
| BrCa10020 | Chr11: 69333916-69343129 |
| BrCa10021 | Chr20: 60918858-60942956 |
| BrCa10021 | Chr16: 66398316-66419472 |
| BrCa10021 | Chr14: 40493665-40514820 |
| BrCa10021 | Chr14: 36736906-37090215 |
| BrCa10025 | Chr1: 222430080-222447765 |
| BrCa10025 | Chr11: 76455639-76514846 |
| BrCa10025 | Chr1: 158846514-158883705 |
| BrCa10025 | Chr12: 53324641-53328416 |
| BrCa10025 | Chr13: 47775883-47954027 |
| BrCa10025 | Chr12: 11694054-12159528 |
| BrCa10025 | Chr6: 97117155-97171233 |
| BrCa10026 | Chr17: 61062119-61618674 |
| BrCa10026 | Chr17: 54542089-54587582 |
| BrCa10026 | Chr17: 24079958-24093911 |
| BrCa10026 | chr17: 68740371-68756690 |
| BrCa10026 | Chr7: 68701840-69895821 |
| BrCa10026 | Chr11: 35922187-36209417 |
| BrCa10026 | Chr17: 59474121-59561234 |
| BrCa10026 | Chr17: 72376392-72458066 |
| BrCa10027 | Chr17: 16225091-16226779 |
| BrCa10027 | Chr17: 36228899-36246052 |
| BrCa10027 | chr6: 112515367-112530686 |
| BrCa10027 | Chr10: 13725711-14412872 |
| BrCa10027 | Chr6: 17501714-17666002 |
| BrCa10027 | Chr22: 39096540-39136239 |
| BrCa10027 | Chr20: 6003491-6052191 |
| BrCa10027 | Chr22: 24468119-24757007 |
| BrCa10028 | Chr17: 36992058-36996673 |
| BrCa10028 | Chr9: 132558978-132570273 |
| BrCa10028 | Chr8: 9949235-10323805 |
| BrCa10028 | Chr1: 100088227-100162167 |
| BrCa10028 | Chr9: 22636198-22814212 |
| BrCa10028 | Chr4: 186743591-187114516 |
| BrCa10029 | Chr19: 12917653-12925455 |
| BrCa10029 | Chr19: 5865218-5866886 |
| BrCa10029 | Chr2: 197964942-198008016 |
| BrCa10029 | Chr2: 169021080-169339398 |
| BrCa10029 | Chr20: 48636052-48686833 |
| BrCa10029 | Chr17: 52517551-52553709 |
| BrCa10029 | Chr17: 44655730-44663127 |
| BrCa10029 | Chr20: 46971681-47086637 |
| BrCa10029 | Chr19: 522324-534493 |
| BrCa10029 | Chr20: 45271787-45418881 |
| BrCa10029 | Chr6: 152053323-152466101 |
| BrCa10029 | Chr3: 124268654-124363565 |
| BrCa10029 | Chr3: 135025769-135097381 |
| BrCa10030 | Chr13: 94883858-95029907 |
| BrCa10030 | Chr1: 51815438-52026726 |
| BrCa10030 | Chr14: 22812683-22838590 |
| BrCa10030 | Chr1: 52027453-52117197 |
| BrCa10030 | Chr17: 34583234-34607427 |
| BrCa10030 | Chr17: 24013428-24053376 |
| BrCa10030 | Chr8: 68139156-68271050 |
| BrCa10033 | chr1: 43,769,134-43,861,930 |
| BrCa10033 | chr9: 130,354,687-130,435,761 |
| BrCa10033 | chr1: 39,077,606-39,097,927 |
| BrCa10033 | chr9: 138735639-138742457 |
| BrCa10035 | chr10: 70,748,609-70,831,643 |
| BrCa10035 | chr1: 54,464,783-54,644,680 |
| BrCa10035 | chr17: 46,397,987-46,553,094 |
| BrCa10035 | chr2: 61,986,307-62,216,709 |
| BrCa10035 | chr17: 55,139,645-55,272,734 |
| BrCa10035 | chr1: 2,313,074-2,326,734 |
| BrCa10035 | chr8: 38,877,910-38,950,587 |
| BrCa10035 | chr10: 43,201,071-43,223,305 |
| BrCa10035 | chr20: 25,602,851-25,625,469 |
| BrCa10036 | chr12: 100,930,848-100,980,029 |
| BrCa10036 | hr19: 49,972,988-49,995,731 |
| BrCa10036 | chr21: 34,810,654-34,909,252 |
| BrCa10037 | chr1: 204,747,502-204,829,239 |
| BrCa10037 | chr19: 55,579,405-55,613,083 |
| BrCa10037 | chr3: 49,566,926-49,683,986 |

TABLE 5

| Sample | 5′ Gene | 3′ Gene | Type |
|---|---|---|---|
| MDA-MB-453 | MYO15B | MAP3K3 | Kinase |
| BrCa10026 | MSI2 | NEKB | Kinase |
| HCC38 | SPRED1 | BUB1B | Kinase |
| BrCa00006 | STK3 | RIMS2 | Kinase |
| HCC1954 | INTS1 | PRKAR1B | Kinase Regulatory |
| HCC1569 | PTPRJ | LPXN | Phosphatase |
| BrCa10025 | BCL2L14 | ETV6 | Transcription Factor |
| BrCa10035 | RELB | CBLC | Transcription Factor/ |
| | | | Oncogene |
| ZR-75-1 | FOXJ3 | CAMTA1 | Transcription Factor |
| HCC1419 | VAV2 | TRUB2 | Oncogene |
| BrCa10001 | SEC11C | MALT1 | Oncogene |
| SUM190PT | KLHL22 | CRKL | Oncogene |
| BrCa10021 | NPAS3 | MIPOL1 | Tumor Supressor |
| BrCa10025 | RFX3 | RB1 | Tumor Supressor |
| BrCa10037 | KDM4A | RASSF5 | Tumor Supressor |
| BrCa10014 | MACROD1 | VEGFB | Ligand |
| BrCa10006 | GPATCH8 | BRIP1 | BRCA1 Interacter |
| BfCa10035 | RASGEF1A | HNRNPF | GEF |

TABLE 6

| | | SEQ |
|---|---|---|
| | | ID |
| | | NO.: |
| Validation of MAST fusion candidates | | |
| TADA2A-MAST1-S1 | CCTGGCACAGAGAAGCTGAATGA | 1 |
| TADA2A-MAST1-AS1 | CAGGGCGTGAGATGATAATAAGCAA | 2 |
| TADA2A-MAST1-S2 | CCTGGCACAGAGAAGCTGAATGAAA | 3 |
| TADA2A-MAST1-AS2 | CTCGCAGGGCGTGAGATGATAA | 4 |
| NFIX_MAST1 S1 | TGTGCGTCCAGCCACATCACATTG | 5 |
| NFIX_MAST1 AS1 | TACTGGGGTCTTGGGCTCGTGCTG | 6 |
| NFIX_MAST1 S2 | CAGCCACATCACATTGGAGTCACAATC | 7 |
| NFIX_MAST1 AS2 | AGCTGCTACTGGGGTCTTGGGCTC | 8 |
| ARID1A-MAST2_f1 | GAGCCACCACGCGCCCAT | 9 |
| ARID1A-MAST2_r1 | CCTGAAGAGCAGGGGACTAACTCCA | 10 |
| ARID1A-MAST2_f2 | CATGAGCCCCGGGAGCAGC | 11 |
| ARID1A-MAST2_r2 | CCTGAAGAGCAGGGGACTAACTCCA | 12 |
| ARID1A-MAST2_Uf1 | CCAACAAAGGAGCCACCA | 13 |
| ARID1A-MAST2_Ur1 | GGACTAACTCCAGTTACTACATCCTGA | 14 |
| ZNF700-MAST1-f1 | CCCGGTACATCTGAAAGCCGGGA | 15 |
| ZNF700-MAST1-r1 | ACTGGCGGATTTCCACGGGC | 16 |
| ANF700-MAST1-f2 | TCTGTCGCTCTGTCGCCTGC | 17 |
| ZNF700-MAST1-r2 | TGGTGATACCTGTCTGAGCGGG | 18 |
| MAST1 and MAST2 target capture | | |
| T7 MAST1-S1 | GGATAATACGACTCACTATAGGGCCTCATCCTGACCAGCACTTCA | 19 |
| T7 MAST-AS1 | TTCGGGAGGAGGCAAACGAG | 20 |
| T7 MAST1-S2 | GGATAATACGACTCACTATAGGGCCTCGCTCCCTTCATCTGG | 21 |
| T7 MAST1-AS2 | TCGTCCACCGTGGGCTGGTA | 22 |
| T7 MAST1-S3 | GGATAATACGACTCACTATAGGGACAACGAGATCGTGATGATGAATC | 23 |
| T7 MAST1-AS3 | CAGAACGCTGTCGGGTTCGTA | 24 |
| T7 MAST1-S4 | GGATAATACGACTCACTATAGGGTACGAACCCGACAGCGTTCTG | 25 |
| T7 MAST1-AS4 | TGCAATTCATAGAAGTAGACCGTGG | 26 |
| T7 MAST1-S5 | GGATAATACGACTCACTATAGGGCCTATGAACGCTCTGAGAGCTTG | 27 |
| T7 MAST1-AS5 | TCCAGCAGGTGGTAGAACTCCTC | 28 |
| T7 MAST1-S6 | GGATAATACGACTCACTATAGGGTCAACCCCGAGGAGTTCTACCA | 29 |
| T7 MAST1-AS6 | ATGCACCACATCTGGAAAGGG | 30 |
| T7 MAST1-S7 | GGATAATACGACTCACTATAGGGTGCATCTGGAGGAACAGGA | 31 |
| T7 MAST1-AS7 | CGTTGCTTATGAGCTTGATGGTATC | 32 |
| T7 MAST1-S8 | GGATAATACGACTCACTATAGGGATACCATCAAGCTCATAAGCAACG | 33 |
| T7 MAST1-AS8 | TTGCGGAGGATCAAGTTCTGC | 34 |
| T7 MAST2-S1 | GGATAATACGACTCACTATAGGGTAACTGGAGTTAGTCCCCTGCTCTT | 35 |
| T7 MAST2-AS1 | CCAGGTTTCCTCTCCATAACTTACAA | 36 |
| T7 MAST-S2 | GGATAATACGACTCACTATAGGGTGCTCCCTTTGTCCAGCAGTGTA | 37 |
| T7 MAST-AS2 | CCAGCAGTAAGAGAAGGTGCAGAC | 38 |
| T7 MAST-S3 | GGATAATACGACTCACTATAGGGTTGAGCCTTCCAAGAAGAGGC | 39 |
| T7 MAST2-AS3 | TGTGGCCATGGAGTGGTGAG | 40 |
| T7 MAST2-S4 | GGATAATACGACTCACTATAGGGTCCAAATGCACCTGCTCACTTT | 41 |
| T7 MAST2-AS4 | CAGTGGAGCTAGGAGTGTTAGTTCCA | 42 |
| T7 MAST2-S5 | GGATAATACGACTCACTATAGGGAAAAGCTGCATCAGTTGCCT | 43 |
| T7 MAST2-AS5 | GGACTGCCGTCCTTCCTCATCT | 44 |
| T7 MAST2-S6 | GGATAATACGACTCACTATAGGGACGATCCCCAGTATCCTTTGA | 45 |
| T7 MAST2-AS6 | CGCTGTCTGGAGTGTTGGAGGAA | 46 |
| T7 MAST2-S7 | GGATAATACGACTCACTATAGGGCGACTAGCAGAGTTTATTTCCTCC | 47 |
| T7 MAST2-AS7 | CTCCGAGATTTATCCAGGCAGTC | 48 |
| T7 MAST2-S8 | GGATAATACGACTCACTATAGGGCTCAGAAGTGGCTTTTGTGATGC | 49 |
| T7 MAST2-AS8 | AAGGTGGTAGAACTCTTCAGGGTCA | 50 |
| T7 MAST2-S9 | GGATAATACGACTCACTATAGGGAATGCCTGGAGTTTGACCC | 51 |
| T7 MAST2-AS9 | AGCTGGCTAACGATGTAGCGG | 52 |
| T7 MAST2-S10 | GGATAATACGACTCACTATAGGGACAGTCCTGACACTCCAGAGACAGA | 53 |
| T7 MAST2-AS10 | CACCAGAAATACAGCCCCATAGG | 54 |
| MAST fusion constructs | | |
| NFIX-S | CAAACCATGGGGAGCGGCTCTACAAGTC | 55 |
| NFIX-MAST1 JUNC-S | TGGCTTACTTTGTCCACACTCCGGGTGTATAGCAGCATGGAGCAGC | 56 |
| NFIX-MAST1 JUNC-AS | CTGCTCCATGCTGCTATACACCCGGAGTGTGGACAAAGTAAGCCA | 57 |
| TADA2A-S | CAAACCATGGACCGTTTGGGTCCCTTTAG | 58 |
| TADA2A-MAST1 JUNC-S | GAAGCTGAATGAAAAAGAAAAGGAGGCCTATGAACGCTCTGAGAGCTT | 59 |
| TADA-MAST1 JUNC-AS | AAGCTCTCAGAGCGTTCATAGGCCTCCTTTTCTTTTTCATTCAGCTTC | 60 |
| ZNF MAST1-S1 | GAAACCATGTCAGGGGATGTGGCAGTAGA | 61 |
| ZNF MAST1-AS1 | GAACAGCACGGACGCACTTTAT | 62 |
| ZNF MAST1-AS2 | GAATTTTCACGCAGCACGGACGC | 63 |
| MAST1-AS1 | GAACACGGACGCACTTTATTTATATGT | 64 |
| MAST1-AS1 | GAACGGACCGTTCACGCAGCACGGACGCAC | 65 |
| MAST2-AS1 | CAACGGACCGCAGCACGGACGCACTTTATTTA | 66 |
| MAST2-AS2 | CAACGGACCGCACGCAGCACGGACGCACTTTAT | 67 |
| GPBP1L1-S1 | GAAACCATGCGGCCTCGCTCCCGGA | 68 |
| GPBP1L1-MAST2-AS1 | GAAGTCTGAGTGCAAGAAATGGCAAAC | 69 |
| GPBP1L1-MAST2-AS2 | GAACAAGAAATGGCAAACAACTGC | 70 |
| ARID1-S1 | CAAACCATGGCCGCGCAGGTCGCC | 71 |
| ARID-MAST2 JUNC-S | GCTCGCCCGGACCCCTCAGGATGTAGTAACTGGAGTTAGTCC | 72 |
| ARID-MAST2 JUNC-AS | GGACTAACTCCAGTTACTACATCCTGAGGGGTCCGGGCGAGC | 73 |
| Detection of NOTCH genomic fusion junctions | | |
| HCC1599 GENOMIC-F1 | ATCCAGGTGCTGCTGAGTCCA | 74 |
| HCC1599 GENOMIC-R1 | ATCCAGGTGCTGCTGAGTCCACT | 75 |
| HCC1599 GENOMIC-F2 | TGTCATCTGTGTCATCCACCCTG | 76 |
| HCC1599 GENOMIC-R2 | ATCCAGGTGCTGCTGAGTCCA | 77 |
| HCC2218 GENOMIC-F1 | TGTAGACAAGAGGCAAAATAGCGTG | 78 |
| HCC2218 GENOMIC-R1 | CGCCACGTACATGAAGTGCAG | 79 |
| HCC2218 GENOMIC-F2 | CAAGAGGCAAAATAGCGTGTCTTTC | 80 |
| HCC2218 GENOMIC-R2 | CCACGAAGAACAGAAGCACAAAGG | 81 |
| HCC1187 GENOMIC-F1 | GCTGCCATATTACCGAAGATGGAC | 82 |
| HCC1187 GENOMIC-R1 | ATTCCCACATAGAGGATGTCCCA | 83 |
| HCC1187 GENOMIC-F2 | TGCGGTTGTGTGTCAAGTTACTACC | 84 |
| HCC1187 GENOMIC-R2 | CCTTCCAGACATTCTGCCTCCTG | 85 |
| HCC1187 GENOMIC-F3 | GCTAACTGAACCAGCATGGTAAGGT | 86 |
| HCC1187 GENOMIC-R3 | GACATTCTGCCTCCTGTGTACCC | 87 |
| Validation of NOTCH fusion candidates | | |
| NOTCH1-del F1 | TGAGACCTGCCTGAATGGCGGGAA | 88 |
| NOTCH1-del R1 | GCCCACGAAGAACAGAAGCACAAAGG | 89 |
| SEC16A-NOTCH1 F1 | ACCCGAGCCGGATGTGCCAAGAT | 90 |
| SEC16A-NOTCH1 R1 | GCCGCCACGTACATGAAGTGCAG | 91 |
| SEC22B-NOTCH2 F1 | GATGGTGTTGCTAACAATGATCGC | 92 |
| SEC22B-NOTCH2 R1 | TGCATCCGTGTTCTTGAAGCAG | 93 |
| NOTCH1-SNHG7 F1 | CCTGAATGGCGGGAAGTGTGAAGC | 94 |
| NOTCH1-SNHG7 R1 | CTGCAAACACCCTGAGTGCCAGTG | 95 |
| NOTCH1-chr9 F1 | TCACCCACGAGTGTGCCTGCCT | 96 |
| NOTCH1-chr9 R1 | TCCACCGTCTGAGGGAAAGCTCG | 97 |
| NOTCH1-GABBR2 F1 | GGTGAGGTTGACGCCGACTGCAT | 98 |
| NOTCH1-GABBR2 R1 | GACGATGCCAAGCCAGATGGTCATA | 99 |
| NOTCH2-SEC22B F1 | TGATGACTGCCCTAACCACAGGTGTC | 100 |
| NOTCH2-SEC22B R1 | TGGCTCCTGCTTCCAAGGTACATCTG | 101 |
| NOTCH cloning | | |
| NOTCH1-NICD-S | GAACGGTCCGACCATGCTGCTGTCCCGCAAGCG | 102 |
| NOTCH1-NICD-AS | GAACGGACCGAAGGCTTGGGAAAGGAAG | 103 |
| HCC1599-NOTCH1-NICD-F | TGAGACCTGCCTGAATGGCGGGAA | 104 |
| HCC1599-NOTCH1-NICD-R | GCCCACGAAGAACAGAAGCACAAAGG | 105 |
| HCC2218-NOTCH1-NICD-F | ACCCGAGCCGGATGTGCCAAGAT | 106 |
| HCC2218-NOTCH1-NICD-R | GCCGCCACGTACATGAAGTGCAG | 107 |
| HCC1187-NOTCH2-F | GAACGGTCCGACCATGGCAAAACGAAAGCGTAAGC | 108 |
| HCC1187-NOTCH2-R | CAACGGACCGGATGACCTTCATTTGTTCCTC | 109 |

TABLE 7

| Sample | Fusions | ER^{a} | PR^{a} | ERBB2^{a} | Source |
|---|---|---|---|---|---|
| Po [symbol] -BT1 | | − | − | − | U Michigan |
| Po [symbol] -BT2 | | + | + | + | U Michigan |
| Po [symbol] -BT3 | | + | + | − | U Michigan |
| Po [symbol] -BT4 | | + | − | − | U Michigan |
| Po [symbol] -BT5 | | + | + | − | U Michigan |
| Po [symbol] -BT6 | | − | − | − | U Michigan |
| Po [symbol] -BT7 | | − | − | + | U Michigan |
| Po [symbol] -BT8 | | + | − | − | U Michigan |
| Po [symbol] -BT9 | | + | − | − | U Michigan |
| Po [symbol] -BT10 | | + | + | + | U Michigan |
| Po [symbol] -BT11 | | + | + | + | U Michigan |
| Po [symbol] -BT12 | | + | − | + | U Michigan |
| Po [symbol] -BT13 | | + | + | − | U Michigan |
| Po [symbol] -BT14 | | − | − | − | U Michigan |
| Po [symbol] -BT15 | | + | + | − | U Michigan |
| Po [symbol] -BT16 | | − | − | + | U Michigan |
| Po [symbol] -BT17 | | + | + | − | U Michigan |
| Po [symbol] -BT18 (BrCa10038) | TADA2A-MAST1 | − | − | + | U Michigan |
| Po [symbol] -BT19 | | + | − | − | U Michigan |
| Po [symbol] -BT20 | | + | + | − | U Michigan |
| Po [symbol] -BT21 | | − | − | − | U Michigan |
| Po [symbol] -BT22 | | + | + | − | U Michigan |
| Po [symbol] -BT23 | | − | − | − | U Michigan |
| Po [symbol] -BT24 | | + | + | − | U Michigan |
| Po [symbol] -BT25 | | + | + | + | U Michigan |
| Po [symbol] -BT26 | | + | − | − | U Michigan |
| Po [symbol] -BT27 | | + | + | − | U Michigan |
| Po [symbol] -BT28 | | − | − | − | U Michigan |
| Po [symbol] -BT29 | | + | + | − | U Michigan |
| Po [symbol] -BT30 | | − | − | − | U Michigan |
| Po [symbol] -BT31 | | − | − | + | U Michigan |
| Po [symbol] -BT32 (BrCa10039) | GPBP1L1-MAST2 | + | − | − | U Michigan |
| Po [symbol] -BT33 | | + | − | − | U Michigan |
| Po [symbol] -BT34 | | + | + | − | U Michigan |
| Po [symbol] -BT35 | | + | − | − | Reis-Filho Lab^{a} |
| Po [symbol] -BT36 | | − | − | − | Reis-Filho Lab^{a} |
| Po [symbol] -BT37 | | + | + | − | Reis-Filho Lab^{a} |
| Po [symbol] -BT38 | | − | − | − | Reis-Filho Lab^{a} |
| Po [symbol] -BT39 | | + | − | − | Reis-Filho Lab^{a} |
| Po [symbol] -BT40 | | + | + | − | Reis-Filho Lab^{a} |
| Po [symbol] -BT41 | | − | − | + | Reis-Filho Lab^{a} |
| Po [symbol] -BT42 | | − | − | + | Reis-Filho Lab^{a} |
| Po [symbol] -BT43 | | + | + | − | Reis-Filho Lab^{a} |
| Po [symbol] -BT44 | | − | − | − | Reis-Filho Lab^{a} |
| Po [symbol] -BT45 | | − | − | + | Reis-Filho Lab^{a} |
| Po [symbol] -BT46 | | − | − | + | Reis-Filho Lab^{a} |
| Po [symbol] -BT47 | | + | + | − | Reis-Filho Lab^{a} |
| Po [symbol] -BT48 | | − | − | − | Reis-Filho Lab^{a} |
| Po [symbol] -BT49 | | − | − | − | Reis-Filho Lab^{a} |
| Po [symbol] -BT50 | | − | − | + | Reis-Filho Lab^{a} |
| Po [symbol] -BT51 | | + | − | − | Reis-Filho Lab^{a} |
| Po [symbol] -BT52 | | + | + | − | Reis-Filho Lab^{a} |
| Po [symbol] -BT53 | | + | + | − | Reis-Filho Lab^{a} |
| Po [symbol] -BT54 | | − | + | − | Reis-Filho Lab^{a} |
| Po [symbol] -BT55 | | − | − | + | Reis-Filho Lab^{a} |
| Po [symbol] -BT56 | | − | − | − | Reis-Filho Lab^{a} |
| Po [symbol] -BT57 | | + | + | − | Reis-Filho Lab^{a} |
| Po [symbol] -BT58 | | + | + | − | Reis-Filho Lab^{a} |
| Po [symbol] -BT59 | | − | − | − | Reis-Filho Lab^{a} |
| Po [symbol] -BT60 | | + | + | − | Reis-Filho Lab^{a} |
| Po [symbol] -BT61 | | − | − | − | Reis-Filho Lab^{a} |
| Po [symbol] -BT62 | | − | − | + | Reis-Filho Lab^{a} |
| Po [symbol] -BT63 | | − | − | − | Reis-Filho Lab^{a} |
| Po [symbol] -BT64 | | − | − | + | Reis-Filho Lab^{a} |
| Po [symbol] -BT65 | | + | − | − | Reis-Filho Lab^{a} |
| Po [symbol] -BT66 | | − | − | + | Reis-Filho Lab^{a} |
| Po [symbol] -BT67 | | − | − | + | Reis-Filho Lab^{a} |
| Po [symbol] -BT68 | | − | − | − | Reis-Filho Lab^{a} |
| Po [symbol] -BT69 | | + | + | − | Origene |
| Po [symbol] -BT70 | | − | − | − | Origene |
| Po [symbol] -BT71 | | − | − | − | Origene |
| Po [symbol] -BT72 | | + | − | + | Origene |
| Po [symbol] -BT73 | | + | − | − | Origene |
| Po [symbol] -BT74 | | + | − | − | Origene |

^{a}The ER/PR positivity and ERBB2 overexpression status are from clinical diagnosis.
^{b}Dr. Jorge Reis-Filho, The Breakthrough Breast Cancer Research Centre, Institute of Cancer Research, London, UK. [symbol]
indicates data missing or illegible when filed

TABLE 8

| Protein interrogated | Pathway | |
|---|---|---|
| (Antibody) | involved | Source |
| TRAIL | Apoptosis | Kinexus |
| c-IAP1 | Apoptosis | Kinexus |
| FAS | Apoptosis | Kinexus |
| Hsp40 | Chaperone | Kinexus |
| Tyk2 | JAK-STAT | Kinexus |
| STAT5B | JAK-STAT | Kinexus |
| STAT5A | JAK-STAT | Kinexus |
| STAT2 | JAK-STAT | Kinexus |
| TBK1 | NFkB | Kinexus |
| IKKb | NFkB | Kinexus |
| Abl | Non receptor tyrosine kinase | Kinexus |
| PI3K p85/p55 | PI3K | Kinexus |
| PI3K | PI3K | Kinexus |
| PRKCB | Protein kinase | Kinexus |
| Raf1 | RAS | Kinexus |
| TAK1 | TLR4 | Kinexus |
| phospho GSK | Wnt | Cell Signaling |
| phospho ERK1/2 | MAPK | Cell Signaling |
| total ERK1/2 | MAPK | Cell Signaling |
| phospho p38 | MAPK | Cell Signaling |
| phospho Akt | PI3K | Cell Signaling |
| total Akt | PI3K | Cell Signaling |
| PTEN | PI3K | Cell Signaling |

### Example 2

### Additional Breast Cancer Markers

Experiments were conducted to identify additional fusions in breast cancer. Experiments identified an FGFR fusion in breast cancer and functionally recurrent fusions of ETV6 in breast cancer.

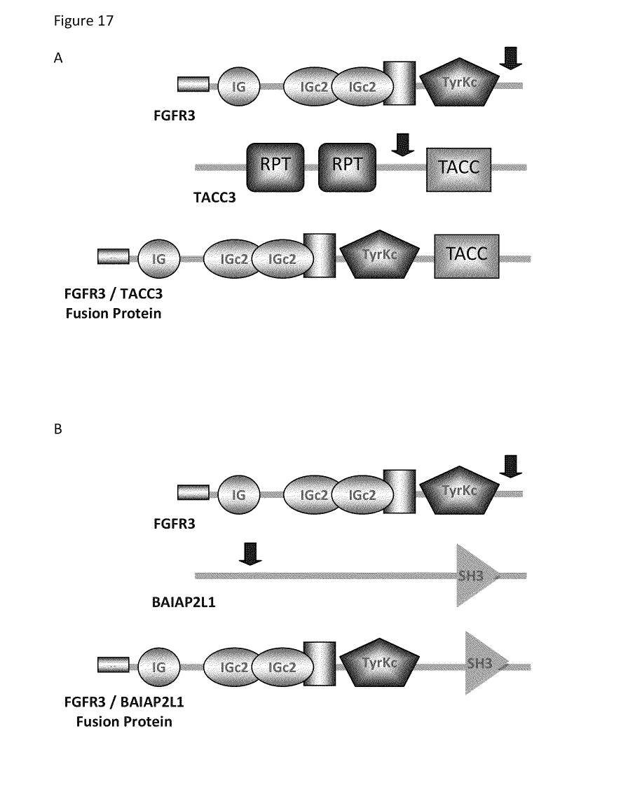

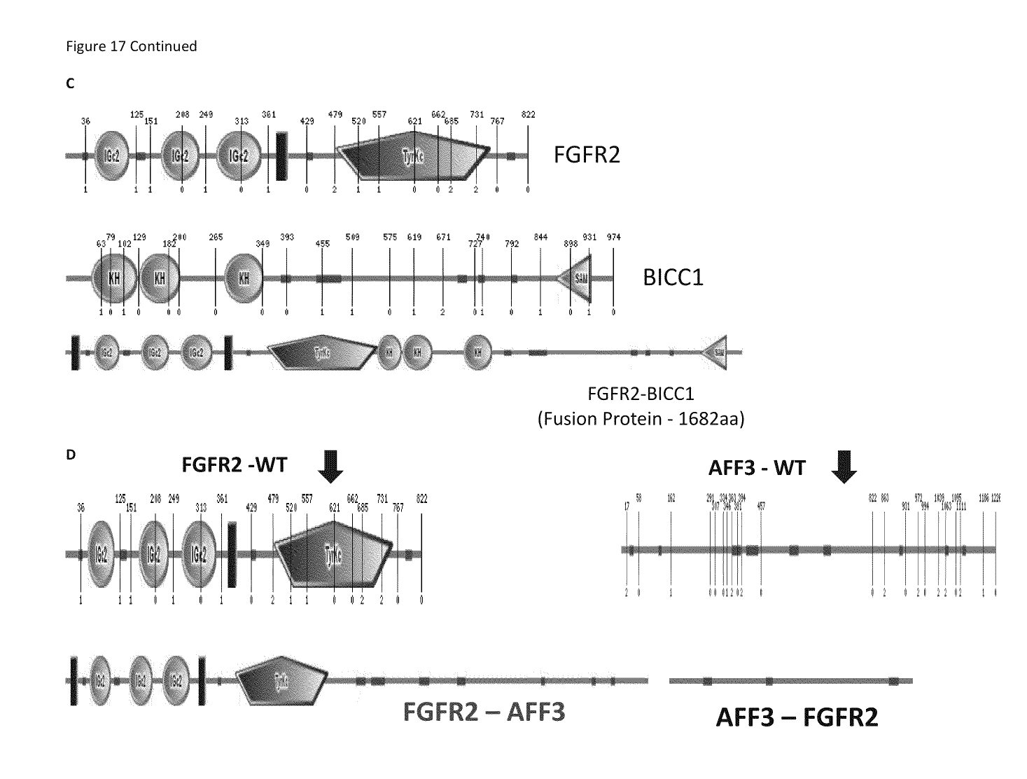

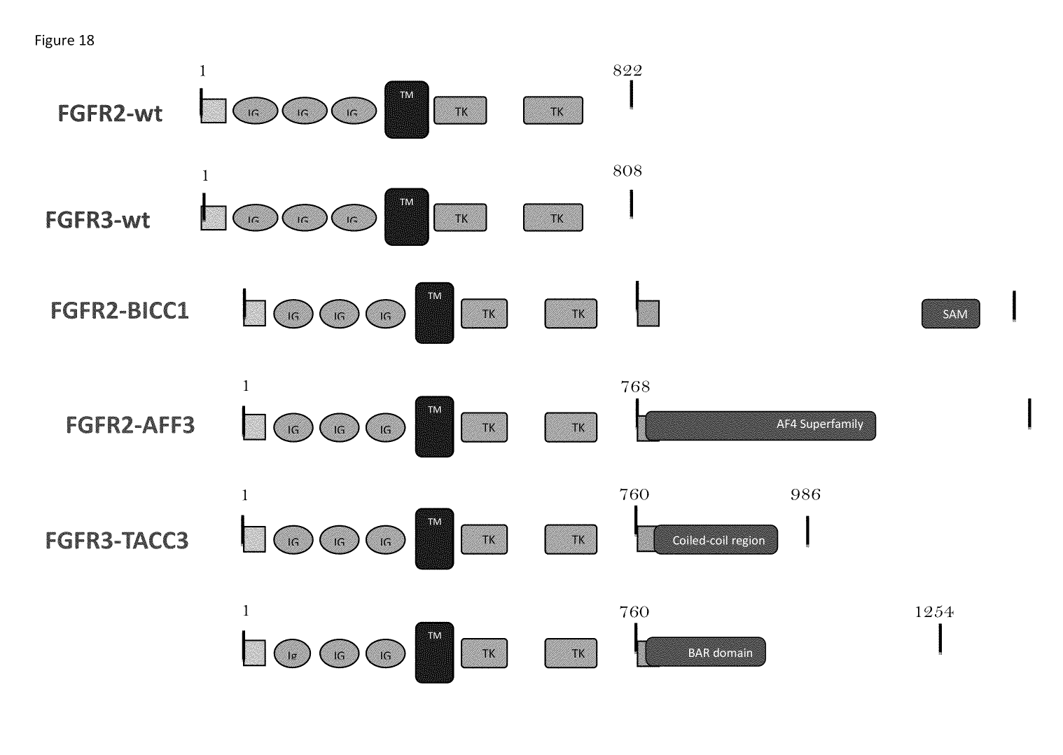

Table 9 shows FGFR3 fusions in a variety of cancers. FIGS. 17-18 show FGFR3 gene fusions.

| 5′ | 3′ | Sample | Tissue | Sample | Read |
|---|---|---|---|---|---|
| Gene | Gene | Name | Type | Type | # |
| FGFR3 | TACC3 | NC8 | Oral | Cell line | 87 |
| FGFR3 | TACC3 | NC9 | Oral | Cell line | 67 |
| FGFR3 | TACC3 | C010 | Lung | Tissue | 27 |
| FGFR3 | BAIAP2L1 | SW780 | Bladder | Cell line | 297 |
| FGFR2 | BICC1 | MO_1039 | Cholangio- | Tissue | 1041 |
| | | | carcinoma | | |
| FGFR2 | BICC1 | MO_1036 | Cholangio- | Tissue | 259 |
| | | | carcinoma | | |
| FGFR2 | AFF3 | MO_1051 | Breast | Tissue | 138 |

Fibroblast growth factors (FGFs) (FGF1-10 and 16-23) are mitogenic signaling molecules that have roles in angiogenesis, wound healing, cell migration, neural outgrowth and embryonic development. FGFs bind heparan sulfate glycosaminoglycans (HSGAGs), which facilitates dimerization (activation) of FGF receptors (FGFRs). FGFRs are transmembrane catalytic receptors that have intracellular tyrosine kinase activity.

Overexpression of fibroblast growth factor receptor 3 (FGFR3) has been shown to drive oncogenesis in a subset of patients with multiple myeloma. FGFR3 is an oncogenic driver of bladder cancer, indicating that FGFR3 has important roles in the oncogenesis of other epithelial cancers.

Table 10 shows ETV6 fusions in breast cancer

| 5′ Gene | 3′ Gene | Sample Name | Source | Reads # | Type |
|---|---|---|---|---|---|
| CIT | ETV6 | BrCa10038 | Origene | 57 | Intra |
| PEX5 | ETV6 | BrCa10038 | Origene | 149 | Intra |
| GTF2I | ETV7 | BrCa10058 | Origene | 6 | Intra |
| BCL2L14 | ETV6* | BrCa10025 | England | 6 | Intra |
| BCL2L14 | ETV6* | BrCa10071 | Origene | 102 | Intra |
| ETV6 | CD70 | BrCa10071 | Origene | 3 | Inter |
| ETV6 | SYN1 | BrCa10008 | Michigan | 16 | Inter |

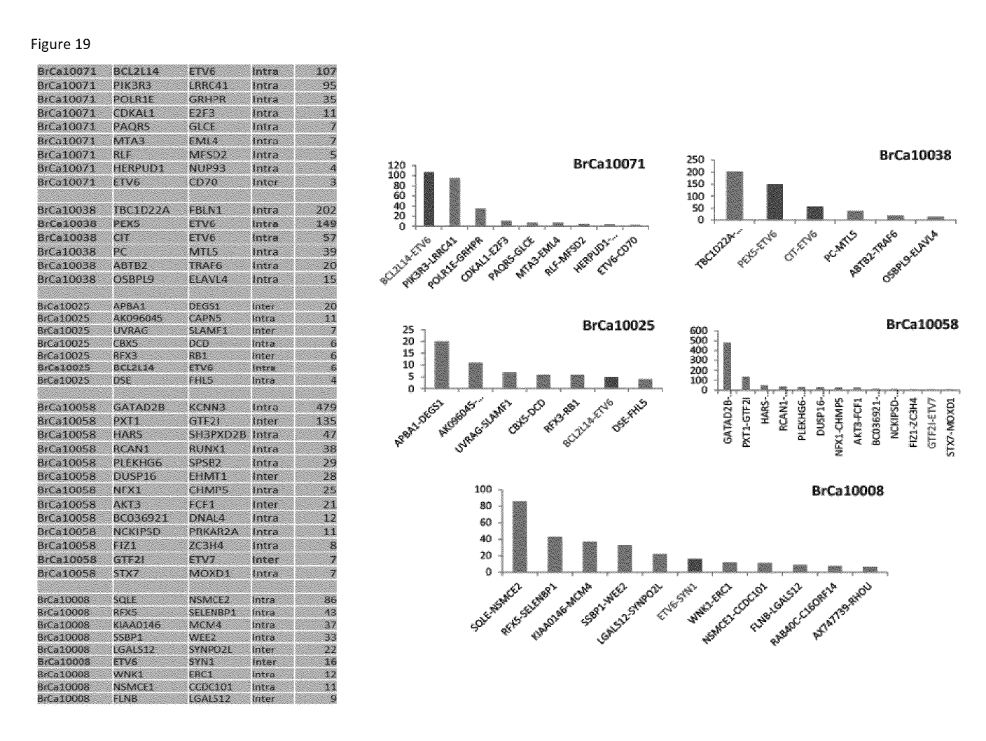

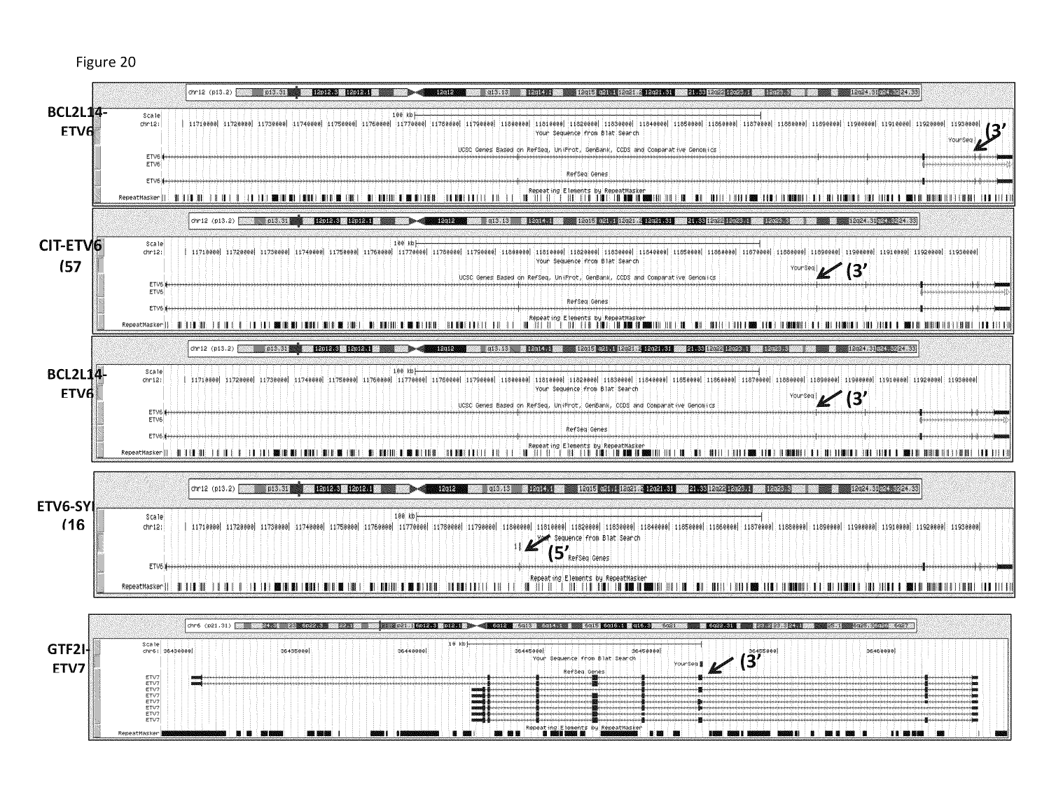

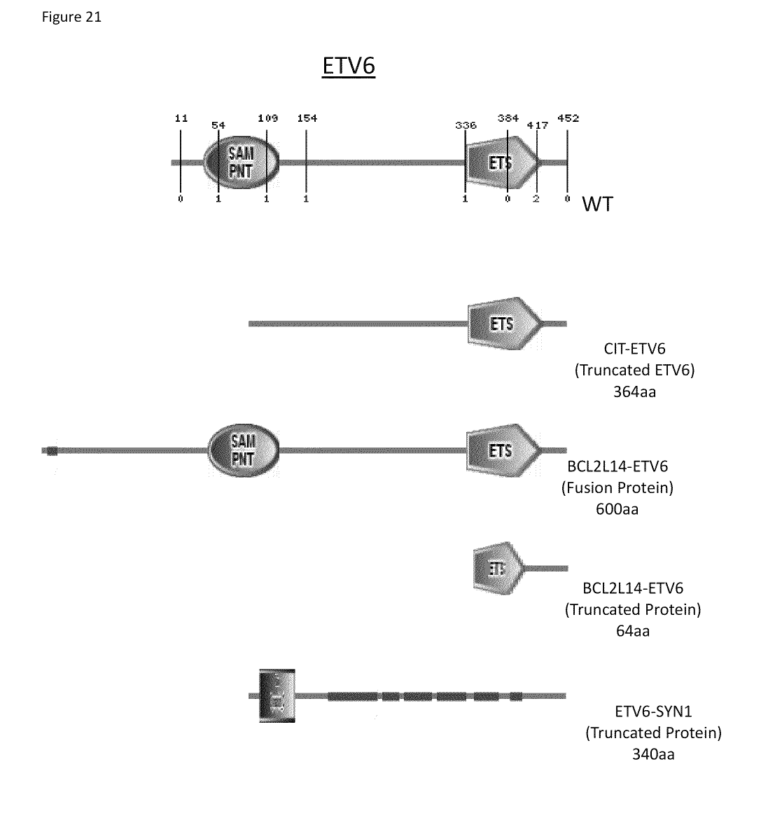

FIGS. 19-21 shows ETV6 fusions. ETV6/NTRK3 has been shown (Nature Genetics, Vol 18, February 1998; Cancer Research, Vol 58, November 1998; Blood Vol 93 February 1999; Cancer Cell, November 2002) to be a recurrent gene fusion in a variety of cancers.

Additional breast cancer gene fusions include, but are not limited to, CTNNA1-JMJD1B and RB1CC1-JAK1.

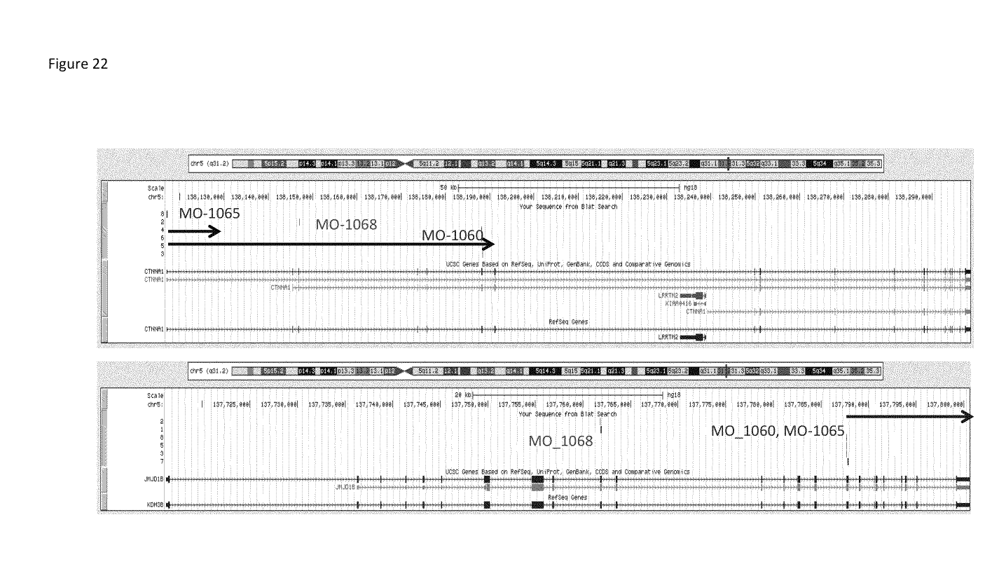

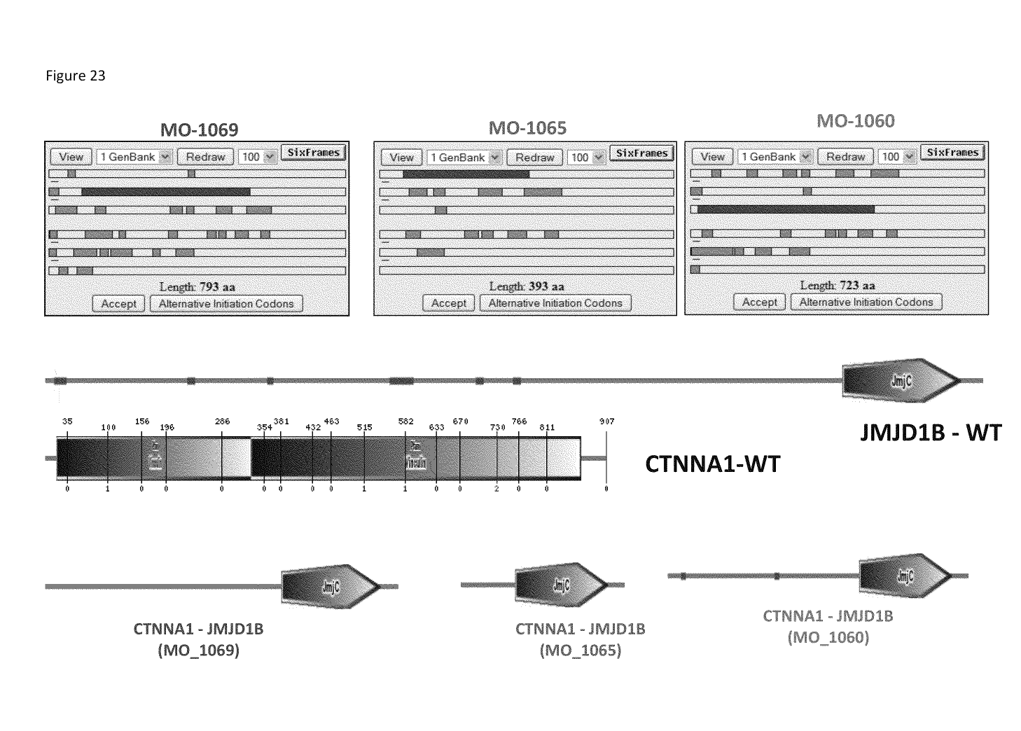

Table 11 and FIGS. 22-23 show CTNNA1-JMJD1B gene fusions in breast cancer.

| 5′ Gene | 3′ Gene | Sample Name | Tissue Type | Sample Type |
|---|---|---|---|---|
| CTNNA1 | JMJD1B | MO_1060 | Ovary | Tissue |
| CTNNA1 | JMJD1B | MO_1065 | Breast | Tissue |
| CTNNA1 | JMJD1B | MO_1069 | Breast | Tissue |

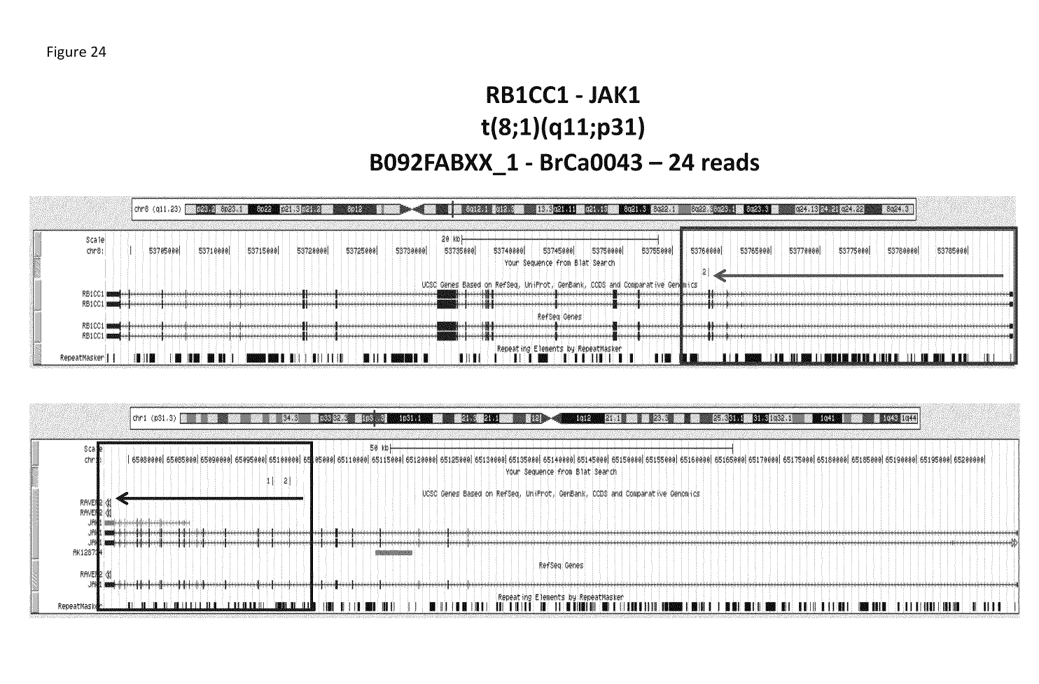

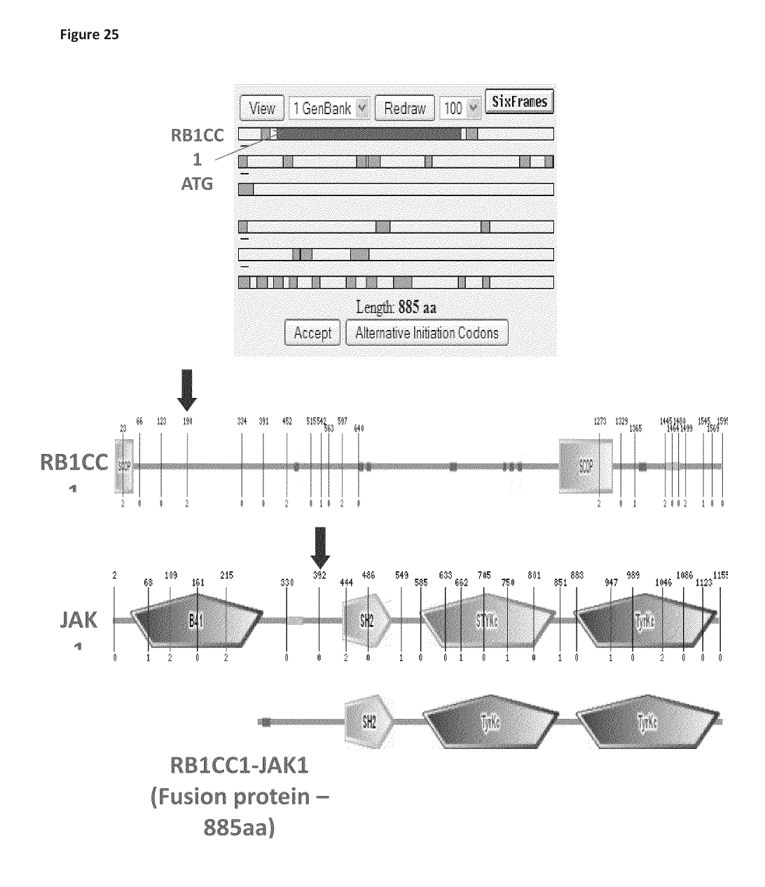

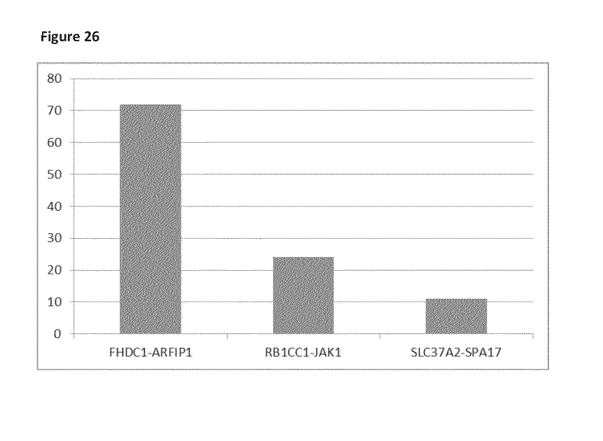

FIGS. 24-26 shows JAK kinase fusions in breast cancer.

Although a variety of embodiments have been described in connection with the present disclosure, it should be understood that the claimed invention should not be unduly limited to such specific embodiments. Indeed, various modifications and variations of the described compositions and methods of the invention will be apparent to those of ordinary skill in the art and are intended to be within the scope of the following claims.

We claim:

1. A kit for detecting gene fusions associated with cancer a subject, consisting essentially of at least a first gene fusion informative reagent for identification of a gene fusion selected from the group consisting of: ZNF700MAST1, NFIX-MAST1, ARID1A-MAST2, TADA2A-MAST1, GPBP1L1-MAST2, SEC16A-NOTCH1, SEC22B-NOTCH2, NOTCH1-GABRR2, NOTCH1-ch9:138722833, NOTCH1-SNHG7, NOTCH2-SEC22b, FGFR2-AFF3, CIT-ETV76, PEX5-ETV6, GTF2I-ETV7, BCL2L14-ETV6, ETV-CD70, ETV6-SYN1, CTNNA1-JMJD1B and RB1CC1-JAK1.

2. The kit of claim 1, wherein said reagent is a probe that specifically hybridizes to the fusion junction of said gene fusion.

3. The kit of claim 1, wherein said reagent is a pair of primers that amplify a fusion junction of said gene fusion.

4. The kit of claim 3, wherein said pair of primers comprise a first primer that hybridizes to a 5′ member of said gene fusion and second primer that hybridizes to a 3′ member of said gene fusion.

5. The kit of claim 1, wherein said reagent is an antibody that binds to the fusion junction of said gene fusion polypeptide.

6. The kit of claim 1, wherein the reagent is a sequencing primer that binds to said gene fusion and generates an extension product that spans the fusion junction of said gene fusion.

7. The kit of claim 1, wherein said regent comprises a pair of probes wherein said first probe hybridizes to a 5′ member of said gene fusion and said second probe hybridizes to a 3′ member of said gene fusion gene.

8. The kit of claim 1, wherein said reagent is labeled.

9. The kit of claim 1, wherein said cancer is breast cancer.

10. A method for identifying cancer in a patient comprising:
(a) contacting a biological sample form a subject with a nucleic acid or polypeptide detection assay comprising: at least a first gene fusion informative reagent for identification of a gene fusion selected from the group consisting of: ZNF700-MAST1, NFIX-MAST1-ARID1A-MAST2, TADA2A-MAST1, GPBP1L1-MAST2, SEC16A-NOTCH1, SEC22B-NOTCH2, NOTCH1-GABRR2, NOTCH1-ch9:138722833, NOTCH1-SNHG7, NOTCH2-SEC22b, FGFR2-AFF3, CIT-ETV6, PEX5-ETV6, GTF2I-ETV7, BCL2L14-ETV6, ETV-CD70, ETV6-SYN1, CTNNA1-JMJD1B and RB1CC1-JAK1; and
(b) identifying cancer in said subject when said gene fusion is present in said sample.

11. The method of claim 10, wherein the sample is selected from the group consisting of tissue, blood, plasma, serum, cells and tissues.

12. The method of claim 10, wherein the cancer is breast cancer.

13. The method of claim 10, further comprising the step of determining a treatment course of action based on the presence or absence of the gene fusion in the sample.

14. The method of claim 10, wherein the treatment course of action comprises administration of a gene fusion pathway inhibitor when said gene fusion is present in the sample.
